SEQUENCE LISTING (114 sequences)

SEQ ID NO: 1 - protein, 491 aa, Artificial Sequence
  gag.N16.1 ? optimized gag sequence derived from multiple HIV-1 isolate gag sequences
MGARASVLSGGKLDAWEKIRLRPGGKKKYRLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQALSPRTLNAWVKVIEEKAFSPEVIPMFTALSEGATPQDLNMMLNIVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPSHKARVLAEAMSQAQHTNIMMQRGNFRGQKRIKCFNCGKEGHLARNCRAPRKKGCWKCGKEGHQMKDCTERQANFLGKIWPSHKGRPGNFLQNRPEPTAPPAESFRFEETTPAPKQEPKDREPLTSLKSLFGSDPLSQ

SEQ ID NO: 2 - protein, 498 aa, Artificial Sequence
  gag.N16.2 ? optimized gag sequence derived from multiple HIV-1 isolate gag sequences
MGARASILRGGKLDKWEKIRLRPGGKKHYMLKHLVWASRELERFALNPSLLETSEGCKQIMKQLQPALQTGTEELRSLYNTVATLYCVHQRIDVKDTKEALDKIEEEQNKSKKKAQQAAADTGNSSQVSQNYPIVQNAQGQMVHQPLSPRTLNAWVKVVEEKGFNPEVIPMFTALSEGATPQDLNTMLNTVGGHQAAMQMLKDTINEEAAEWDRVHPVHAGPIPPGQMREPRGSDIAGTTSTPQEQIGWMTSNPPIPVGEIYKRWIIMGLNKIVRMYSPVSILDIRQGPKEPFRDYVDRFFKTLRAEQATQEVKNWMTETLLVQNANPDCKSILKALGTGATLEEMMTACQGVGGPGHKARVLAEAMSQANSNILMQRSNFKGSKRIVKCFNCGKEGHIARNCRAPRKKGCWKCGREGHQMKDCTERQANFLGKIWPSSKGRPGNFPQSRPEPTAPPAESFRFGEETTTPSQKQEPIDKELYPLASLKSLFGNDPSSQ

SEQ ID NO: 3 - protein, 206 aa, Artificial Sequence
  nef.N16.1 ? optimized nef sequence derived from multiple HIV-1 isolate nef sequences
MGGKWSKSSIVGWPAVRERIRRTEPAAEGVGAASQDLDKYGALTSSNTAANNADCAWLEAQEEEEVGFPVRPQVPLRPMTYKAAFDLSFFLKEKGGLEGLIYSKKRQEILDLWVYHTQGFFPDWQNYTPGPGVRYPLTFGWCFKLVPVDPREVEEANEGENNCLLHPMSQHGMDDPEKEVLVWKFDSRLAFHHMARELHPEYYKDC

SEQ ID NO: 4 - protein, 206 aa, Artificial Sequence
  nef.N16.2 ? optimized nef sequence derived from multiple HIV-1 isolate nef sequences
MGGKWSKSSIVGWPAIRERIRRTDPAAEGVGAASQDLDKHGALTSSNTATNNAACAWLEAQEEEEVGFPVKPQVPLRPMTYKGALDLSHFLKEKGGLEGLIYSQKRQDILDLWVYNTQGYFPDWQNYTPGPGIRYPLTFGWCFKLVPVDPDEVEKATEGENNSLLHPICQHGMDDEEKEVLMWKFDSSLARRHMARELHPEYYKDC

SEQ ID NO: 5 - protein, 38 aa, Artificial Sequence
  amino acids in gag from HIV-1 isolate 00BW1921.13 that differ from gag.N16.1
IRTRMITDQYKGQEASITTILNVSSEANNKSKPRIVIS

SEQ ID NO: 6 - protein, 39 aa, Artificial Sequence
  amino acids in gag from HIV-1 isolate SM145 that differ from gag.N16.1
IRRHMIMIITTQDSANNILKSIVIPTAPPAESFRVRPEL

SEQ ID NO: 7 - protein, 53 aa, Artificial Sequence
  amino acids in gag from HIV-1 clone M428gag that differ from gag.N16.1
ISTQLEGQRIKKKTDTNSSKAVVSIPGAEGESKTRSPLDGMRIASPQQPQINL

SEQ ID NO: 8 - protein, 29 aa, Artificial Sequence
  amino acids in gag from HIV-1 strain 98IN012 that differ from gag.N16.1
IRKHMAADEKVTTNRSATGSTSKSTVIAL

SEQ ID NO: 9 - protein, 63 aa, Artificial Sequence
  amino acids in gag from HIV-1 unidentified gag isolate #1 that differ from gag.N16.1
ISQGSSNKYQRDKIKADTRNSNQAPTMVTLGNECVRISEHESKTVHNKNNGNTPSGGIEKLYA

SEQ ID NO: 10 - protein, 56 aa, Artificial Sequence
  amino acids in gag from HIV-1 isolate NY5 that differ from gag.N16.2
VSEQRIVGRLRSSRFVALAIASSELALHLTYSTPAVTNPATMIGRNQKTKKHLERS

SEQ ID NO: 11 - protein, 56 aa, Artificial Sequence
  amino acids in gag from HIV-1 unidentified gag isolate #2 that differ from gag.N16.2
TKIFEKERSRTQQEADKGKLAIASQAIGLADLKDTRPVNPMSKHLEIGPKRPPTSL

SEQ ID NO: 12 - protein, 75 aa, Artificial Sequence
  amino acids in gag from HIV-1 unidentified gag isolate #3 that differ from gag.N16.2
VTAKRIGAQLLESTKSKFIWREVKQQTSKSTMIELLDLYSKQQVMDQKRIRLKILNNWGMEIPLLRQKDPPPSVL

SEQ ID NO: 13 - protein, 60 aa, Artificial Sequence
  amino acids in gag from HIV-1 clone TV008G65 that differ from gag.N16.2
VETRIGIQKFKKERSKTQQEAADKKLAIAGALTALARVDLKGDTRPGKEHLEAPSKRPTR

SEQ ID NO: 14 - protein, 81 aa, Artificial Sequence
  amino acids in gag from HIV-1 isolate 00BW3842.8 that differ from gag.N16.2
VETRKIGMIQFIKGKQQQTQQKTQQTEAAAGKLAIQVAINLTVLDTRPSSTMGPTILKRGLNTEPTAPEAPKRGPREPISL

SEQ ID NO: 15 - protein, 58 aa, Artificial Sequence
  amino acids in gag from HIV-1 unidentified gag isolate #4 that differ from gag.N16.2
GAIHKDERSKTQQEAADKKLAIIASLVALADLKSDNRPSSNPMKPRKLKETPKRPTSL

SEQ ID NO: 16 - protein, 66 aa, Artificial Sequence
  amino acids in gag from HIV-1 patient R2 that differ from gag.N16.2
VSRKKIVGRLGSSKFIGEAANPLAIASSELANLANLTKYSDTPAVTNNPVMGRNQRKTLKEHLEAP

SEQ ID NO: 17 - protein, 59 aa, Artificial Sequence
  amino acids in gag from HIV-1 isolate MBC200 that differ from gag.N16.2
VSERQRIVGRLGSKSIKKEEAELAIASSELALNLTYSTPVTNSATMGRNQRKTKRKYLR

SEQ ID NO: 18 - protein, 58 aa, Artificial Sequence
  amino acids in gag from HIV-1 clone M21_ps1 that differ from gag.N16.2
VSAKRTQQSEIKQTISIASSMILDLKGIARSVQQTMGQKRILKNIGMEISPPQAPPVL

SEQ ID NO: 19 - protein, 66 aa, Artificial Sequence
  amino acids in gag from HIV-1 unidentified gag isolate #5 that differ from gag.N16.2
TCIGIQKFAKERCQQTAKKAADETSIIASIALRAGADLKDDTRPSSTSTMNKHENAPKARERPISL

SEQ ID NO: 20 - protein, 74 aa, Artificial Sequence
  amino acids in gag from HIV-1 unidentified gag isolate #6 that differ from gag.N16.2
VSARKRIQIGSSNKIQTRNPTVMIASMITVALLNLCVISHSVHNTMKGRQKRILKNNNTGEIAKEPKEKELYPS

SEQ ID NO: 21 - protein, 57 aa, Artificial Sequence
  amino acids in gag from HIV-1 isolate 1018-10 that differ from gag.N16.2
SQRQKIGVGRISSFCEALAITASSELALYSDTPAVTNSTTVMGNQRKTILKEHLETY

SEQ ID NO: 22 - protein, 57 aa, Artificial Sequence; identical to SEQ ID NO: 21
  amino acids in gag from HIV-1 isolate 90CF402 that differ from gag.N16.2

SEQ ID NO: 23 - protein, 68 aa, Artificial Sequence
  amino acids in gag from HIV-1 unidentified gag isolate #7 that differ from gag.N16.2
RGPISNERCQQTKAQQEAAAEKLSIILAVASNLTVDLKDDTRPSSNPHVMKRKEHLMEAAPSTRPTLL

SEQ ID NO: 24 - protein, 68 aa, Artificial Sequence; identical to SEQ ID NO: 23
  amino acids in nef from HIV-1 clone SP2-12 that differ from nef.N16.1

SEQ ID NO: 25 - protein, 68 aa, Artificial Sequence; identical to SEQ ID NO: 23
  amino acids in nef from HIV-1 isolate 96BW11 clone B01 that differ from nef.N16.1

SEQ ID NO: 26 - protein, 27 aa, Artificial Sequence
  amino acids in nef from HIV-1 isolate patient 50219 clone 2 that differ from nef.N16.1
MFIKAEPAADREHTAVHHQDYIYEEQK

SEQ ID NO: 27 - protein, 14 aa, Artificial Sequence
  amino acids in nef from HIV-1 strain 96ZM651 that differ from nef.N16.1
STTAVGQDHRKHHK

SEQ ID NO: 28 - protein, 26 aa, Artificial Sequence
  amino acids in nef from HIV-1 unidentified nef isolate #1 that differ from nef.N16.1
NCSLTPTANNPAQQGDNYIARQSRRF

SEQ ID NO: 29 - protein, 21 aa, Artificial Sequence
  amino acids in nef from HIV-1 TV003 that differ from nef.N16.1
KQQEVDHQDKDGIVHMHRTIF

SEQ ID NO: 30 - protein, 33 aa, Artificial Sequence
  amino acids in nef from HIV-1 clone SP2-12 that differ from nef.N16.2
TKITVMAEPAAEVREIDREEKENCSLEPERRFH

SEQ ID NO: 31 - protein, 35 aa, Artificial Sequence
  amino acids in nef from HIV-1 isolate 00KE_KSM4024 that differ from nef.N16.2
SRTMEEVMCASPVVVNHPSVKRAFFDKFQHKRLFN

SEQ ID NO: 32 - protein, 34 aa, Artificial Sequence
  amino acids in nef from HIV-1 unidentified nef isolate #2 that differ from nef.N16.2
EPATVYVINPSTQSDAGRFFDHHTYAIETCSIDF

SEQ ID NO: 33 - protein, 41 aa, Artificial Sequence
  amino acids in nef from HIV-1 isolate 00BW2127.214 that differ from nef.N16.2
IEDQSEGRAFFKEHFVKEKENDCMSRQLSLIDTCGVLQRLL

SEQ ID NO: 34 - protein, 35 aa, Artificial Sequence
  amino acids in nef from HIV-1 isolate AD8 that differ from nef.N16.2
RMATVMTAEDRERAVHEHVEEQIENKCMSTRQRFH

SEQ ID NO: 35 - protein, 36 aa, Artificial Sequence
  amino acids in nef from HIV-1 unidentified nef isolate #3 that differ from nef.N16.2
EMAKRTPHDQERRAFPGFDHKHVFYQENDCMSIEAD

SEQ ID NO: 36 - protein, 27 aa, Artificial Sequence
  amino acids in nef from HIV-1 isolate 02UZ0659 that differ from nef.N16.2
QVAPAPARPVVADIDKEHFYEERLTRF

SEQ ID NO: 37 - protein, 29 aa, Artificial Sequence
  amino acids in nef from HIV-1 patient MP169 that differ from nef.N16.2
ATDQETAVWKEIHCVFGENCMSEDRKHHF

SEQ ID NO: 38 - protein, 32 aa, Artificial Sequence
  amino acids in nef from HIV-1 clone MB10 that differ from nef.N16.2
RVVMTIEDVREIRVIHKTCEENSLELVRRFHV

SEQ ID NO: 39 - DNA, 1473 bp, Artificial Sequence
  gag.N16.1 ? optimized gag sequence derived from multiple HIV-1 isolate gag sequences
atgggcgctcgcgcctctgtgctgtctggcggcaagctggatgcctgggagaagattcggctgcggcctggcgggaagaagaagtaccggctgaagcatctggtctgggccagccgcgagctggagcggtttgctctgaaccctggcctgctggagacctctgaaggctgtaagcagatcatcaagcagctgcagcctgccctgcaaacaggcacagaagagctgagatccctgttcaacacagtggccaccctgtactgtgtgcatgagaagattgaagtgcgcgacaccaaagaggccctggacaagattgaagaagaacagaacaagtcccagcagaagacccagcaggccaaagctgctgatggcaaagtgtcccagaactaccccatcgtgcagaacctgcagggccagatggtgcatcaggccctgtccccacggaccctgaatgcctgggtcaaagtgattgaagagaaagccttctcccctgaagtgatccccatgttcaccgccctgtctgaaggcgccacaccccaggacctgaacatgatgctgaacattgtgggcggccaccaggctgccatgcagatgctcaaagacaccatcaatgaagaggctgctgagtgggaccggctgcatcctgtccatgctggccctgtggcccctggccagatgcgcgagcctcgcggctctgacattgctggcacaacctccaccctgcaggagcagattgcctggatgaccagcaacccccccatccctgtgggcgacatctacaagagatggatcatcctgggcctgaacaagattgtgagaatgtactcccctgtgtccattctggacatcaagcagggccccaaagagccatttcgcgactatgtggaccggttctttaagaccctgcgcgctgagcaggctacccaggatgtgaagaactggatgacagacaccctgctggtccagaatgccaaccctgactgcaagaccatcctgcgcgccctgggccctggcgccaccctcgaagagatgatgacagcttgccagggcgtgggcggcccctcccacaaagccagagtgctggctgaagccatgagccaggcccagcacaccaacatcatgatgcagcgcggcaacttccgcggccagaagcggatcaagtgcttcaactgtggcaaggaaggccatctggcccggaactgccgcgcccccagaaagaaaggctgctggaagtgtggcaaagaaggccatcaaatgaaagactgcacagagcggcaagccaacttcctgggcaagatttggccctcccacaaaggccggcctggcaacttcctgcagaaccggcctgagcctacagccccccctgctgagagcttccggtttgaagagaccacccctgcccccaagcaggagcccaaagaccgcgagcccctgacctccctgaaatccctgtttggctctgaccccctgtcccag

SEQ ID NO: 40 - DNA, 1494 bp, Artificial Sequence
  gag.N16.2 ? optimized gag sequence derived from multiple HIV-1 isolate gag sequences
atgggcgcccgcgcctccatcctgcgcggcggcaaactggacaagtgggagaagatccggctgaggcctggcggcaagaagcactacatgctgaagcatctcgtctgggcctcccgcgagctcgagcggtttgccctgaacccctccctgctggagacatctgaaggctgcaagcagatcatgaagcagctgcaacctgccctgcagacaggcacagaggagctgcggtccctgtacaacacagtggcgaccctgtactgcgtgcatcagcggattgatgtgaaagacaccaaagaagccctggacaaaattgaagaagagcagaacaagtccaagaagaaagcccagcaggctgctgctgacacaggcaactcctcccaggtgtcccagaattaccccattgtgcagaatgcccagggccaaatggtgcatcagcccctgtccccccggaccctgaacgcctgggtgaaagtggtggaagagaaaggcttcaaccctgaagtgattcccatgttcacagccctgtctgagggcgccaccccccaggatctgaacaccatgctgaacacagtgggcggccatcaggctgctatgcagatgctgaaagacaccattaatgaagaagctgctgaatgggaccgcgtgcatcctgtgcatgctggccccatcccccctggccaaatgcgcgagccccgcggctctgatattgctggcaccacctccaccccccaggagcaaattggctggatgacctccaacccccccattcctgtgggcgaaatctacaagcggtggatcatcatgggcctgaataagattgtgcggatgtactcccccgtgtccatcctggacatccggcagggccctaaagagccattccgcgactatgtcgaccggttcttcaagaccctgagagctgagcaggccacccaggaagtgaagaattggatgacagagaccctgctggtgcagaatgccaatcctgactgcaagtccatcctgaaagccctgggcacaggcgccaccctggaagaaatgatgacagcctgccagggggtgggcggccctggccacaaagcccgcgtgctggctgaggccatgtcccaggccaactccaacatcctgatgcagcggtccaacttcaaaggctccaagcggattgtgaagtgctttaactgtggcaaagaaggccacattgcccggaactgtcgcgccccccggaagaaaggctgttggaagtgtggccgcgaaggccatcagatgaaagactgtacagagcggcaggccaacttcctcggcaaaatctggccctcctccaaaggccggcccggcaacttcccccagtcccggcctgagcccacagccccccccgctgagtccttccggtttggcgaagagaccaccaccccctcccagaagcaggaacccattgacaaagagctgtaccccctggcctccctgaagtccctgtttggcaatgacccctcctcccag

SEQ ID NO: 41 - DNA, 618 bp, Artificial Sequence
  nef.N16.1 ? optimized nef sequence derived from multiple HIV-1 isolate nef sequences
atggctggcaaatggtccaagtcctccattgtggggtggcctgctgtgcgcgagcggatccggcggacagagcctgctgctgagggcgtgggcgctgcctcccaggatctggacaagtatggcgccctgacctccagcaacacagctgccaacaatgctgactgtgcctggctggaagcccaggaggaagaagaagtgggctttcctgtgcggccccaggtgcccctgcggccaatgacctacaaagctgcctttgacctgtccttcttcctgaaagaaaaaggcggcctggaaggcctgatttactccaagaagcggcaggagatcctggacctgtgggtctaccacacccagggcttcttccctgattggcagaactacacccctgggcctggcgtgcggtaccccctgacctttgggtggtgcttcaaactggtgcctgtggacccccgcgaagtggaagaagccaatgaaggcgaaaacaactgcctgctgcatcccatgtcccagcatgggatggatgaccctgagaaagaagtgctggtctggaagttcgactcccggctggccttccaccacatggcccgggagctgcatcctgagtactataaagactgc

SEQ ID NO: 42 - DNA, 618 bp, Artificial Sequence
  nef.N16.2 ? optimized nef sequence derived from multiple HIV-1 isolate nef sequences
atggctggcaagtggtccaagtccagcattgtgggctggcctgccatccgcgagcggattcggcggacagaccctgctgctgaaggcgtgggcgccgcctcccaggacctggacaagcatggcgccctcacctcctccaacacagccaccaacaatgctgcctgtgcctggctcgaagcccaggaagaagaagaggtgggcttccctgtgaagccccaggtgcctctgcggcccatgacctataaaggcgccctggacctgtcccacttcctgaaagagaaaggcggcctcgaaggcctgatctactcccagaagcggcaggacatcctggacctctgggtctacaacacccagggctacttccctgactggcagaactatacccctggccctggcatccggtaccccctcacctttggctggtgcttcaagctggtgcccgtggaccctgatgaagtggagaaagccacagaaggcgagaacaactccctgctgcaccccatctgccagcatggcatggatgatgaagagaaagaggtgctgatgtggaagtttgactcctccctggcccggcggcacatggcccgcgagctgcatcccgagtactacaaagactgc

SEQ ID NO: 43 - DNA, 4203 bp, Artificial Sequence
  HIV-1 GAG(G1)GAG(G2)NEF(G1)NEF(G2) expression cassette
atgggcgctcgcgcctctgtgctgtctggcggcaagctggatgcctgggagaagattcggctgcggcctggcgggaagaagaagtaccggctgaagcatctggtctgggccagccgcgagctggagcggtttgctctgaaccctggcctgctggagacctctgaaggctgtaagcagatcatcaagcagctgcagcctgccctgcaaacaggcacagaagagctgagatccctgttcaacacagtggccaccctgtactgtgtgcatgagaagattgaagtgcgcgacaccaaagaggccctggacaagattgaagaagaacagaacaagtcccagcagaagacccagcaggccaaagctgctgatggcaaagtgtcccagaactaccccatcgtgcagaacctgcagggccagatggtgcatcaggccctgtccccacggaccctgaatgcctgggtcaaagtgattgaagagaaagccttctcccctgaagtgatccccatgttcaccgccctgtctgaaggcgccacaccccaggacctgaacatgatgctgaacattgtgggcggccaccaggctgccatgcagatgctcaaagacaccatcaatgaagaggctgctgagtgggaccggctgcatcctgtccatgctggccctgtggcccctggccagatgcgcgagcctcgcggctctgacattgctggcacaacctccaccctgcaggagcagattgcctggatgaccagcaacccccccatccctgtgggcgacatctacaagagatggatcatcctgggcctgaacaagattgtgagaatgtactcccctgtgtccattctggacatcaagcagggccccaaagagccatttcgcgactatgtggaccggttctttaagaccctgcgcgctgagcaggctacccaggatgtgaagaactggatgacagacaccctgctggtccagaatgccaaccctgactgcaagaccatcctgcgcgccctgggccctggcgccaccctcgaagagatgatgacagcttgccagggcgtgggcggcccctcccacaaagccagagtgctggctgaagccatgagccaggcccagcacaccaacatcatgatgcagcgcggcaacttccgcggccagaagcggatcaagtgcttcaactgtggcaaggaaggccatctggcccggaactgccgcgcccccagaaagaaaggctgctggaagtgtggcaaagaaggccatcaaatgaaagactgcacagagcggcaagccaacttcctgggcaagatttggccctcccacaaaggccggcctggcaacttcctgcagaaccggcctgagcctacagccccccctgctgagagcttccggtttgaagagaccacccctgcccccaagcaggagcccaaagaccgcgagcccctgacctccctgaaatccctgtttggctctgaccccctgtcccagatgggcgcccgcgcctccatcctgcgcggcggcaaactggacaagtgggagaagatccggctgaggcctggcggcaagaagcactacatgctgaagcatctcgtctgggcctcccgcgagctcgagcggtttgccctgaacccctccctgctggagacatctgaaggctgcaagcagatcatgaagcagctgcaacctgccctgcagacaggcacagaggagctgcggtccctgtacaacacagtggcgaccctgtactgcgtgcatcagcggattgatgtgaaagacaccaaagaagccctggacaaaattgaagaagagcagaacaagtccaagaagaaagcccagcaggctgctgctgacacaggcaactcctcccaggtgtcccagaattaccccattgtgcagaatgcccagggccaaatggtgcatcagcccctgtccccccggaccctgaacgcctgggtgaaagtggtggaagagaaaggcttcaaccctgaagtgattcccatgttcacagccctgtctgagggcgccaccccccaggatctgaacaccatgctgaacacagtgggcggccatcaggctgctatgcagatgctgaaagacaccattaatgaagaagctgctgaatgggaccgcgtgcatcctgtgcatgctggccccatcccccctggccaaatgcgcgagccccgcggctctgatattgctggcaccacctccaccccccaggagcaaattggctggatgacctccaacccccccattcctgtgggcgaaatctacaagcggtggatcatcatgggcctgaataagattgtgcggatgtactcccccgtgtccatcctggacatccggcagggccctaaagagccattccgcgactatgtcgaccggttcttcaagaccctgagagctgagcaggccacccaggaagtgaagaattggatgacagagaccctgctggtgcagaatgccaatcctgactgcaagtccatcctgaaagccctgggcacaggcgccaccctggaagaaatgatgacagcctgccagggggtgggcggccctggccacaaagcccgcgtgctggctgaggccatgtcccaggccaactccaacatcctgatgcagcggtccaacttcaaaggctccaagcggattgtgaagtgctttaactgtggcaaagaaggccacattgcccggaactgtcgcgccccccggaagaaaggctgttggaagtgtggccgcgaaggccatcagatgaaagactgtacagagcggcaggccaacttcctcggcaaaatctggccctcctccaaaggccggcccggcaacttcccccagtcccggcctgagcccacagccccccccgctgagtccttccggtttggcgaagagaccaccaccccctcccagaagcaggaacccattgacaaagagctgtaccccctggcctccctgaagtccctgtttggcaatgacccctcctcccagatggctggcaaatggtccaagtcctccattgtggggtggcctgctgtgcgcgagcggatccggcggacagagcctgctgctgagggcgtgggcgctgcctcccaggatctggacaagtatggcgccctgacctccagcaacacagctgccaacaatgctgactgtgcctggctggaagcccaggaggaagaagaagtgggctttcctgtgcggccccaggtgcccctgcggccaatgacctacaaagctgcctttgacctgtccttcttcctgaaagaaaaaggcggcctggaaggcctgatttactccaagaagcggcaggagatcctggacctgtgggtctaccacacccagggcttcttccctgattggcagaactacacccctgggcctggcgtgcggtaccccctgacctttgggtggtgcttcaaactggtgcctgtggacccccgcgaagtggaagaagccaatgaaggcgaaaacaactgcctgctgcatcccatgtcccagcatgggatggatgaccctgagaaagaagtgctggtctggaagttcgactcccggctggccttccaccacatggcccgggagctgcatcctgagtactataaagactgcatggctggcaagtggtccaagtccagcattgtgggctggcctgccatccgcgagcggattcggcggacagaccctgctgctgaaggcgtgggcgccgcctcccaggacctggacaagcatggcgccctcacctcctccaacacagccaccaacaatgctgcctgtgcctggctcgaagcccaggaagaagaagaggtgggcttccctgtgaagccccaggtgcctctgcggcccatgacctataaaggcgccctggacctgtcccacttcctgaaagagaaaggcggcctcgaaggcctgatctactcccagaagcggcaggacatcctggacctctgggtctacaacacccagggctacttccctgactggcagaactatacccctggccctggcatccggtaccccctcacctttggctggtgcttcaagctggtgcccgtggaccctgatgaagtggagaaagccacagaaggcgagaacaactccctgctgcaccccatctgccagcatggcatggatgatgaagagaaagaggtgctgatgtggaagtttgactcctccctggcccggcggcacatggcccgcgagctgcatcccgagtactacaaagactgc

SEQ ID NO: 44 - DNA, 4203 bp, Artificial Sequence
  HIV-1 GAG(G1)NEF(G1)GAG(G2)NEF(G2) expression cassette
atgggcgctcgcgcctctgtgctgtctggcggcaagctggatgcctgggagaagattcggctgcggcctggcgggaagaagaagtaccggctgaagcatctggtctgggccagccgcgagctggagcggtttgctctgaaccctggcctgctggagacctctgaaggctgtaagcagatcatcaagcagctgcagcctgccctgcaaacaggcacagaagagctgagatccctgttcaacacagtggccaccctgtactgtgtgcatgagaagattgaagtgcgcgacaccaaagaggccctggacaagattgaagaagaacagaacaagtcccagcagaagacccagcaggccaaagctgctgatggcaaagtgtcccagaactaccccatcgtgcagaacctgcagggccagatggtgcatcaggccctgtccccacggaccctgaatgcctgggtcaaagtgattgaagagaaagccttctcccctgaagtgatccccatgttcaccgccctgtctgaaggcgccacaccccaggacctgaacatgatgctgaacattgtgggcggccaccaggctgccatgcagatgctcaaagacaccatcaatgaagaggctgctgagtgggaccggctgcatcctgtccatgctggccctgtggcccctggccagatgcgcgagcctcgcggctctgacattgctggcacaacctccaccctgcaggagcagattgcctggatgaccagcaacccccccatccctgtgggcgacatctacaagagatggatcatcctgggcctgaacaagattgtgagaatgtactcccctgtgtccattctggacatcaagcagggccccaaagagccatttcgcgactatgtggaccggttctttaagaccctgcgcgctgagcaggctacccaggatgtgaagaactggatgacagacaccctgctggtccagaatgccaaccctgactgcaagaccatcctgcgcgccctgggccctggcgccaccctcgaagagatgatgacagcttgccagggcgtgggcggcccctcccacaaagccagagtgctggctgaagccatgagccaggcccagcacaccaacatcatgatgcagcgcggcaacttccgcggccagaagcggatcaagtgcttcaactgtggcaaggaaggccatctggcccggaactgccgcgcccccagaaagaaaggctgctggaagtgtggcaaagaaggccatcaaatgaaagactgcacagagcggcaagccaacttcctgggcaagatttggccctcccacaaaggccggcctggcaacttcctgcagaaccggcctgagcctacagccccccctgctgagagcttccggtttgaagagaccacccctgcccccaagcaggagcccaaagaccgcgagcccctgacctccctgaaatccctgtttggctctgaccccctgtcccagatggctggcaaatggtccaagtcctccattgtggggtggcctgctgtgcgcgagcggatccggcggacagagcctgctgctgagggcgtgggcgctgcctcccaggatctggacaagtatggcgccctgacctccagcaacacagctgccaacaatgctgactgtgcctggctggaagcccaggaggaagaagaagtgggctttcctgtgcggccccaggtgcccctgcggccaatgacctacaaagctgcctttgacctgtccttcttcctgaaagaaaaaggcggcctggaaggcctgatttactccaagaagcggcaggagatcctggacctgtgggtctaccacacccagggcttcttccctgattggcagaactacacccctgggcctggcgtgcggtaccccctgacctttgggtggtgcttcaaactggtgcctgtggacccccgcgaagtggaagaagccaatgaaggcgaaaacaactgcctgctgcatcccatgtcccagcatgggatggatgaccctgagaaagaagtgctggtctggaagttcgactcccggctggccttccaccacatggcccgggagctgcatcctgagtactataaagactgcatgggcgcccgcgcctccatcctgcgcggcggcaaactggacaagtgggagaagatccggctgaggcctggcggcaagaagcactacatgctgaagcatctcgtctgggcctcccgcgagctcgagcggtttgccctgaacccctccctgctggagacatctgaaggctgcaagcagatcatgaagcagctgcaacctgccctgcagacaggcacagaggagctgcggtccctgtacaacacagtggcgaccctgtactgcgtgcatcagcggattgatgtgaaagacaccaaagaagccctggacaaaattgaagaagagcagaacaagtccaagaagaaagcccagcaggctgctgctgacacaggcaactcctcccaggtgtcccagaattaccccattgtgcagaatgcccagggccaaatggtgcatcagcccctgtccccccggaccctgaacgcctgggtgaaagtggtggaagagaaaggcttcaaccctgaagtgattcccatgttcacagccctgtctgagggcgccaccccccaggatctgaacaccatgctgaacacagtgggcggccatcaggctgctatgcagatgctgaaagacaccattaatgaagaagctgctgaatgggaccgcgtgcatcctgtgcatgctggccccatcccccctggccaaatgcgcgagccccgcggctctgatattgctggcaccacctccaccccccaggagcaaattggctggatgacctccaacccccccattcctgtgggcgaaatctacaagcggtggatcatcatgggcctgaataagattgtgcggatgtactcccccgtgtccatcctggacatccggcagggccctaaagagccattccgcgactatgtcgaccggttcttcaagaccctgagagctgagcaggccacccaggaagtgaagaattggatgacagagaccctgctggtgcagaatgccaatcctgactgcaagtccatcctgaaagccctgggcacaggcgccaccctggaagaaatgatgacagcctgccagggggtgggcggccctggccacaaagcccgcgtgctggctgaggccatgtcccaggccaactccaacatcctgatgcagcggtccaacttcaaaggctccaagcggattgtgaagtgctttaactgtggcaaagaaggccacattgcccggaactgtcgcgccccccggaagaaaggctgttggaagtgtggccgcgaaggccatcagatgaaagactgtacagagcggcaggccaacttcctcggcaaaatctggccctcctccaaaggccggcccggcaacttcccccagtcccggcctgagcccacagccccccccgctgagtccttccggtttggcgaagagaccaccaccccctcccagaagcaggaacccattgacaaagagctgtaccccctggcctccctgaagtccctgtttggcaatgacccctcctcccagatggctggcaagtggtccaagtccagcattgtgggctggcctgccatccgcgagcggattcggcggacagaccctgctgctgaaggcgtgggcgccgcctcccaggacctggacaagcatggcgccctcacctcctccaacacagccaccaacaatgctgcctgtgcctggctcgaagcccaggaagaagaagaggtgggcttccctgtgaagccccaggtgcctctgcggcccatgacctataaaggcgccctggacctgtcccacttcctgaaagagaaaggcggcctcgaaggcctgatctactcccagaagcggcaggacatcctggacctctgggtctacaacacccagggctacttccctgactggcagaactatacccctggccctggcatccggtaccccctcacctttggctggtgcttcaagctggtgcccgtggaccctgatgaagtggagaaagccacagaaggcgagaacaactccctgctgcaccccatctgccagcatggcatggatgatgaagagaaagaggtgctgatgtggaagtttgactcctccctggcccggcggcacatggcccgcgagctgcatcccgagtactacaaagactgc

SEQ ID NO: 45 - DNA, 3357 bp, Artificial Sequence
  HIV-1 GAG(G1)NEF(B)NEF(G1)NEF(G2) expression cassette
atgggcgctcgcgcctctgtgctgtctggcggcaagctggatgcctgggagaagattcggctgcggcctggcgggaagaagaagtaccggctgaagcatctggtctgggccagccgcgagctggagcggtttgctctgaaccctggcctgctggagacctctgaaggctgtaagcagatcatcaagcagctgcagcctgccctgcaaacaggcacagaagagctgagatccctgttcaacacagtggccaccctgtactgtgtgcatgagaagattgaagtgcgcgacaccaaagaggccctggacaagattgaagaagaacagaacaagtcccagcagaagacccagcaggccaaagctgctgatggcaaagtgtcccagaactaccccatcgtgcagaacctgcagggccagatggtgcatcaggccctgtccccacggaccctgaatgcctgggtcaaagtgattgaagagaaagccttctcccctgaagtgatccccatgttcaccgccctgtctgaaggcgccacaccccaggacctgaacatgatgctgaacattgtgggcggccaccaggctgccatgcagatgctcaaagacaccatcaatgaagaggctgctgagtgggaccggctgcatcctgtccatgctggccctgtggcccctggccagatgcgcgagcctcgcggctctgacattgctggcacaacctccaccctgcaggagcagattgcctggatgaccagcaacccccccatccctgtgggcgacatctacaagagatggatcatcctgggcctgaacaagattgtgagaatgtactcccctgtgtccattctggacatcaagcagggccccaaagagccatttcgcgactatgtggaccggttctttaagaccctgcgcgctgagcaggctacccaggatgtgaagaactggatgacagacaccctgctggtccagaatgccaaccctgactgcaagaccatcctgcgcgccctgggccctggcgccaccctcgaagagatgatgacagcttgccagggcgtgggcggcccctcccacaaagccagagtgctggctgaagccatgagccaggcccagcacaccaacatcatgatgcagcgcggcaacttccgcggccagaagcggatcaagtgcttcaactgtggcaaggaaggccatctggcccggaactgccgcgcccccagaaagaaaggctgctggaagtgtggcaaagaaggccatcaaatgaaagactgcacagagcggcaagccaacttcctgggcaagatttggccctcccacaaaggccggcctggcaacttcctgcagaaccggcctgagcctacagccccccctgctgagagcttccggtttgaagagaccacccctgcccccaagcaggagcccaaagaccgcgagcccctgacctccctgaaatccctgtttggctctgaccccctgtcccagatggccggcaagtggagcaagaggtccgtgcccggctggtccaccgtgagggagaggatgaggagggccgagcccgccgccgacagggtgaggaggaccgagcccgccgcagtgggcgtcggggccgtgtccagggacctggagaagcacggcgccatcaccagctccaacaccgccgccaccaacgccgactgcgcctggctggaggcccaagaggacgaggaggtcggcttccccgtgaggccccaggtccccctgaggcccatgacatacaagggcgccgtggacctgagccacttcctcaaggagaagggcgggctggagggcctgatccactcccagaagaggcaggacatcctggatctgtgggtgtaccacactcagggctacttccccgactggcaaaactacacccccggccccggcatcaggttccccctgacattcggctggtgtttcaagctggtccccgtggagcccgagaaggtggaggaggccaacgagggcgagaacaactgtctgctgcaccccatgagccagcacggcatcgaggaccccgagaaggaggtgctggagtggaggttcgactccaagctggcctttcaccacgtggccagggagctgcaccccgagtactacaaggactgcatggctggcaaatggtccaagtcctccattgtggggtggcctgctgtgcgcgagcggatccggcggacagagcctgctgctgagggcgtgggcgctgcctcccaggatctggacaagtatggcgccctgacctccagcaacacagctgccaacaatgctgactgtgcctggctggaagcccaggaggaagaagaagtgggctttcctgtgcggccccaggtgcccctgcggccaatgacctacaaagctgcctttgacctgtccttcttcctgaaagaaaaaggcggcctggaaggcctgatttactccaagaagcggcaggagatcctggacctgtgggtctaccacacccagggcttcttccctgattggcagaactacacccctgggcctggcgtgcggtaccccctgacctttgggtggtgcttcaaactggtgcctgtggacccccgcgaagtggaagaagccaatgaaggcgaaaacaactgcctgctgcatcccatgtcccagcatgggatggatgaccctgagaaagaagtgctggtctggaagttcgactcccggctggccttccaccacatggcccgggagctgcatcctgagtactataaagactgcatggctgggaagtggtccaaatccagcattgtgggctggcctgccatccgcgaacggattcggcggacagaccctgccgctgaaggggtcggcgccgcctcccaggacctggacaagcatggcgccctcacctcctccaacacagccaccaacaacgctgcctgtgcttggctcgaagcccaggaagaagaagaggtgggcttccctgtgaagccccaagtgcctctgcggcccatgacctataaaggcgccctggacctgtcccactttctcaaagagaaagggggcctcgaaggcctgatctacagccagaagcggcaagacatcctcgacctctgggtctacaacacccaggggtacttccctgactggcagaactatacccctggccctggcatccggtacccactcacctttggctggtgctttaagctggtgcccgtggaccctgatgaagtggagaaagccacagaaggcgagaataactccctgctccaccccatctgccagcatggcatggatgatgaagagaaagaggtgctgatgtggaagtttgactcctccctggcccggcggcacatggcccgcgagctgcatcccgaatactacaaagattgc

SEQ ID NO: 46 - protein, 500 aa, Artificial Sequence
  HIV-1 CO gag
MGARASVLSGGELDKWEKIRLRPGGKKKYKLKHIVWASRELERFAVNPGLLETSEGCRQILGQLQPSLQTGSEELRSLYNTVATLYCVHQKIDVKDTKEALEKIEEEQNKSKKKAQQAAAGTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKVGHIAKNCRAPRKKGCWKCGKEGHQMKDCNERQANFLGKIWPSHKGRPGNFLQSRPEPTAPPEESFRFGEEKTTPSQKQEPIDKELYPLASLRSLFGNDPSSQ

SEQ ID NO: 47 - DNA, 1521 bp, Artificial Sequence
  codon optimized full-length HIV-1 p55 gag
atgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagtaaaataaagcccgggcagat

SEQ ID NO: 48 - protein, 12 aa, Artificial Sequence
  hypothetical example to illustrate sequence alignment
ACDEFGHIKLMN

SEQ ID NO: 49 - protein, 12 aa, Artificial Sequence
  hypothetical example to illustrate sequence alignment
ACDEHGHIKLMN

SEQ ID NO: 50 - protein, 12 aa, Artificial Sequence
  hypothetical example to hypothetical example to illustrate sequence alignment
ACDEWNHIKLMN

SEQ ID NO: 51 - protein, 12 aa, Artificial Sequence
  hypothetical example to illustrate sequence alignment
ACDEWLHIKLMN

SEQ ID NO: 52 - protein, 12 aa, Artificial Sequence
  sequence alignment of hypothetical examples
ACDEWGHIKLMN

SEQ ID NO: 53 - protein, 15 aa, Artificial Sequence
  hypothetical example to illustrate successive sequence fragments
ACDEFGHIKLMNRST

SEQ ID NO: 54 - protein, 9 aa, Artificial Sequence
  sequence fragment derived from hypothetical example to illustrate successive sequence fragments
ACDEFGHIK

SEQ ID NO: 55 - protein, 9 aa, Artificial Sequence
  sequence fragment derived from hypothetical example to illustrate successive sequence fragments
CDEFGHIKL

SEQ ID NO: 56 - protein, 9 aa, Artificial Sequence
  sequence fragment derived from hypothetical example to illustrate successive sequence fragments
DEFGHIKLM

SEQ ID NO: 57 - protein, 9 aa, Artificial Sequence
  sequence fragment derived from hypothetical example to illustrate successive sequence fragments
EFGHIKLMN

SEQ ID NO: 58 - protein, 9 aa, Artificial Sequence
  sequence fragment derived from hypothetical example to illustrate successive sequence fragments
FGHIKLMNR

SEQ ID NO: 59 - protein, 9 aa, Artificial Sequence
  sequence fragment derived from hypothetical example to illustrate successive sequence fragments
GHIKLMNRS

SEQ ID NO: 60 - protein, 9 aa, Artificial Sequence
  sequence fragment derived from hypothetical example to illustrate successive sequence fragments
HIKLMNRST

SEQ ID NO: 61 - protein, 1401 aa, Artificial Sequence
  HIV-1 GAG.N16.1-GAG.N16.2-NEF.N16.1-NEF.N16.2 fusion protein
MGARASVLSGGKLDAWEKIRLRPGGKKKYRLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQALSPRTLNAWVKVIEEKAFSPEVIPMFTALSEGATPQDLNMMLNIVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPSHKARVLAEAMSQAQHTNIMMQRGNFRGQKRIKCFNCGKEGHLARNCRAPRKKGCWKCGKEGHQMKDCTERQANFLGKIWPSHKGRPGNFLQNRPEPTAPPAESFRFEETTPAPKQEPKDREPLTSLKSLFGSDPLSQMGARASILRGGKLDKWEKIRLRPGGKKHYMLKHLVWASRELERFALNPSLLETSEGCKQIMKQLQPALQTGTEELRSLYNTVATLYCVHQRIDVKDTKEALDKIEEEQNKSKKKAQQAAADTGNSSQVSQNYPIVQNAQGQMVHQPLSPRTLNAWVKVVEEKGFNPEVIPMFTALSEGATPQDLNTMLNTVGGHQAAMQMLKDTINEEAAEWDRVHPVHAGPIPPGQMREPRGSDIAGTTSTPQEQIGWMTSNPPIPVGEIYKRWIIMGLNKIVRMYSPVSILDIRQGPKEPFRDYVDRFFKTLRAEQATQEVKNWMTETLLVQNANPDCKSILKALGTGATLEEMMTACQGVGGPGHKARVLAEAMSQANSNILMQRSNFKGSKRIVKCFNCGKEGHIARNCRAPRKKGCWKCGREGHQMKDCTERQANFLGKIWPSSKGRPGNFPQSRPEPTAPPAESFRFGEETTTPSQKQEPIDKELYPLASLKSLFGNDPSSQMGGKWSKSSIVGWPAVRERIRRTEPAAEGVGAASQDLDKYGALTSSNTAANNADCAWLEAQEEEEVGFPVRPQVPLRPMTYKAAFDLSFFLKEKGGLEGLIYSKKRQEILDLWVYHTQGFFPDWQNYTPGPGVRYPLTFGWCFKLVPVDPREVEEANEGENNCLLHPMSQHGMDDPEKEVLVWKFDSRLAFHHMARELHPEYYKDCMGGKWSKSSIVGWPAIRERIRRTDPAAEGVGAASQDLDKHGALTSSNTATNNAACAWLEAQEEEEVGFPVKPQVPLRPMTYKGALDLSHFLKEKGGLEGLIYSQKRQDILDLWVYNTQGYFPDWQNYTPGPGIRYPLTFGWCFKLVPVDPDEVEKATEGENNSLLHPICQHGMDDEEKEVLMWKFDSSLARRHMARELHPEYYKDC

SEQ ID NO: 62 - protein, 1401 aa, Artificial Sequence
  HIV-1 GAG.N16.1-NEF.N16.1-GAG.N16.2-NEF.N16.2 fusion protein
MGARASVLSGGKLDAWEKIRLRPGGKKKYRLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQALSPRTLNAWVKVIEEKAFSPEVIPMFTALSEGATPQDLNMMLNIVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPSHKARVLAEAMSQAQHTNIMMQRGNFRGQKRIKCFNCGKEGHLARNCRAPRKKGCWKCGKEGHQMKDCTERQANFLGKIWPSHKGRPGNFLQNRPEPTAPPAESFRFEETTPAPKQEPKDREPLTSLKSLFGSDPLSQMGGKWSKSSIVGWPAVRERIRRTEPAAEGVGAASQDLDKYGALTSSNTAANNADCAWLEAQEEEEVGFPVRPQVPLRPMTYKAAFDLSFFLKEKGGLEGLIYSKKRQEILDLWVYHTQGFFPDWQNYTPGPGVRYPLTFGWCFKLVPVDPREVEEANEGENNCLLHPMSQHGMDDPEKEVLVWKFDSRLAFHHMARELHPEYYKDCMGARASILRGGKLDKWEKIRLRPGGKKHYMLKHLVWASRELERFALNPSLLETSEGCKQIMKQLQPALQTGTEELRSLYNTVATLYCVHQRIDVKDTKEALDKIEEEQNKSKKKAQQAAADTGNSSQVSQNYPIVQNAQGQMVHQPLSPRTLNAWVKVVEEKGFNPEVIPMFTALSEGATPQDLNTMLNTVGGHQAAMQMLKDTINEEAAEWDRVHPVHAGPIPPGQMREPRGSDIAGTTSTPQEQIGWMTSNPPIPVGEIYKRWIIMGLNKIVRMYSPVSILDIRQGPKEPFRDYVDRFFKTLRAEQATQEVKNWMTETLLVQNANPDCKSILKALGTGATLEEMMTACQGVGGPGHKARVLAEAMSQANSNILMQRSNFKGSKRIVKCFNCGKEGHIARNCRAPRKKGCWKCGREGHQMKDCTERQANFLGKIWPSSKGRPGNFPQSRPEPTAPPAESFRFGEETTTPSQKQEPIDKELYPLASLKSLFGNDPSSQMGGKWSKSSIVGWPAIRERIRRTDPAAEGVGAASQDLDKHGALTSSNTATNNAACAWLEAQEEEEVGFPVKPQVPLRPMTYKGALDLSHFLKEKGGLEGLIYSQKRQDILDLWVYNTQGYFPDWQNYTPGPGIRYPLTFGWCFKLVPVDPDEVEKATEGENNSLLHPICQHGMDDEEKEVLMWKFDSSLARRHMARELHPEYYKDC

SEQ ID NO: 63 - protein, 1119 aa, Artificial Sequence
  HIV-1 GAG.N16.1-NEFJFRL-NEF.N16.1-NEF.N16.2 fusion protein
MGARASVLSGGKLDAWEKIRLRPGGKKKYRLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQALSPRTLNAWVKVIEEKAFSPEVIPMFTALSEGATPQDLNMMLNIVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPSHKARVLAEAMSQAQHTNIMMQRGNFRGQKRIKCFNCGKEGHLARNCRAPRKKGCWKCGKEGHQMKDCTERQANFLGKIWPSHKGRPGNFLQNRPEPTAPPAESFRFEETTPAPKQEPKDREPLTSLKSLFGSDPLSQMGGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDCMGGKWSKSSIVGWPAVRERIRRTEPAAEGVGAASQDLDKYGALTSSNTAANNADCAWLEAQEEEEVGFPVRPQVPLRPMTYKAAFDLSFFLKEKGGLEGLIYSKKRQEILDLWVYHTQGFFPDWQNYTPGPGVRYPLTFGWCFKLVPVDPREVEEANEGENNCLLHPMSQHGMDDPEKEVLVWKFDSRLAFHHMARELHPEYYKDCMGGKWSKSSIVGWPAIRERIRRTDPAAEGVGAASQDLDKHGALTSSNTATNNAACAWLEAQEEEEVGFPVKPQVPLRPMTYKGALDLSHFLKEKGGLEGLIYSQKRQDILDLWVYNTQGYFPDWQNYTPGPGIRYPLTFGWCFKLVPVDPDEVEKATEGENNSLLHPICQHGMDDEEKEVLMWKFDSSLARRHMARELHPEYYKDC

SEQ ID NO: 64 - protein, 486 aa, Artificial Sequence
  HIV-1 GAGglobal.N9 consensus sequence
MGARASVLSGGKLDAWEKIRLRPGGKKKYRLKHLVWASRELERFALNPGLLETSEGCKQIIKQLQPALQTGTEELRSLYNTVATLYCVHQRIDVKDTKEALDKIEEEQNKSQQKTQQAEKVSQNYPIVQNLQGQMVHQAISPRTLNAWVKVIEEKAFSPEVIPMFSALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIRQGPKEPFRDYVDRFFKTLRAEQATQEVKNWMTETLLVQNANPDCKTILKALGPAATLEEMMTACQGVGGPGHKARVLAEAMSQANSNIMMQRGNFKGQKRIKCFNCGKEGHIARNCRAPRKKGCWKCGKEGHQMKDCTERQANFLGKIWPSHKGRPGNFLQSRPEPTAPPAESFRFEETTPAPKQEPKDREPLTSLKSLFGSDPLSQ

SEQ ID NO: 65 - protein, 479 aa, Artificial Sequence
  HIV-1 GAGglobal.N9 delta(486) constrained consensus sequence
MGARASILRGGKLDKWEKIRLRPGGKKKYKLKHIVWASRELERFAVNPGLLETSEGCRQILGQLQPSLQTGSEELRSLFNTVATLYCVHQRIEVKDTKEALEKIEEEQNKSKKKAQQAAADTGNSSQVSQNYPIVQNAQGQMVHQALSPRTLNAWVKVVEEKAFSPEVIPMFTALSEGATPQDLNMMLNIVGGHQAAMQMLKETINEEAAEWDRVHPVHAGPVAPGQMRDPRGSDIAGSTSTLQEQIGWMTNNPPIPVGEIYKRWIILGLDKIVRMYSPTSILDIKQGPKEPFRDYVDRFYKTLRAEQATQDVKNWMTDTLLVQNANPDCKTILRALGPGATLEEMMTACQGVGGPSHKARVLAEAMSQVTNSATIMMQRGNFRNQRKTVKCFNCGKEGHQMKECTERQANFLGRIWPSSKGRPGNFLQNRPEPTAPPEESFRFGEETTTPSQKQEPIDKELYPLASLKSLFGNDPLSQ

SEQ ID NO: 66 - protein, 495 aa, Artificial Sequence
  HIV-1 GAGglobal.N9 delta(486, 479) constrained consensus sequence
MGARASVLSGGELDRWEKIRLRPGGKKHYMLKHLVWASRELDRFALNPGLLETAEGCQQIIEQLQSTLKTGSEELKSLYNTVATLYCVHEKIEVRDTKEALDKIEEIQNKSKQKTQQAKAADGKVSQNYPIVQNIQGQMVHQPISPRTLNAWVKVVEEKGFNPEVIPMFSALSEGATPSDLNTMLNTIGGHQAAMQILKDTINEEAAEWDRTHPVHAGPIPPGQMREPRGGDIAGTTSTPQEQIGWMTSNPPVPVGDIYKRWIIMGLNKIVRMHSPVSILDIKQGPKESFRDYVDRFLKTLRAEQASQEVKNWMTDTLLIQNANPDCKSILRALGPGASLEEMMTACQEVGGPSHKARILAEAMSQVQHTNIMMQRSNFKGPKRIVKCFNCGKEGHLARNCRAPRKKGCWKCGREGHQMKDCNERQANFLGKIWPSNKGRPGNFPQSRPEPTAPPAENWGMGEEITSPPKQEQKDKELYPLASLRSLFGNDPSSQ

SEQ ID NO: 67 - protein, 491 aa, Artificial Sequence
  HIV-1 GAGglobal.N9 delta(500) constrained consensus sequence
MGARASVLSGGKLDAWEKIRLRPGGKKHYMLKHLVWASRELERFALNPGLLETSEGCKQIIKQLQPALQTGTEELRSLFNTVATLYCVHQRIEVKDTKEALDKIEEEQNKSQQKTQQAKAADGKVSQNYPIVQNAQGQMVHQALSPRTLNAWVKVIEEKAFSPEVIPMFTALSEGATPQDLNMMLNIVGGHQAAMQMLKDTINEEAAEWDRVHPVHAGPVAPGQMRDPRGSDIAGSTSTLQEQIAWMTSNPPIPVGDIYKRWIILGLDKIVRMYSPVSILDIKQGPKEPFRDYVDRFFKTLRAEQATQDVKNWMTDTLLVQNANPDCKTILRALGPGATLEEMMTACQGVGGPSHKARVLAEAMSQANNTNIMMQRGNFKGQKRIKCFNCGKEGHIARNCRAPRKKGCWKCGREGHQMKDCTERQANFLGRIWPSSKGRPGNFLQNRPEPTAPPAESFRFEETTPAPKQEPKDREPLTSLKSLFGSDPLSQ

SEQ ID NO: 68 - protein, 499 aa, Artificial Sequence
  HIV-1 GAGglobal.N9 delta(500, 491) constrained consensus sequence
MGARASILRGGKLDKWERIRLRPGGKKKYRLKHLVWASRELDRFALNPGLLETAEGCQQIIEQLQSTLKTGSEELKSLYNTVATLYCVHEKIEVRDTKEALDKIEEIQNKSKQKTQQAAADTGNSSKVSQNYPIVQNIQGQMVHQPISPRTLNAWVKVVEEKGFNPEVIPMFSALSEGATPSDLNTMLNTIGGHQAAMQILKDTINEEAAEWDRTHPVHAGPIPPGQMREPRGGDIAGTTSTPQEQIGWMTSNPPVPVGDIYKRWIIMGLNKIVRMHSPVSILDIRQGPKESFRDYVDRFFKALRAEQATQEVKNWMTDTLLIQNANPDCKSILRALGPGASLEEMMTACQEVGGPSHKARILAEAMSQVQHTNIMMQRSNFKGPKRIVKCFNCGKEGHLARNCRAPRKRGCWKCGKEGHQMKECTERQANFLGKIWPSNKGRPGNFPQSRPEPSAPPAESFRFGEETTTPSQKQEQKDKELYPLASLKSLFGNDPLSQ

SEQ ID NO: 69 - protein, 492 aa, Artificial Sequence
  HIV-1 GAGglobal.N16 constrained consensus sequence
MGARASVLSGGKLDAWEKIRLRPGGKKKYRLKHLVWASRELERFALNPGLLETSEGCKQIIKQLQPALQTGTEELRSLYNTVATLYCVHEKIEVRDTKEALDKIEEEQNKSQQKTQQAKAADGKVSQNYPIVQNLQGQMVHQAISPRTLNAWVKVIEEKAFSPEVIPMFSALSEGATPQDLNTMLNTVGGHQAAMQMLKDTINEEAAEWDRLHPVHAGPIAPGQMREPRGSDIAGTTSTLQEQIGWMTNNPPIPVGEIYKRWIILGLNKIVRMYSPVSILDIKQGPKEPFRDYVDRFFKTLRAEQATQDVKNWMTDTLLVQNANPDCKTILKALGPAATLEEMMTACQGVGGPGHKARVLAEAMSQVTNSATIMMQRGNFRGQKRIKCFNCGKEGHLARNCRAPRKKGCWKCGKEGHQMKDCTERQANFLGKIWPSHKGRPGNFLQSRPEPTAPPAESFRFEETTPAPKQEPKDREPLTSLKSLFGSDPLSQ

SEQ ID NO: 70 - protein, 500 aa, Artificial Sequence
  HIV-1 GAGglobal.N16 delta(492) constrained consensus sequence
MGARASILRGGKLDKWEKIRLRPGGKKKYKLKHIVWASRELERFAVNPGLLETSEGCRQILGQLQPSLQTGSEELRSLFNTVATLYCVHQRIDVKDTKEALDKIEEEQNKSKKKAQQAAADTGNSSQVSQNYPIVQNLQGQMVHQALSPRTLNAWVKVVEEKAFSPEVIPMFTALSEGATPQDLNMMLNIVGGHQAAMQMLKETINEEAAEWDRLHPVHAGPVAPGQMREPRGSDIAGTTSTLQEQIAWMTSNPPIPVGDIYKRWIILGLNKIVRMYSPVSILDIRQGPKEPFRDYVDRFYKTLRAEQASQEVKNWMTETLLVQNANPDCKTILRALGPGATLEEMMTACQGVGGPSHKARVLAEAMSQATNSATIMMQRGNFRNQRKTVKCFNCGKEGHIARNCRAPRKKGCWKCGREGHQMKDCTERQANFLGKIWPSNKGRPGNFLQSRPEPTAPPEESFRFGEETTTPSQKQEPIDKELYPLASLRSLFGNDPSSQ

SEQ ID NO: 71 - protein, 496 aa, Artificial Sequence
  HIV-1 GAGglobal.N16 delta(492, 500) constrained consensus sequence
MGARASVLSGGELDRWEKIRLRPGGKKHYMLKHLVWASRELERFALNPSLLETSEGCKQIMKQLQPALQTGTEELKSLYNTVATLYCVHQRIEVKDTKEALDKIEEEQNKCQQKTQQAEAADKGKVSQNYPIVQNLQGQMVHQPISPRTLNAWVKVVEEKGFNPEVIPMFTALSEGATPQDLNTMLNTVGGHQAAMQMLKETINEEAAEWDRVHPVHAGPIPPGQMREPRGSDIAGTTSNLQEQIAWMTSNPPVPVGDIYKRWIILGLNKIVRMYSPTSILDIRQGPKEPFRDYVDRFFKTLRAEQATQEVKNWMTETLLVQNANPDCKSILKALGTGATLEEMMTACQGVGGPGHKARVLAEAMSQANSNILMQRSNFKGSKRIVKCFNCGKEGHIAKNCRAPRKKGCWKCGKEGHQMKECTERQANFLGKIWPSSKGRPGNFPQSRPEPTAPPAESFRFGEETTTPPQKQEPIDKELYPLASLKSLFGNDPSSQ

SEQ ID NO: 72 - protein, 498 aa, Artificial Sequence
  HIV-1 GAGglobal.N30 constrained consensus sequence
MGARASVLSGGELDRWEKIRLRPGGKKKYKLKHIVWASRELERFAVNPGLLETSEGCRQILGQLQPSLQTGSEELRSLYNTVATLYCVHQKIEVKDTKEALDKIEEEQNKSKKKAQQAAADTGNSSQVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPGHKARVLAEAMSQVQHTNIMMQRGNFRGQKRIKCFNCGKEGHLARNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQ

SEQ ID NO: 73 - protein, 493 aa, Artificial Sequence
  HIV-1 GAGglobal.N30 delta(498) constrained consensus sequence
MGARASILRGGKLDKWEKIRLRPGGKKHYMLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQNRPEPTAPPAESFRFEETTPAPKQEPKDREPLTSLKSLFGSDPLSQ

SEQ ID NO: 74 - protein, 496 aa, Artificial Sequence
  HIV-1 GAGglobal.N30 delta(498, 493) constrained consensus sequence
MGARASVLSGGKLDAWEKIRLRPGGKKKYRLKHLVWASRELERFALNPGLLETSEGCKQIMKQLQPALQTGTEELRSLFNTVATLYCVHAGIEVRDTKEALDKIEEEQNKSQQKTQQAKEADGKVSQNYPIVQNLQGQMVHQALSPRTLNAWVKVIEEKAFSPEVIPMFSALSEGATPQDLNMMLNIVGGHQAAMQMLKDTINEEAAEWDRLHPVHAGPVAPGQMREPRGSDIAGTTSTLQEQIGWMTSNPPIPVGEIYKRWIILGLNKIVRMYSPTSILDIRQGPKEPFRDYVDRFYKTLRAEQATQEVKNWMTETLLVQNANPDCKSILKALGTGATLEEMMTACQGVGGPSHKARVLAEAMSQANNTNIMMQKSNFKGSKRIVKCFNCGKEGHIARNCRAPRKKGCWKCGKEGHQMKDCTERQANFLGKIWPSSKGRPGNFPQSRPEPTAPPAEIFGMGEEITSPPKQEQKEREQTPPFVSLKSLFGNDPLSQ

SEQ ID NO: 75 - protein, 491 aa, Artificial Sequence
  HIV-1 GAGglobal.N30 delta(500) constrained consensus sequence
MGARASILRGGKLDKWEKIRLRPGGKKHYMLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPGHKARVLAEAMSQVQHTNIMMQRGNFRGQKRIKCFNCGKEGHLARNCRAPRKKGCWKCGKEGHQMKDCTERQANFLGKIWPSHKGRPGNFLQSRPEPTAPPAESFRFEETTPAPKQEPKDREPLTSLKSLFGSDPLSQ

SEQ ID NO: 76 - protein, 491 aa, Artificial Sequence
  HIV-1 GAGglobal.N30 delta(500, 491) constrained consensus sequence
MGARASVLSGGKLDAWEKIRLRPGGKKKYRLKHLVWASRELERFALNPGLLETSEGCKQIMKQLQPALQTGTEELRSLFNTVATLYCVHAGIEVRDTKEALDKIEEEQNKSQQKTQQAKEADGKVSQNYPIVQNLQGQMVHQALSPRTLNAWVKVIEEKAFSPEVIPMFSALSEGATPQDLNMMLNIVGGHQAAMQMLKDTINEEAAEWDRLHPVHAGPIPPGQMREPRGSDIAGTTSTLQEQIGWMTSNPPIPVGEIYKRWIILGLNKIVRMYSPVSILDIRQGPKEPFRDYVDRFFKTLRAEQATQEVKNWMTDTLLVQNANPDCKTILRALGPGASLEEMMTACQGVGGPGHKARVLAEAMSQANSNIMMQRGNFKGSKRIVKCFNCGKEGHIARNCRAPRKKGCWKCGKEGHQMKDCTERQANFLGKIWPSHKGRPGNFLQNRPEPTAPPAESFRFEETTPAPKQEPKDREPLISLKSLFGSDPLSQ

SEQ ID NO: 77 - protein, 173 aa, Artificial Sequence
  HIV-1 NEFglobal.N9 constrained consensus sequence
MGGKWSKSSIVGWPAVRERIRRTEPAAEGVGAVSRDLEKHGAITSSNTAATNADCAWLEAQEEEEVGFPVRPQVPLRPMTYKGALDLSHFLKEKGGLEGLIYSKKRQEILDLWVYHTQGYFPDWQNYTPGPGVRYPLTFGWCFKLVPVDPREVEEANEGENNCLLHPMSQHGM

SEQ ID NO: 78 - protein, 206 aa, Artificial Sequence
  HIV-1 NEFglobal.N9 delta(173) constrained consensus sequence
MGGKWSKSSVVGWPAVRERMRRAEPAAEGVGAASQDLDKHGALTSSNTAANNAACAWLEAQEDEEVGFPVKPQVPLRPMTYKAAFDLSFFLKEKGGLDGLIYSQKRQDILDLWVYNTQGFFPDWQNYTPGPGIRYPLTFGWCYKLVPVDPKEVEEANEGENNSLLHPMSLHGMDDPEREVLMWKFDSRLAFHHMARELHPEYYKDC

SEQ ID NO: 79 - protein, 198 aa, Artificial Sequence
  HIV-1 NEFglobal.N9 delta(173, 206) constrained consensus sequence
MGGKWSKSSIVGWPAIRERMRRTEPAADGVGAVSQDLDKYGALTSSNTPANNADCAWLQAQEEEEEVGFPMTYKAAVDLSHFLKEEGGLDGLIYSKKRQDILDLWVYHTQGFFPDWHNYTPGPGTRFPLTFGWCFKLVPVEPEKVEEATEGENNSLLHPICQHGMDDPEKEVLVWKFDSSLARRHMARELHPEFYKDC

SEQ ID NO: 80 - protein, 216 aa, Artificial Sequence
  HIV-1 NEF from JRFL isolate
MGGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDC

SEQ ID NO: 81 - protein, 173 aa, Artificial Sequence
  HIV-1 NEFglobal.N9 delta(216) constrained consensus sequence
MGGKWSKSSIVGWPAVRERIRRTEPAAEGVGAASQDLDKHGALTSSNTAANNAACAWLEAQEEEEVGFPVKPQVPLRPMTYKAAFDLSFFLKEKGGLDGLIYSKKRQEILDLWVYNTQGFFPDWQNYTPGPGVRYPLTFGWCFKLVPVDPREVEEANEGENNSLLHPMSQHGM

SEQ ID NO: 82 - protein, 207 aa, Artificial Sequence
  HIV-1 NEFglobal.N9 delta(216, 173) constrained consensus sequence
MGGKWSKSSVVGWPAVRERMRRTEPAAEGVGAVSQDLDKYGALTSSNTPANNADCAWLQAQEEEEEVGFPVRPQVPVRPMTYKGALDLSHFLREKGGLEGLIYSKKRQDILDLWVYHTQGFFPDWHNYTPGPGIRYPLTFGWCYKLVPVDPKEVEEANKGENNCLLHPMSLHGMDDPEREVLMWKFDSRLAFHHMARELHPEFYKDC

SEQ ID NO: 83 - protein, 206 aa, Artificial Sequence
  HIV-1 NEFglobal.N16 constrained consensus sequence
MGGKWSKSSIVGWPAVRERIRRTEPAAEGVGAVSRDLEKHGAITSSNTAATNADCAWLEAQEEEEVGFPVRPQVPLRPMTYKGALDLSHFLKEKGGLEGLIYSKKRQEILDLWVYHTQGYFPDWQNYTPGPGVRYPLTFGWCFKLVPVDPREVEEANEGENNCLLHPMSQHGMDDPEKEVLVWKFDSRLAFHHMARELHPEYYKDC

SEQ ID NO: 84 - protein, 207 aa, Artificial Sequence
  HIV-1 NEFglobal.N16 delta(206) constrained consensus sequence
MGGKWSKSSIVGWPAIRERIRRTEPAAEGVGAASQDLDKYGALTSSNTAANNADCAWLEAQEEEEEVGFPVRPQVPLRPMTYKAAVDLSHFLKEKGGLEGLVYSQKRQDILDLWVYHTQGFFPDWQNYTPGPGIRYPLTFGWCFKLVPVEPEKVEEANEGENNSLLHPMSLHGMDDPEKEVLMWKFDSRLAFHHMAREKHPEYYKDC

SEQ ID NO: 85 - protein, 206 aa, Artificial Sequence
  HIV-1 NEFglobal.N16 delta(206, 207) constrained consensus sequence
MGGKWSKSSIVGWPAVRERMRRTEPAAEGVGAASQDLDKHGALTSSNTATNNAACAWLEAQEEEEVGFPVKPQVPLRPMTYKGAFDLSFFLKEKGGLDGLIYSKKRQEILDLWVYNTQGYFPDWQNYTPGPGTRFPLTFGWCFKLVPVDPDEVEKATEGENNSLLHPICQHGMDDEEKEVLMWKFDSSLARRHMARELHPEYYKDC

SEQ ID NO: 86 - protein, 206 aa, Artificial Sequence
  HIV-1 NEFglobal.N30 constrained consensus sequence
MGGKWSKSSKIGWPTVRERMRRAEPAADGVGAVSRDLEKHGAITSSNTAATNADCAWLEAQEEEEVGFPVRPQVPLRPMTYKGAFDLSFFLKEKGGLEGLIYSKKRQEILDLWVYHTQGYFPDWQNYTPGPGVRYPLTFGWCFKLVPVDPREVEEANEGENNCLLHPMSQHGMEDEDREVLKWKFDSHLAREHIARQLHPEYYKDC

SEQ ID NO: 87 - protein, 206 aa, Artificial Sequence
  HIV-1 NEFglobal.N30 delta(206) constrained consensus sequence
MGGKWSKRGVPGWNTIRERMRRTEPAAEGVGAVSRDLEQRGAITTSNTASNNAACAWLEAQEEEEVGFPVRPQVPLRPMTYKGALDLSHFLKEKGGLEGLIYSQKRQDILDLWVYHTQGYFPDWQNYTPGPGIRYPLTFGWCFKLVPVDPDEVEKATEGENNSLLHPICQHGMDDEEKEVLMWKFDSRLALTHRARELHPEFYKDC

SEQ ID NO: 88 - protein, 207 aa, Artificial Sequence
  HIV-1 NEFglobal.N30 delta(206, 206) constrained consensus sequence
MGGKWSKSSIVGWPQVRERIRRAPAPAARGVGPVSQDLDKHGAVTSSNTAANNADCAWLEAQEEEEVGFPVRPQVPLRPMTYKAAVDLSHFLKEKGGLEGLIYSKKRQEILDLWVYHTQGFFPDWQNYTPGPGVRYPLTFGWCFKLVPVEPEKVEEATVGENNCLLHPMNLHGMDDPEGEVLVWKFDSRLAFHHMAREKHPEYYKDC

SEQ ID NO: 89 - protein, 206 aa, Artificial Sequence; identical to SEQ ID NO: 86
  HIV-1 NEFglobal.N30 delta(216) constrained consensus sequence

SEQ ID NO: 90 - protein, 206 aa, Artificial Sequence; identical to SEQ ID NO: 87
  HIV-1 NEFglobal.N30 delta(216, 206) constrained consensus sequence

SEQ ID NO: 91 - protein, 492 aa, Artificial Sequence
  HIV-1 Clade C Consensus Sequence
MGARASILRGGKLDKWEKIRLRPGGKKHYMLKHLVWASRELERFALNPGLLETSEGCKQIIKQLQPALQTGTEELRSLYNTVATLYCVHEGIEVRDTKEALDKIEEEQNKSQQKTQQAKAADGKVSQNYPIVQNLQGQMVHQAISPRTLNAWVKVIEEKAFSPEVIPMFTALSEGATPQDLNTMLNTVGGHQAAMQMLKDTINEEAAEWDRLHPVHAGPIAPGQMREPRGSDIAGTTSTLQEQIAWMTSNPPVPVGDIYKRWIILGLNKIVRMYSPVSILDIKQGPKEPFRDYVDRFFKTLRAEQATQDVKNWMTDTLLVQNANPDCKTILRALGPGASLEEMMTACQGVGGPSHKARVLAEAMSQANNTNIMMQRSNFKGPKRIVKCFNCGKEGHIARNCRAPRKKGCWKCGKEGHQMKDCTERQANFLGKIWPSHKGRPGNFLQSRPEPTAPPAESFRFEETTPAPKQEPKDREPLTSLKSLFGSDPLSQ

SEQ ID NO: 92 - protein, 206 aa, Artificial Sequence
  HIV-1 optimized NEFglobal
MAGKWSKSSIVGWPAVRERIRRTEPAAEGVGAASQDLDKYGALTSSNTAANNADCAWLEAQEEEEVGFPVRPQVPLRPMTYKAAFDLSFFLKEKGGLEGLIYSKKRQEILDLWVYHTQGFFPDWQNYTPGPGVRYPLTFGWCFKLVPVDPREVEEANEGENNCLLHPMSQHGMDDPEKEVLVWKFDSRLAFHHMARELHPEYYKDC

SEQ ID NO: 93 - protein, 206 aa, Artificial Sequence
  HIV-1 optimized NEFglobal#2
MAGKWSKSSIVGWPAIRERIRRTDPAAEGVGAASQDLDKHGALTSSNTATNNAACAWLEAQEEEEVGFPVKPQVPLRPMTYKGALDLSHFLKEKGGLEGLIYSQKRQDILDLWVYNTQGYFPDWQNYTPGPGIRYPLTFGWCFKLVPVDPDEVEKATEGENNSLLHPICQHGMDDEEKEVLMWKFDSSLARRHMARELHPEYYKDC

SEQ ID NO: 94 - protein, 1401 aa, Artificial Sequence
  HIV-1 optimized GAGGAGNEFNEF fusion protein
MGARASVLSGGKLDAWEKIRLRPGGKKKYRLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQALSPRTLNAWVKVIEEKAFSPEVIPMFTALSEGATPQDLNMMLNIVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPSHKARVLAEAMSQAQHTNIMMQRGNFRGQKRIKCFNCGKEGHLARNCRAPRKKGCWKCGKEGHQMKDCTERQANFLGKIWPSHKGRPGNFLQNRPEPTAPPAESFRFEETTPAPKQEPKDREPLTSLKSLFGSDPLSQMGARASILRGGKLDKWEKIRLRPGGKKHYMLKHLVWASRELERFALNPSLLETSEGCKQIMKQLQPALQTGTEELRSLYNTVATLYCVHQRIDVKDTKEALDKIEEEQNKSKKKAQQAAADTGNSSQVSQNYPIVQNAQGQMVHQPLSPRTLNAWVKVVEEKGFNPEVIPMFTALSEGATPQDLNTMLNTVGGHQAAMQMLKDTINEEAAEWDRVHPVHAGPIPPGQMREPRGSDIAGTTSTPQEQIGWMTSNPPIPVGEIYKRWIIMGLNKIVRMYSPVSILDIRQGPKEPFRDYVDRFFKTLRAEQATQEVKNWMTETLLVQNANPDCKSILKALGTGATLEEMMTACQGVGGPGHKARVLAEAMSQANSNILMQRSNFKGSKRIVKCFNCGKEGHIARNCRAPRKKGCWKCGREGHQMKDCTERQANFLGKIWPSSKGRPGNFPQSRPEPTAPPAESFRFGEETTTPSQKQEPIDKELYPLASLKSLFGNDPSSQMAGKWSKSSIVGWPAVRERIRRTEPAAEGVGAASQDLDKYGALTSSNTAANNADCAWLEAQEEEEVGFPVRPQVPLRPMTYKAAFDLSFFLKEKGGLEGLIYSKKRQEILDLWVYHTQGFFPDWQNYTPGPGVRYPLTFGWCFKLVPVDPREVEEANEGENNCLLHPMSQHGMDDPEKEVLVWKFDSRLAFHHMARELHPEYYKDCMAGKWSKSSIVGWPAIRERIRRTDPAAEGVGAASQDLDKHGALTSSNTATNNAACAWLEAQEEEEVGFPVKPQVPLRPMTYKGALDLSHFLKEKGGLEGLIYSQKRQDILDLWVYNTQGYFPDWQNYTPGPGIRYPLTFGWCFKLVPVDPDEVEKATEGENNSLLHPICQHGMDDEEKEVLMWKFDSSLARRHMARELHPEYYKDC

SEQ ID NO: 95 - protein, 1119 aa, Artificial Sequence
  HIV-1 optimized GAGNEFNEFNEF fusion protein
MGARASVLSGGKLDAWEKIRLRPGGKKKYRLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQALSPRTLNAWVKVIEEKAFSPEVIPMFTALSEGATPQDLNMMLNIVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPSHKARVLAEAMSQAQHTNIMMQRGNFRGQKRIKCFNCGKEGHLARNCRAPRKKGCWKCGKEGHQMKDCTERQANFLGKIWPSHKGRPGNFLQNRPEPTAPPAESFRFEETTPAPKQEPKDREPLTSLKSLFGSDPLSQMAGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDCMAGKWSKSSIVGWPAVRERIRRTEPAAEGVGAASQDLDKYGALTSSNTAANNADCAWLEAQEEEEVGFPVRPQVPLRPMTYKAAFDLSFFLKEKGGLEGLIYSKKRQEILDLWVYHTQGFFPDWQNYTPGPGVRYPLTFGWCFKLVPVDPREVEEANEGENNCLLHPMSQHGMDDPEKEVLVWKFDSRLAFHHMARELHPEYYKDCMAGKWSKSSIVGWPAIRERIRRTDPAAEGVGAASQDLDKHGALTSSNTATNNAACAWLEAQEEEEVGFPVKPQVPLRPMTYKGALDLSHFLKEKGGLEGLIYSQKRQDILDLWVYNTQGYFPDWQNYTPGPGIRYPLTFGWCFKLVPVDPDEVEKATEGENNSLLHPICQHGMDDEEKEVLMWKFDSSLARRHMARELHPEYYKDC

SEQ ID NO: 96 - protein, 1401 aa, Artificial Sequence
  HIV-1 optimized GAGNEFGAGNEF fusion protein
MGARASVLSGGKLDAWEKIRLRPGGKKKYRLKHLVWASRELERFALNPGLLETSEGCKQIIKQLQPALQTGTEELRSLFNTVATLYCVHEKIEVRDTKEALDKIEEEQNKSQQKTQQAKAADGKVSQNYPIVQNLQGQMVHQALSPRTLNAWVKVIEEKAFSPEVIPMFTALSEGATPQDLNMMLNIVGGHQAAMQMLKDTINEEAAEWDRLHPVHAGPVAPGQMREPRGSDIAGTTSTLQEQIAWMTSNPPIPVGDIYKRWIILGLNKIVRMYSPVSILDIKQGPKEPFRDYVDRFFKTLRAEQATQDVKNWMTDTLLVQNANPDCKTILRALGPGATLEEMMTACQGVGGPSHKARVLAEAMSQAQHTNIMMQRGNFRGQKRIKCFNCGKEGHLARNCRAPRKKGCWKCGKEGHQMKDCTERQANFLGKIWPSHKGRPGNFLQNRPEPTAPPAESFRFEETTPAPKQEPKDREPLTSLKSLFGSDPLSQMAGKWSKSSIVGWPAVRERIRRTEPAAEGVGAASQDLDKYGALTSSNTAANNADCAWLEAQEEEEVGFPVRPQVPLRPMTYKAAFDLSFFLKEKGGLEGLIYSKKRQEILDLWVYHTQGFFPDWQNYTPGPGVRYPLTFGWCFKLVPVDPREVEEANEGENNCLLHPMSQHGMDDPEKEVLVWKFDSRLAFHHMARELHPEYYKDCMGARASILRGGKLDKWEKIRLRPGGKKHYMLKHLVWASRELERFALNPSLLETSEGCKQIMKQLQPALQTGTEELRSLYNTVATLYCVHQRIDVKDTKEALDKIEEEQNKSKKKAQQAAADTGNSSQVSQNYPIVQNAQGQMVHQPLSPRTLNAWVKVVEEKGFNPEVIPMFTALSEGATPQDLNTMLNTVGGHQAAMQMLKDTINEEAAEWDRVHPVHAGPIPPGQMREPRGSDIAGTTSTPQEQIGWMTSNPPIPVGEIYKRWIIMGLNKIVRMYSPVSILDIRQGPKEPFRDYVDRFFKTLRAEQATQEVKNWMTETLLVQNANPDCKSILKALGTGATLEEMMTACQGVGGPGHKARVLAEAMSQANSNILMQRSNFKGSKRIVKCFNCGKEGHIARNCRAPRKKGCWKCGREGHQMKDCTERQANFLGKIWPSSKGRPGNFPQSRPEPTAPPAESFRFGEETTTPSQKQEPIDKELYPLASLKSLFGNDPSSQMAGKWSKSSIVGWPAIRERIRRTDPAAEGVGAASQDLDKHGALTSSNTATNNAACAWLEAQEEEEVGFPVKPQVPLRPMTYKGALDLSHFLKEKGGLEGLIYSQKRQDILDLWVYNTQGYFPDWQNYTPGPGIRYPLTFGWCFKLVPVDPDEVEKATEGENNSLLHPICQHGMDDEEKEVLMWKFDSSLARRHMARELHPEYYKDC

SEQ ID NO: 97 - protein, 173 aa, Artificial Sequence
  HIV-1 optimized NEFglobal.N9 constrained consensus sequence
MAGKWSKSSIVGWPAVRERIRRTEPAAEGVGAVSRDLEKHGAITSSNTAATNADCAWLEAQEEEEVGFPVRPQVPLRPMTYKGALDLSHFLKEKGGLEGLIYSKKRQEILDLWVYHTQGYFPDWQNYTPGPGVRYPLTFGWCFKLVPVDPREVEEANEGENNCLLHPMSQHGM

SEQ ID NO: 98 - protein, 206 aa, Artificial Sequence
  HIV-1 optimized NEFglobal.N9 delta(173) constrained consensus sequence
MAGKWSKSSVVGWPAVRERMRRAEPAAEGVGAASQDLDKHGALTSSNTAANNAACAWLEAQEDEEVGFPVKPQVPLRPMTYKAAFDLSFFLKEKGGLDGLIYSQKRQDILDLWVYNTQGFFPDWQNYTPGPGIRYPLTFGWCYKLVPVDPKEVEEANEGENNSLLHPMSLHGMDDPEREVLMWKFDSRLAFHHMARELHPEYYKDC

SEQ ID NO: 99 - protein, 198 aa, Artificial Sequence
  HIV-1 optimized NEFglobal.N9 delta(173, 206) constrained consensus sequence
MAGKWSKSSIVGWPAIRERMRRTEPAADGVGAVSQDLDKYGALTSSNTPANNADCAWLQAQEEEEEVGFPMTYKAAVDLSHFLKEEGGLDGLIYSKKRQDILDLWVYHTQGFFPDWHNYTPGPGTRFPLTFGWCFKLVPVEPEKVEEATEGENNSLLHPICQHGMDDPEKEVLVWKFDSSLARRHMARELHPEFYKDC

SEQ ID NO: 100 - protein, 216 aa, Artificial Sequence
  optimized HIV-1 NEF from JRFL isolate
MAGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDC

SEQ ID NO: 101 - protein, 173 aa, Artificial Sequence
  HIV-1 optimized NEFglobal.N9 delta(216) constrained consensus sequence
MAGKWSKSSIVGWPAVRERIRRTEPAAEGVGAASQDLDKHGALTSSNTAANNAACAWLEAQEEEEVGFPVKPQVPLRPMTYKAAFDLSFFLKEKGGLDGLIYSKKRQEILDLWVYNTQGFFPDWQNYTPGPGVRYPLTFGWCFKLVPVDPREVEEANEGENNSLLHPMSQHGM

SEQ ID NO: 102 - protein, 207 aa, Artificial Sequence
  HIV-1 optimized NEFglobal.N9 delta(216, 173) constrained consensus sequence
MAGKWSKSSVVGWPAVRERMRRTEPAAEGVGAVSQDLDKYGALTSSNTPANNADCAWLQAQEEEEEVGFPVRPQVPVRPMTYKGALDLSHFLREKGGLEGLIYSKKRQDILDLWVYHTQGFFPDWHNYTPGPGIRYPLTFGWCYKLVPVDPKEVEEANKGENNCLLHPMSLHGMDDPEREVLMWKFDSRLAFHHMARELHPEFYKDC

SEQ ID NO: 103 - protein, 206 aa, Artificial Sequence
  HIV-1 optimized NEFglobal.N16 constrained consensus sequence
MAGKWSKSSIVGWPAVRERIRRTEPAAEGVGAVSRDLEKHGAITSSNTAATNADCAWLEAQEEEEVGFPVRPQVPLRPMTYKGALDLSHFLKEKGGLEGLIYSKKRQEILDLWVYHTQGYFPDWQNYTPGPGVRYPLTFGWCFKLVPVDPREVEEANEGENNCLLHPMSQHGMDDPEKEVLVWKFDSRLAFHHMARELHPEYYKDC

SEQ ID NO: 104 - protein, 207 aa, Artificial Sequence
  HIV-1 optimized NEFglobal.N16 delta(206) constrained consensus sequence
MAGKWSKSSIVGWPAIRERIRRTEPAAEGVGAASQDLDKYGALTSSNTAANNADCAWLEAQEEEEEVGFPVRPQVPLRPMTYKAAVDLSHFLKEKGGLEGLVYSQKRQDILDLWVYHTQGFFPDWQNYTPGPGIRYPLTFGWCFKLVPVEPEKVEEANEGENNSLLHPMSLHGMDDPEKEVLMWKFDSRLAFHHMAREKHPEYYKDC

SEQ ID NO: 105 - protein, 206 aa, Artificial Sequence
  HIV-1 optimized NEFglobal.N16 delta(206, 207) constrained consensus sequence
MAGKWSKSSIVGWPAVRERMRRTEPAAEGVGAASQDLDKHGALTSSNTATNNAACAWLEAQEEEEVGFPVKPQVPLRPMTYKGAFDLSFFLKEKGGLDGLIYSKKRQEILDLWVYNTQGYFPDWQNYTPGPGTRFPLTFGWCFKLVPVDPDEVEKATEGENNSLLHPICQHGMDDEEKEVLMWKFDSSLARRHMARELHPEYYKDC

SEQ ID NO: 106 - protein, 206 aa, Artificial Sequence
  HIV-1 optimized NEFglobal.N30 constrained consensus sequence
MAGKWSKSSKIGWPTVRERMRRAEPAADGVGAVSRDLEKHGAITSSNTAATNADCAWLEAQEEEEVGFPVRPQVPLRPMTYKGAFDLSFFLKEKGGLEGLIYSKKRQEILDLWVYHTQGYFPDWQNYTPGPGVRYPLTFGWCFKLVPVDPREVEEANEGENNCLLHPMSQHGMEDEDREVLKWKFDSHLAREHIARQLHPEYYKDC

SEQ ID NO: 107 - protein, 206 aa, Artificial Sequence
  HIV-1 optimized NEFglobal.N30 delta(206) constrained consensus sequence
MAGKWSKRGVPGWNTIRERMRRTEPAAEGVGAVSRDLEQRGAITTSNTASNNAACAWLEAQEEEEVGFPVRPQVPLRPMTYKGALDLSHFLKEKGGLEGLIYSQKRQDILDLWVYHTQGYFPDWQNYTPGPGIRYPLTFGWCFKLVPVDPDEVEKATEGENNSLLHPICQHGMDDEEKEVLMWKFDSRLALTHRARELHPEFYKDC

SEQ ID NO: 108 - protein, 207 aa, Artificial Sequence
  HIV-1 optimized NEFglobal.N30 delta(206, 206) constrained consensus sequence
MAGKWSKSSIVGWPQVRERIRRAPAPAARGVGPVSQDLDKHGAVTSSNTAANNADCAWLEAQEEEEVGFPVRPQVPLRPMTYKAAVDLSHFLKEKGGLEGLIYSKKRQEILDLWVYHTQGFFPDWQNYTPGPGVRYPLTFGWCFKLVPVEPEKVEEATVGENNCLLHPMNLHGMDDPEGEVLVWKFDSRLAFHHMAREKHPEYYKDC

SEQ ID NO: 109 - protein, 206 aa, Artificial Sequence; identical to SEQ ID NO: 106
  HIV-1 optimized NEFglobal.N30 delta(216) constrained consensus sequence

SEQ ID NO: 110 - protein, 206 aa, Artificial Sequence; identical to SEQ ID NO: 107
  HIV-1 optimized NEFglobal.N30 delta(216, 206) constrained consensus sequence

SEQ ID NO: 111 - DNA, 2550 bp, Artificial Sequence
  HIV-1 pol
atggcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggctgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggctgccctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggctggggctgccaacagggagaccaagctgggcaaggctggctatgtgaccaacaggggcaggcagaaggtggtgaccctgactgacaccaccaaccagaagactgccctccaggccatctacctggccctccaggactctggcctggaggtgaacattgtgactgcctcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggcctgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgccaatggctccaacttcactggggccacagtgagggctgcctgctggtgggctggcatcaagcaggagtttggcatcccctacaacccccagtcccagggggtggtggcctccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggac

SEQ ID NO: 112 - protein, 850 aa, Artificial Sequence
  HIV-1 optimized Nef JRFL (G2A)
MAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVAGAANRETKLGKAGYVTNRGRQKVVTLTDTTNQKTALQAIYLALQDSGLEVNIVTASQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLACTHLEGKVILVAVHVASGYIEAEVIPAETGQETAYFLLKLAGRWPVKTIHTANGSNFTGATVRAACWWAGIKQEFGIPYNPQSQGVVASMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 113 - DNA, 648 bp, Artificial Sequence
  HIV-1 optimized Nef JRFL (G2A)
atggccggcaagtggagcaagaggtccgtgcccggctggtccaccgtgagggagaggatgaggagggccgagcccgccgccgacagggtgaggaggaccgagcccgccgcagtgggcgtcggggccgtgtccagggacctggagaagcacggcgccatcaccagctccaacaccgccgccaccaacgccgactgcgcctggctggaggcccaagaggacgaggaggtcggcttccccgtgaggccccaggtccccctgaggcccatgacatacaagggcgccgtggacctgagccacttcctcaaggagaagggcgggctggagggcctgatccactcccagaagaggcaggacatcctggatctgtgggtgtaccacactcagggctacttccccgactggcaaaactacacccccggccccggcatcaggttccccctgacattcggctggtgtttcaagctggtccccgtggagcccgagaaggtggaggaggccaacgagggcgagaacaactgtctgctgcaccccatgagccagcacggcatcgaggaccccgagaaggaggtgctggagtggaggttcgactccaagctggcctttcaccacgtggccagggagctgcaccccgagtactacaaggactgc

SEQ ID NO: 114 - DNA, 49 bp, Artificial Sequence
  short synthetic poly A signal for transcription termination
aataaaagatctttattttcattagatctgtgtgttggttttttgtgtg

(19) United States
(12) Patent Application Publication
(10) Pub. No.: US 2010/0183651 A1
(43) Pub. Date: Jul. 22, 2010

(54) Broadly Representative Antigen Sequences and Method for Selection

(76) Inventors: Adam C. Finnefrock, Berwyn, PA (US); Danilo R. Casimiro, Harleysville, PA (US); Jon H. Condra, Doylestown, PA (US); John W. Shiver, Doylestown, PA (US); Andrew J. Bett, Lansdale, PA (US)

Correspondence Address: MERCK, P O BOX 2000, RAHWAY, NJ, 07065-0907, US

(21) Appl. No.: 12/593,962

(22) PCT Filed: Mar. 26, 2008
(86) PCT No.: PCT/US08/03947

§ 371 Date: Sep. 30, 2009

Related U.S. Application Data
(60) Provisional application No. 60/921,020, filed on Mar. 30, 2007.

(51) Int. Cl. A61K 39/21; A61K 39/00; C12N 7/01; C12N 5/00; C12N 15/63; C07K 7/06; C07K 7/08; C07K 14/005; C07H 21/04

(52) U.S. Cl. 424/188.1; 424/185.1; 424/208.1; 435/235.1; 435/325; 435/320.1; 530/324; 530/325; 530/326; 530/327; 530/328; 530/329; 536/23.72

(57) ABSTRACT

A novel method for generating vaccine sequences is disclosed herein that preserves contiguous epitope length stretches of amino acids or nucleotides from an input pool of sequences. The method generates continuous, stepwise epitope consensus that together provides for a single globally optimized sequence. The end sequences are designed to maximize overlap between any potential epitope length sequence extract from a natural antigen sequence. The disclosed method, thus, allows one to maximize the number of potential natural epitopes that are mimicked in a resultant vaccine sequence. Various representative HIV vaccine sequences have been generated and are disclosed herein.

### CROSS-REFERENCE TO RELATED APPLICATIONS

This application claims the benefit of U.S. Provisional Application No. 60/921,020, filed Mar. 30, 2007, which is herein incorporated by reference in its entirety.

### FIELD OF THE INVENTION

The present invention relates to the field of vaccines, and particularly to vaccines that elicit a cell-mediated immune response. The present invention, furthermore, relates to a field of bioinformatics, more specifically immunoinformatics by providing a method for the generation of vaccine antigens that are capable, through their composition, of eliciting a broadly reactive immune response that is capable of recognizing multiple pathogens or cancer antigens.

### BACKGROUND OF THE INVENTION

Antigen selection is critical to the design of effective vaccines for infectious diseases. Optimally, the antigen selected is capable of inducing a broad immune response that is either simultaneously directed against multiple epitopes and/or capable of recognizing multiple viral subtypes. Eliciting this more “comprehensive” immune response as described is of particular import when considering pathogens that possess the innate ability to mutate and evade the host immune response including, but not limited to, Hepatitis C virus (HCV), Hepatitis B Virus (HBV), or Human Immunodeficiency Virus (HIV). Faced with these types of pathogens, the T-lymphocyte cellular-mediated immune (“CMI”) response forms a critical component of the immune response. T cell-mediated immune responses require the activation of cytotoxic (CD8+) and helper (CD4+) T lymphocytes. T lymphocytes (CTL) and their T-cell receptors (TCR) recognize small peptides presented by major histocompatibility complex (MHC) class I (in the case of CD8+) and class II (in the case of CD4+) molecules on the cell surface; Bjorkman P J., 1997 Cell 89:167-170; Garcia et al., 1996 Science 274:209-219. The peptides are derived from intracellular antigens via the endogenous antigen processing and presentation pathway; Germain R N., 1994 Cell 76:287-299; Pamer et al., 1998 Annu Rev Immunol 16:323-358. Peptides for human CD8+ epitopes range from 7 to 14 amino acids, and typically are 9-10 amino acids in length. Peptides for CD4+ epitopes have been reported as short as 9 amino acids in length, and as long as 20 amino acids in length, with typical lengths of approximately 15-16 amino acids; HIV Molecular Immunology, 2005, Eds. B T M Korber et al., Publisher: Los Alamos National Laboratory, Theoretical Biology and Biophysics, Los Alamos, New Mexico. LA-UR 06-0036. TCR recognition of the peptide-MHC class II molecule complexes on the cell surface trigger the production of a number of cytokines. These cytokines help to fully activate the CD8+-mediated response. TCR recognition of the peptide-MHC class I molecule complexes on the cell surface triggers the cytolytic activity of CTL, resulting in the death of cells presenting the peptide-MHC class I complexes; Kagi et al., 1994 Science 265:528-530. Partly because of this cytotoxic function, CTL responses have been implicated as playing an important role in control of viral infection; Kagi & Hengartner, 1996 Curr Opin Immunol 8:472-477; Letvin N L, 1998 Science 280:1875-1880; Yang et al., 1996 J Virol 70:5799-5806.

CMI responses have been particularly implicated in the control of human immunodeficiency virus (HIV) infection. The appearance of vigorous CTL responses in HIV-1 or simian immunodeficiency virus (SIV)-infected subjects has been found to be temporally associated with the control of primary viral infection; Borrow et al., 1994 J Virol 68:6103-6110; Koup et al., 1994 J Virol 68:4650-4655; Kuroda et al., 1999 J Immunol 162:5127-5133. Additionally, studies showed that vigorous CTL responses in HIV-infected individuals exerts strong selective pressure on the virus in the hosts to evolve escape mutants; Borrow et al., 1997 Nat Med 3:205-211; McMichael et al., 1997 Annu Rev Immunol 15:271-296. Strong T-cell immunity has been associated with effective control of viremia and prolonged prevention of disease progression in HIV-infected patients; Harrer et al., 1996 J Immunol 156:2616-2623; Haynes et al., 1996 Science 271:324-328; Musey et al., 1997 N Engl J Med 337:1267-1274; Pontesilli et al., 1998 J Infect Dis 178:1008-1018. The frequency of CTL precursors (CTLp), determined by limiting dilution assay and by CTL epitope-specific tetramer staining of T cells, has been shown to be inversely correlated with virus load in SIV-infected rhesus macaques and HIV-infected human subjects, respectively; Gallimore et al., 1995 Nat Med 1:1167-1173; Ogg et al., 1998 Science 279:2103-2106. Lastly, in an SIV-infected rhesus macaque model, it has been shown in two independent studies that rhesus monkeys failed to control viral infection when their CD8⁺ T-cell population was depleted by administration of anti-CD8 monoclonal antibodies prior to acute infection or during chronic infection; Schmitz et al., 1999 Science 283:857-860; Jin et al., 1999 J Exp Med 189:991-998.

To date, sequences for vaccine antigens have typically been derived from isolates (e.g., viral sequences found in a patient) or from consensus sequences of viral isolates. The former relies on one particular antigen to elicit a broadly reactive immune response capable of cross-type recognition. The latter, consensus-type sequences, suffer from several problems. For one, they fail to weight contributions from different patients appropriately. Subjects who contribute more viral subtypes to the dataset may contribute disproportionately. While this may be partially mitigated by taking one sequence per patient, the resultant analysis then fails to take advantage of all available viral sequence data. A true consensus, as generally and previously defined, furthermore involves the aligning of multiple sequences and then selecting the most frequent amino acid (or nucleotide) at each position. This type of strategy has the undesirable attribute of generating artificial junctions (i.e., junctions not found in any of the input sequences utilized). Such artificial junctions are a problem for vaccines in general but in particular for T-cell based vaccines because they disrupt natural T-cell epitopes that are cleaved from fragments of vaccine sequences. In the presence of artificial junctions, T-cell responses could be directed to epitopes that are not present in the biologic target (defined as pathogen or self-antigen, e.g., cancer epitopes). Additionally, real epitopes that are present in the biologic target may not be included in the vaccine. The multiple alignments required as immediate steps to deriving a consensus are, furthermore, tedious and highly computer processing unit (CPU)-intensive. Each sequence pair must be aligned, so the number of operations scales as N²; with N being the length of the epitope of interest. Additionally, multiple alignments often contain errors due to the fact that they are only locally optimized (comparing the exact section of interest), not globally optimized. Subjective review is required which is very painstaking where many input sequences are considered. Resolution of difficult alignments may also be ambiguous. Two experts may legitimately generate different final alignments and, ultimately, different consensus sequences whose quality is difficult to assess.

The challenge of developing effective vaccines is in general complicated by sequence diversity. HIV exemplifies a particularly difficult instance. HIV diversity results from several factors, including high viral replication and error rates, prolonged courses of infection, viral adaptation to immune and drug pressures, and the deposition of infecting virus and its descendants into long-lived proviral reservoirs from which they may ultimately re-emerge. Besides evading the humoral and cell-mediated immune response in a single host, this leads to an astonishing diversity in the HIV virus within a local population; McCutchan et al., 2000 AIDS Res Hum Retroviruses 16:801-805, and globally; McCutchan et al., 2006 J Med Virol 78:S7-S12. In the face of geographic and social isolation of infected individuals, HIV-1 replication has given rise to multiple independently evolving viral lineages. To date, 15 major HIV-1 clades and numerous inter-clade circulating recombinant forms have been recognized worldwide; Leitner, et al., HIV Sequence Compendium 2005, Theoretical Biology and Biophysics Group, Los Alamos National Laboratory, Los Alamos, N. Mex.

To address the complexity for HIV and other sets of diverse natural antigenic sequences, several approaches have been attempted. One is to select a sequence based upon a single antigen sequence that is typical of many other sequences, or close to the global or clade-specific consensus. An example of this is the HIV gag CAM-1 sequence, which is similar to many HIV clade B sequences. Other approaches include consensus or putative ancestral approaches; Korber B, 2001 Br Med Bull 58:19-42; Gaschen, et al., 2002 Science 296:2354-2360; International Publication No. WO2005/028625 and center of tree modifications thereof; Nickle et al., 2003 Science 299:1515-1518; Mullins et al., 2004 Expert Rev Vaccines 3:S151-S159 have been proposed as potential immunogens in order to minimize overall genetic distances between vaccine and target viruses. The assumption beneath the ancestral and center-of-tree approaches is that a hypothetical ancestral sequence, although not necessarily present in present-day antigen sequences, is representative of the entire present-day set of antigen sequences. However, with the consensus and ancestral approaches, the resulting sequences are artificial composites of multiple natural viral sequences that do not necessarily represent existing natural antigenic sequences and more problematically, could present artificial T cell epitopes if used as vaccine antigens.

A method of designing vaccine immunogens is described in Fischer, et al., 2007 Nature Medicine 13:100-106. The Fischer et al. method incorporates a stochastic approach (a random sampling) within a sequence space, rather than a deterministic approach where, for a given input data set, the same resultant optimal sequences are returned. Additionally, the Fischer et al. method creates mosaic sequences, where continuity between the resultant sequence and broad regions of the input antigen sequences is not necessarily assured. In particular, continuity is not assured across any given set of N amino acids. The method, furthermore, employs a genetic algorithm.

In published U.S. application, US 2006/0178861, a machine-learning algorithm is described to create vaccine cocktails to maximize a general function across sequence fragments (“patches”). One general function might have as its goal to maximize epitope coverage. In paragraph [0029] of the aforementioned application, a mapping is described between a set of fragments and sequence indices and a set of patches in a resulting sequence (“epitome”). There is no criteria to guarantee or optimize continuity throughout the resultant sequences through every possible N-mer sequence, with no artificial junctions (junctions not found in one of the natural antigen sequences). The published method, furthermore, does not teach the use of every possible N-mer and every sequence in the input data set. The published method also involves a machine-learning algorithm, an arbitrary cost function, and an energy function that follows a Boltzmann-like (statistical mechanics equilibrium) distribution of states.

The disclosed method and sequences improve upon the art by offering methods and resultant sequences that address some of the problems noted with the traditional consensus sequences. As a result, sequences derived hereby are better able to elicit a more broadly reactive immune response in treated subjects.

### SUMMARY OF THE INVENTION

The present invention relates to a novel method for generating vaccine sequences. The method preserves contiguous epitope length stretches of amino acids or nucleotides from an input pool of sequences and eliminates the need to generate intermediate multiple-sequence alignments. The method involves the generation of a continuous, stepwise epitope consensus, which in its entirety provides for a single globally optimized sequence. The goal of designing the antigen sequence in this manner is to maximize overlap between any and all potential epitope length sequences present. The disclosed method, thus, allows one to maximize the number of potential natural epitopes mimicked in the vaccine antigen sequence.

To illustrate, take the following four sequences:

| | |
|---|---|
| ACDEFGHIKLMN | SEQ ID NO: 48 |
| ACDEHGHIKLMN | SEQ ID NO: 49 |
| ACDEWNHIKLMN | SEQ ID NO: 50 |
| ACDEWLHIKLMN | SEQ ID NO: 51 |

A true consensus, as generally and previously defined, involves the aligning of multiple sequences and then selecting the most frequent amino acid (or nucleotide) at each position. A true consensus of the foregoing sequences would be: ACDEWGHIKLMN; SEQ ID NO: 52. This consensus sequence has the undesirable attribute of an artificial junction (i.e., a junction not found in any input sequence). “WG” is present in the derived consensus but is not present in any of the input sequences. This artificial junction is a problem for vaccines in general but in particular for T-cell based vaccines because it disrupts natural T-cell epitopes that are cleaved from fragments of vaccine sequences. In the presence of artificial junctions, T-cell responses could be directed to epitopes that are not present in the biologic target (defined as pathogen or self-antigen, e.g., cancer epitopes). Additionally, real epitopes that are present in the biologic target may not be included in the vaccine.

In the methods of the present invention, a single globally optimized solution is developed that, by design, is unable to generate artificial junctions because each overlapping amino acid epitope-length section or fragment of the resultant sequence is guaranteed to be from a natural input sequence or natural antigen sequence as referred to herein.

The disclosed methods, furthermore, incorporate a patient-weighted consensus. All sequence information is considered in the method, but every patient contributes equally to the consensus.

The disclosed methods, therefore, relate in specific embodiments to a method for generating consensus sequences of use in vaccination, which comprises:

(a) compiling a population of two or more sequences from a target antigen of interest (particular natural antigen sequence of interest);

(b) deriving substantially all possible overlapping successive sequence fragments (“N-mers”) for the sequences in the population; said N-mers characterized as being of a length (“N”) which comprises at least one epitope of interest; wherein “N” is any number from about 7 to about 30; and

(c) adding successive amino acids, first to an initial N-mer (a stretch of N amino acids that begin a sequence in (a)) by identifying a fragment(s) overlapping the preceding N-mer by N−1 amino acids and adding the last amino acid of the fragment(s); and repeating this procedure until ending with the final amino acid of a terminal N-mer (a stretch of N amino acids that end a sequence in (a));

wherein the consensus sequences have at least 90% of every successive N-mer sequence present in a natural antigen sequence. In specific embodiments, the consensus sequences comprise N-mer sequence from at least three different natural antigen sequences and, in additional specific embodiments, from at least six, and from at least ten different natural antigen sequences, in order of increasing preference.

The two or more sequences compiled in step (a) are unique sequences for a particular natural antigen sequence of a pathogenic agent or target antigen which are derived directly or indirectly from a mammalian sample.

The disclosed methods, furthermore, relate in specific embodiments to a method for generating and comparing or ranking consensus sequences of use in vaccination, which comprises:

(a) compiling a population of two or more sequences from a target antigen of interest (particular natural antigen sequence of interest);

(b) deriving substantially all possible overlapping successive sequence fragments (“N-mers”) for the sequences of the population; said N-mers characterized as being of a length (“N”) which comprises at least one epitope of interest; wherein “N” is any number from about 7 to about 30;

(c) individually assigning each fragment a weight proportional to the number of natural antigen sequences provided per patient or subject (“input sequences”) (in specific embodiments, the weight assigned may be equal to 1/M; “M” being the number of sequences provided per patient or subject); said input sequences being unique sequences for a particular natural antigen sequence of a pathogenic agent or target antigen which are derived directly or indirectly from any one mammalian sample.

(d) optionally, adjusting the weights of (c) according to the prevalence of each sequence within a particular clade, subtype or geographic region or according to the pathogenicity or oncogenicity of each sequence as determined, for example, through epidemiological estimation. This may be carried out, for example, in specific embodiments by multiplying each fragment's weight in (c) by another weighting factor that is a function of clade, geographic region, pathogenicity, or oncogenicity, particularly where the factor is proportional to the prevalence of the sequence in a clade or geographic region or epidemiological estimation of the pathogenicity or oncogenicity;

(e) providing a score to each fragment based on the number of times said fragment appears in the input sequences and the weight in (c) and/or (d);

(f) adding successive amino acids, first to an initial N-mer (a stretch of N amino acids that begin a sequence in (a)) by identifying a fragment(s) overlapping the preceding N-mer by N−1 amino acids and adding the last amino acid of the fragment(s); and repeating this procedure until ending with the final amino acid of a terminal N-mer (a stretch of N amino acids that end a sequence in (a));

(g) calculating the cumulative total score of the successive sequence fragments of the sequences produced in step (f); and

(h) comparing and/or ranking the consensus sequences based on total score;

wherein the consensus sequences have at least 90% of every successive N-mer sequence present in a natural antigen sequence. In specific embodiments, the consensus sequences comprise N-mer sequence from at least three different natural antigen sequences and, in additional specific embodiments, from at least six, and from at least ten different natural antigen sequences, in order of increasing preference.

The two or more sequences compiled in step (a) are unique sequences for a particular natural antigen sequence of a pathogenic agent or target antigen which are derived directly or indirectly from a mammalian sample.

In preferred embodiments, the consensus sequences have at least 90%, 95%, 96%, 97%, 98%, 99% and 100% of every successive N-mer sequence present in a natural antigen sequence, in order of increasing preference. Specific embodiments of the present invention relate to antigen sequences wherein every 8-, 9-, 15-, 16- or 30-mer extract of the consensus sequence is present in a natural antigen sequence. In specific embodiments, the resultant consensus sequences are, furthermore, not found in a natural antigen sequence.

Through the described methods, overlapping successive N-mer sequence fragments are combined to form a single continuous sequence such that any N-mer extract of the sequence can be traced to a natural antigen sequence. The N-mers that comprise the sequence may be chosen to maximize the total overlap with a global set of target antigen sequences. The sequences are, additionally, weighted such that all patients forming the input pool are given equal weight, and the isolates, subtypes, samples or clades (as the case may be) forming the input pool are represented according to their estimated global prevalence, irrespective of their arbitrary frequency in sequence databases.

A key property is that for practically the entire vaccine sequence (>90% and, in order of increasing preference, 95%, 96%, 97%, 98%, 99% and 100% of the vaccine sequence), any continuous stretch of 30 (or fewer, depending on the chosen N-mer size) amino acids can be found in an actual viral isolate, pathogen or cancer sample. This is in contrast to other putative vaccine sequences where specific fragments are combined with synthetic linkers and this property termed N-mer continuity is not maintained. Given the well-appreciated complexity of the epitope processing and presentation, it is impossible to predict with certainty which peptides will be cleaved from a polypeptide sequence. This is particularly true for HLA-types which have been less studied such as are found in most parts of the world. As such, it is highly desirable for an immune response that is directed against the desired vaccine that every potential peptide (>90% and, in order of increasing preference, 95%, 96%, 97%, 98%, 99% and 100%) that is excised and presented on the cell surface be representative of the virus or disease protein against which an immune response is designed to be elicited through the vaccine. Artificial peptide fragments that do not correspond to the virus or disease protein have the potential to misdirect the dominant immune response towards irrelevant epitopes that would have no capability to protect.

The consensus sequences may be derived from any antigen of interest provided the antigen is capable of inducing a cell-mediated immune response. Such consensus sequences include but are not limited to, sequences derived from any biological entity that causes pathological symptoms when present in a mammalian host. The biological entity may be, without limitation, an infectious agent (e.g., a virus, a prion, a bacterium, a yeast or other fungus, a mycoplasma, or a eukaryotic parasite such as a protozoan parasite, a nematode parasite, or a trematode parasite) or a tumor antigen (e.g., a lung cancer or a breast cancer antigen).

In specific embodiments, the N-mer can be any amino acid sequence of any length that encompasses standard epitopes. In specific embodiments, this ranges from about 7 amino acids to about 30 amino acids. The number of amino acids for CD8+ (CTL) epitopes may range from 7 to 14 amino acids, with typical ranges being from 9 to 10 amino acids. The number of amino acids for CD4+ (helper) epitopes has been reported to range from 9 amino acids in length to as long as 20 amino acids in length, with typical ranges from 15-16 amino acids. The present invention encompasses N-mers of all these ranges. The specific N-mer chosen will depend on the epitope range being sought. In particular embodiments, the N-mer is selected from the group consisting of: an 8-mer, a 9-mer, a 15-mer, a 16-mer and a 30-mer.

The present invention relates as well to antigen sequences wherein at least 90% (and, in specific embodiments, at least 95%, 96%, 97%, 98%, 99% and 100%, in order of increasing preference) of every successive N-mer sequence is present in a natural antigen sequence. Specific embodiments of the present invention relate to antigen sequences wherein every 8-, 9-, 15-, 16- or 30-mer extract of the consensus sequence is present in a natural antigen sequence. Specific embodiments also provide for consensus antigen sequences as described wherein the resultant consensus sequence is not found in a natural antigen sequence.

The present invention, furthermore, relates to antigen sequences which comprise N-mer sequences from at least three different natural antigen sequences and, in specific embodiments, at least six, and at least ten different natural antigen sequences in preferred embodiments, in order of increasing preference.

The present invention, additionally, relates to a series of HIV vaccine sequences that are characterized as having successive N-mer fragments from HIV-1 viral isolates found in infected humans.

### TERMS

Unless defined otherwise, technical and scientific terms used herein have the meanings commonly understood by one of ordinary skill in the art to which the present invention pertains. One skilled in the art will recognize other methods and materials similar or equivalent to those described herein, which can be used in the practice of the present teachings. It is to be understood, that the teachings presented herein are not intended to limit the methodology or processes described herein. For purposes of the present invention, the following terms are defined below.

As used herein, the terms “8-mer”, “9-mer”, “15-mer”, “16-mer”, “30-mer” and “N-mer” refer to a linear sequence of eight, nine, fifteen, sixteen, thirty or N amino acids, respectively, that occur in a target antigen.

As used herein, the term “antigen” refers to any biologic or macromolecular substance that can be recognized by a T-cell or an antibody molecule.

As used herein, the terms “major histocompatibility complex (MHC)” and “human leukocyte antigen (HLA)” are used interchangeably to refer to a locus of genes that encode proteins, or the proteins themselves, which present a vast variety of peptides onto the cell surface for specific recognition by a T-cell receptor.

A subclass of MHC, called Class I MHC molecules, present peptides to CD8 T-cells.

As used herein, an “immunogen” refers to a specific antigen capable of inducing or stimulating an immune response. Not all antigens are immunogenic.

As used herein, an “epitope” refers to a peptide comprising an amino acid sequence that is capable of stimulating an immune response. MHC class I epitopes may be used in compositions (e.g., vaccines) for stimulating an immune response directed to the target antigen.

A “target antigen” as used herein refers to an antigen of interest to which an immune response may be directed or stimulated, including but not limited to pathogenic (e.g., derived from a pathogenic agent) and tumor antigens (for purposes of exemplification and not limitation, a lung cancer or a breast cancer antigen).

As used herein, a “pathogenic agent” is a biological entity that causes pathological symptoms when present in a mammalian host. Thus a pathogenic agent can be, without limitation, an infectious agent (e.g., a virus, a prion, a bacterium, a yeast or other fungus, a mycoplasma, or a eukaryotic parasite such as a protozoan parasite, a nematode parasite, or a trematode parasite).

As used herein, a “natural antigen sequence” is a sequence for a pathogenic agent or target antigen which is derived directly or indirectly from a mammalian sample. The natural antigen sequence may be an actual viral isolate, pathogen or cancer sample. Actual derivation from a natural sequence avoids the artificial junctions found in previous consensus sequences. Natural antigen sequences may, in specific embodiments, be found, for example, in databases of patient isolates such as the Los Alamos database.

As used herein, the term “vaccine” is used to refer to those immunogenic compositions that are capable of eliciting prophylactic and/or therapeutic responses that prevent, cure, or ameliorate disease.

“Isolated” as used herein describes a property as it pertains to the nucleic acid, protein or other that makes it different from that found in nature. The difference may be, for example, that it is of a different purity than that found in nature, or that it is in a different structure or forms part of a different structure than that found in nature. An example of a nucleic acid sequence not found in nature is that substantially free of other cellular material.

### BRIEF DESCRIPTION OF THE DRAWINGS

FIG. 1 illustrates the global population-weighted scores for the supplemented Gag Cam1 and Nef JRFL as compared to the unsupplemented sequences. The sequences are compared by counting the number of 9-mer amino acid fragments that are found exactly in natural antigen sequences (weighted according to their estimated global prevalence), normalized to the total number of 9-mers in the natural antigen sequence. For every pair of vaccine/target sequences, the set of all successive 9-mers (aa1-9, aa2-10 . . . ) that can be taken from the vaccine sequence is compared with the set of all successive 9-mers (aa1-9, aa2-10 . . . ) from the target sequence. Each 9mer in the first set is compared against every 9mer in the second set, and the closest match is selected. The number of responses/matches between the vaccine and target sets are summed and normalized by the number of 9mers in the target set. Results across all targets are then weighted by the prevalence of their clade of origin and summed to yield a single final number as shown by the bar height in FIG. 1. The algorithm that calculates these scores may be practiced by the skilled artisan using the methods and materials described under Computer Hardware and Software below following the teachings herein. For this scoring algorithm, it is envisioned that the artisan would choose to implement the comparisons between each vaccine and target sequence in an efficient compiled language such as C or C++ or suitable alternative machine language.

FIGS. 2A-F illustrate an alignment of gag N16.1 (SEQ ID NO: 1) with a set of HIV-1 viral isolates (SEQ ID NOs: 5-9, respectively). Each 16-mer amino acid fragment of gag N16.1 can be found in one or more of the isolates.

FIGS. 3A-O illustrate an alignment of gag N16.2 (SEQ ID NO: 2) with a set of HIV-1 viral isolates (SEQ ID NOs: 10-23, respectively). Each 16-mer amino acid fragment of gag N16.2 can be found in one or more of the isolates.

FIGS. 4A-G illustrate an alignment of nef.N16.1 (SEQ ID NO: 3) with a set of HIV-1 viral isolates (SEQ ID NOs: 24-29, respectively). Each 16-mer amino acid fragment of nef N16.1 can be found in one or more of the isolates.

FIGS. 5A-J illustrate an alignment of nef.N16.2 (SEQ ID NO: 4) with a set of HIV-1 viral isolates (SEQ ID NOs: 30-38, respectively). Each 16-mer amino acid fragment of nef N16.2 can be found in one or more of the isolates.

FIG. 6 illustrates the MRKAd5GGNN adenoviral vector.

FIGS. 7A-B illustrate the construction of adenovirus vector MRKAd5GGNN.

FIG. 8 illustrates the MRKAd5GNGN adenoviral vector.

FIGS. 9A-B illustrate the construction of adenovirus vector MRKAd5GNGN.

FIG. 10 illustrates the MRKAd6GGNN adenoviral vector.

FIGS. 11A-B illustrate the construction of adenovirus vector MRKAd6GGNN.

FIG. 12 illustrates the MRKAd6GNGN adenoviral vector.

FIGS. 13A-B illustrate the construction of adenovirus vector MRKAd6GNGN.

FIG. 14 illustrates the MRKAd5GNNN adenoviral vector.

FIGS. 15A-B illustrate the construction of adenovirus vector MRKAd5GNNN.

FIG. 16 illustrates the MRKAd6GNNN adenoviral vector.

FIGS. 17A-B illustrate the construction of adenovirus vector MRKAd6GNNN.

FIG. 18 illustrates a Western blot for the detection of the GGNN and GNGN fusion proteins. The lanes are represented as follows: Lanes 1 & 8: Prestained Marker; Lane 2: Ad5gagpolnef; Lane 3: Ad5GGNN; Lane 4: Ad5GNGN; Lanes 5 & 12: Ad5SEAP; Lanes 6 & 13: Uninfected cells; Lanes 7 & 14: Affinity Magic Mark XP; Lane 9: Ad6gagpolnef; Lane 10: Ad6GGNN; and Lane 11: Ad6GNGN. The expected sizes were Gagpolnef: 176 kDa; Gaggagnefnef: 157 kDa; and Gagnefgagnef: 157 kDa.

FIG. 19 illustrates a Western blot for the detection of the GNNN fusion proteins. The lanes are represented as follows: Lane 1: Affinity Magic Mark XP; Lane 2: Uninfected cells; Lane 3: Ad6GNNN; Lane 4: Ad5GNNN; Lane 5: Ad6gagpolnef; Lane 6: Ad5gagpolnef; and Lane 7; Prestained Marker. The expected sizes were Gagpolnef: 176 kDa; and Gagnefnefnef: 126 kDa.

FIG. 20 illustrates the geometric means of ELISA endpoint titers to Gag and Nef proteins for mice immunized with vaccine constructs labeled on the X-axis.

FIGS. 21A-C illustrate the antibody levels in units/ml for Gag (a), Pol (b), and Nef (c) antigens, respectively, as a function of time of sampling in weeks post-injection.

### DETAILED DESCRIPTION OF THE INVENTION

The present invention relates to a novel method for generating consensus sequences of use in vaccination that preserves contiguous stretches of amino acids or nucleotides of epitope length from an input pool of sequences. Use of the method results in a single globally optimized sequence wherein overlap between the various overlapping possible epitope sequences is maximized.

The method comprises, first, compiling (gathering) a population of two or more sequences from a target antigen of interest. The two or more sequences compiled in step (a) are unique sequences for a particular natural antigen sequence of a pathogenic agent or target antigen which are derived directly or indirectly from a mammalian sample. Next, all successive sequence fragments of epitope length or a length which comprises an epitope of interest are derived from the population. “Successive sequence fragments” refers to every possible fragment of epitope length (or alternative encompassing length) starting from the beginning to the ending of the sequence. In other words, in the sequence ACDEFGHIKLMNRST (SEQ ID NO: 53) where a 9-mer epitope length is contemplated, the following would formulate the successive sequence fragments:

| | |
|---|---|
| ACDEFGHIK; | (SEQ ID NO 54) |
| CDEFGHIKL; | (SEQ ID NO: 55) |
| DEFGHIKLM; | (SEQ ID NO; 56) |
| EFGHIKLMN; | (SEQ ID NO; 57) |
| FGHIKLMNR; | (SEQ ID NO: 58) |
| GHIKLMNRS; | (SEQ ID NO: 59) |
| and | |
| HIKLMNRST. | (SEQ ID NO: 60) |

Where various sequences are used, the corresponding successive fragments would be analyzed alongside each other.

Use of the term epitope length is used in reference to the number of amino acids typically present in an epitope recognized by the immune system for the particular antigen of interest. The concept of an epitope is readily understood by the person of ordinary skill in the art. Human CD8+ epitopes generally range from 7 to 14 amino acids, with typical ranges being from 9 to 10 amino acids. The number of amino acids for CD4+ (helper) epitopes has been reported to range from 9 amino acids in length to as long as 20 amino acids in length, with typical ranges from 15-16 amino acids. It is well established that CD8+ cytotoxic T lymphocytes (“CTL”) play a crucial role in the eradication of infectious diseases by the mammalian immune system. It is, furthermore, well established that CD4+ assist the immune response in recognizing foreign antigen through the release of cytokine.

“N” as referred to herein may be any number of amino acids which comprises, or is considered to be representative, of the epitope/antigen being studied. In specific embodiments, the fragment length (N) is any number from about 7 to about 30. In more specific embodiments, the method is carried out employing an N of 8, 9, 15, 16 or 30.

Following generation of the successive sequence fragments, various successive sequence fragments are, preferably, assigned a weight of 1/M, wherein “M” is the number of sequences provided per 1 patient or subject. Inputting and evaluating subject viral sequence data in this manner forms an additional aspect of the present invention. The data may be maintained in a global list of “N-mers” (the term used hereafter to refer to a sequence encompassing a fragment of epitope length) and scored by frequency of occurrence, those with greater prevalence being scored higher. It is, moreover, preferable to store the initial N-mers, the interior N-mers (in order) and the terminal N-mers from each sequence in separate lists. Thus, for instance, in the sequence ACDEFGHIKLMNRST (SEQ ID NO: 53) where N=9, the following could form the lists:

| | |
|---|---|
| Initial N-mer | |
| ACDEFGHIK; | (SEQ ID NO 54) |
| Interior N-mers | |
| CDEFGHIKL; | (SEQ ID NO: 55) |
| DEFGHIKLM; | (SEQ ID NO; 56) |
| EFGHIKLMN; | (SEQ ID NO; 57) |
| FGHIKLMNR; | (SEQ ID NO: 58) |
| GHIKLMNRS; | (SEQ ID NO: 59) |
| Terminal N-Mer | |
| HIKLMNRST | (SEQ ID NO: 60) |

The initial N-mer(s) is used to nucleate (or start) a separate thread of amino acids. The sequence is gradually expanded by evaluating all N-mers from the population of successive N-mers that overlap by N−1 amino acids; “N” being the length of the epitope of interest. Where multiple overlapping N-mer candidates exist, the thread is copied to encompass all possibilities.

In the instance where there is not an overlapping subsequence (N−1)-mer sequence, the thread should be removed from consideration. In those situations where a terminal N-mer is reached, the thread is ended.

When all threads are complete (either by reaching a terminal N-mer or for which a terminal N-mer can not be found), the cumulative total score of every successive overlapping N-mer populating the thread may be calculated. Where an N-mer is present more than once in the thread, it preferably contributes to the total score only once. Equally, in the instance of multi-component vaccines, “redundant” N-mers (those present in more than 1 component), in preferred embodiments, are given a score of zero and only the original one would contribute to the total score.

The following methods, all of which are encompassed as specific embodiments herein, may be employed for ranking the sequence threads:

(1) Rank according to best overall score (“unconstrained”). This method matches the most N-mer segments from the input set. This method, therefore, tends to pick up insertions found in some but not all clones, and tends towards longer sequences.

(2) Rank according to best score per sequence length (“length-normalized”). This method is biased against insertions not found in many clones, and tends to pick up short, highly conserved regions.

(3) Rank by best score per sequence length (length-normalized), but require the first and last N-mer to match those from the unconstrained consensus (“constrained”). Constrained N-mer consensuses are biased against insertions not found in many clones but prevent partial sequences and are balanced between insertions and deletions. The total score is determined by the amount of matching N-mers divided by the number of N-mers.

Method (3) is particularly preferred for vaccine antigen selection.

The methods do not rely upon random numbers. Rather, the disclosed methods are deterministic, meaning that, for a given set of input, the method always produces the same optimal N-mer consensus sequence. The methods do not produce artificial junctions (junctions not found in one of the natural antigen sequences). The methods make use of every N-mer and every sequence in the input data set. The methods assure and maximize continuity across every N-mer sequence in the resultant N-mer consensus sequence. Also, the methods enable the skilled artisan to explicitly score and count multiple N-mers from the data set and incorporate these into the algorithm. The methods, furthermore, do not require or rely on a genetic algorithm or a machine-learning algorithm.

The disclosed methods, thus, relate in one aspect to a method for generating consensus sequences of use in vaccination, which comprises:

(a) compiling a population of two or more sequences from a target antigen of interest (a particular natural antigen sequence of interest);

(b) deriving substantially all possible overlapping successive sequence fragments (“N-mers”) for the sequences in the population; said N-mers characterized as being of a length (“N”) which comprises at least one epitope of interest; wherein “N” is any number from about 7 to about 30; and

(c) adding successive amino acids, first to an initial N-mer (a stretch of N amino acids that begin a sequence in (a)) by identifying a fragment(s) overlapping the preceding N-mer by N−1 amino acids and adding the last amino acid of the fragment(s), repeating this procedure until ending with the final amino acid of a terminal N-mer (a stretch of N amino acids that end a sequence in (a));

wherein the consensus sequences have at least 90% of every successive N-mer sequence present in a natural antigen sequence. In specific embodiments, the consensus sequences comprise N-mer sequence from at least three different natural antigen sequences and, in additional specific embodiments, from at least six, and from at least ten different natural antigen sequences, in order of increasing preference.

The two or more sequences compiled in step (a) are unique sequences for a particular natural antigen sequence of a pathogenic agent or target antigen which are derived directly or indirectly from a mammalian sample.

The disclosed methods, furthermore, relate in another aspect to a method for generating and ranking or comparing consensus sequences of use in vaccination, which comprises:

(a) compiling a population of two or more sequences from a target antigen of interest (a particular natural antigen sequence of interest);

(b) deriving substantially all possible overlapping successive sequence fragments (“N-mers”) for the sequences in the population; said N-mers characterized as being of a length (“N”) which comprises at least one epitope of interest; wherein “N” is any number from about 7 to about 30;

(c) individually assigning each fragment a weight proportional to the number of natural antigen sequences provided per patient or subject (“input sequences”) (in specific embodiments, the weight may be assigned as equal to 1/M; “M” being the number of sequences provided per patient or subject); said input sequences being unique sequences for a particular natural antigen sequence of a pathogenic agent or target antigen which are derived directly or indirectly from any one mammalian sample;

(d) optionally, adjusting the weights of (c) according to the prevalence of each sequence within a particular clade, subtype or geographic region or according to the pathogenicity or oncogenicity of each sequence as determined, for example, through epidemiological estimation. This may be carried out by, for example, in specific embodiments multiplying each fragment's weight in (c) by another weighting factor that is a function of clade, geographic region, pathogenicity, or oncogenicity, particularly where the factor is proportional to the prevalence of the sequence in a clade or geographic region or epidemiological estimation of the pathogenicity or oncogenicity;

(e) providing a score to each fragment based on the number of times said fragment appears in the input sequences and the weight of (c) and/or (d);

(f) adding successive amino acids, first to an initial N-mer (a stretch of N amino acids that begin a sequence in (a)) by identifying a fragment(s) overlapping the preceding N-mer by N−1 amino acids and adding the last amino acid of the fragment(s), repeating this procedure until ending with the final amino acid of a terminal N-mer (a stretch of N amino acids that end a sequence in (a));

(g) calculating the cumulative total score of the successive sequence fragments of the sequences produced in step (f); and

(h) ranking or comparing the consensus sequences based on total score;

wherein the consensus sequences have at least 90% of every successive N-mer sequence present in a natural antigen sequence. In specific embodiments, the consensus sequences comprise N-mer sequence from at least three different natural antigen sequences and, in additional specific embodiments, from at least six, and from at least ten different natural antigen sequences, in order of increasing preference.

The two or more sequences compiled in step (a) are unique sequences for a particular natural antigen sequence of a pathogenic agent or target antigen which are derived directly or indirectly from a mammalian sample.

In preferred embodiments, the consensus sequences have at least 90%, 95%, 96%, 97%, 98%, 99% and 100% of every successive N-mer sequence present in a natural antigen sequence, in order of increasing preference. Specific embodiments of the present invention relate to antigen sequences wherein every 8-, 9-, 15-, 16- or 30-mer extract of the consensus sequence is present in a natural antigen sequence. In specific embodiments, the resultant consensus sequences are, furthermore, not found in a natural antigen sequence.

The consensus sequences may be derived from any antigen of interest provided the antigen is capable of inducing a cell-mediated immune response. Such consensus sequences include but are not limited to, sequences derived from any biological entity that causes pathological symptoms when present in a mammalian host. The biological entity may be, without limitation, an infectious agent (e.g., a virus, a prion, a bacterium, a yeast or other fungus, a mycoplasma, or a eukaryotic parasite such as a protozoan parasite, a nematode parasite, or a trematode parasite) or a tumor antigen (e.g., a lung cancer or a breast cancer antigen).

In specific embodiments, the N-mer may be any amino acid sequence of a length that encompasses standard epitopes. In specific embodiments, this ranges from about 7 amino acids to about 30 amino acids. The number of amino acids for CD8+ (CTL) epitopes, in specific embodiments, may range from 7 to 14 amino acids, with typical ranges being from 9 to 10 amino acids. The number of amino acids for CD4+ (helper) epitopes, in specific embodiments, may range from 9 amino acids in length to as long as 20 amino acids in length, with typical ranges from 15-16 amino acids. The present invention encompasses N-mers falling within any of the above-specified ranges. The specific N-mer chosen will depend on the epitope range being sought. In particular embodiments, the N-mer is selected from the group consisting of: an 8-mer, a 9-mer, a 15-mer, a 16-mer and a 30-mer.

The methods of the present invention may be carried out through the use of the computer algorithm described herein.

### Computer Hardware and Software

The methods of the present invention may be carried out on a computer and may minimally involve: (a) inputting sequence data, and optionally, patient identification, population, and/or weighting data into an input device, e.g., through a keyboard, a diskette, CD-ROM, DVD-ROM, portable drive, network connection, or tape, and (b) determining, using a processor, one or more N-mer consensus sequences that maximize the matching score of N-mers within a suitably normalized and weighted set of sequences.

The invention described herein may be implemented with the use of computer hardware or software, or a combination of both. Generally speaking, various embodiments of the N-mer consensus algorithm described herein may be achieved with a computer program by providing instructions in a computer readable form. For example, the invention may be implemented by one or more computer programs executing on one or more programmable computers, each containing a processor and at least one input device. The computers will preferably also contain a data storage system (including volatile and non-volatile memory and/or storage elements) and at least one output device.

Program code is applied to input data to perform the functions described above and generate output information. The output information is applied to one or more output devices in a known fashion. The computer can be, for example, a personal computer, microcomputer, or workstation of conventional design. One of skill in the art will readily recognize that different types of computer language may be used to provide instructions in a computer readable format. For example, a suitable-computer program may be written in languages such as Matlab, C/C++, Python, FORTRAN, Perl, HTML, JAVA, UNIX, or LINUX shell command languages such as C shell or Korn shell scripts, and different dialects of the preceding languages. Each program is preferably implemented in a high level procedural or object oriented programming language to communicate with a computer system. However, the programs may be implemented in assembly or machine language, if desired. In any case, the language may be a compiled or interpreted language.

Each computer program is preferably stored on a storage media or device (e.g., ROM or magnetic diskette) readable by a general or special purpose programmable computer. The computer program serves to configure and operate the computer to perform the procedures described herein when the program is read by the computer. The method of the invention may also be implemented by means of a computer-readable storage medium, configured with a computer program, where the storage medium so configured causes a computer to operate in a specific and predefined manner to perform the functions described herein.

Different types of computers may be used to run a program implementing the algorithm described herein. For example, computer programs for carrying out the disclosed methods using the disclosed algorithm may be run on a computer having sufficient memory and processing capability. An example of a suitable computer is one having an Intel Pentium® (Intel Corp., Santa Clara, Calif.)-based processor of 200 MHz or greater, with 128 MB of main memory. Equivalent and superior computer systems are well known in the art. Faster processors will shorten the time to produce a result, while more memory permits a larger number of in-progress sequences to be held in memory at one time.

Standard operating systems may be employed for different types of computers. Examples of operating systems for an Intel Pentium®-based processor include the LINUX and variants thereof, and the MICROSOFT WINDOWS™ (Microsoft Corp., Redmond, Wash.) family, such as Windows Vista®, Windows NT®, Windows XP®, and Windows 2000; examples of operating systems for an Apple Macintosh® (Apple Inc., Cupertino, Calif.) computer include OS-X, UNIX and Linux operating systems; other computers Sun or SGI workstations running UNIX or LINUX related operating systems. Other computers and operating systems are well known in the art.

Examples are provided below to further illustrate different features of the present invention. The examples also illustrate useful methodology for practicing the invention. It is to be understood that these examples are not intended to limit the scope of the claimed invention.

The algorithms may be implemented in any fashion using one or more readily available modern computer programming language. The implementation and identification of such programming is well appreciated by the skilled artisan. In specific embodiments, these may be realized by programs that rely on ancillary software available to anyone without cost; many programs of which are extensively documented via the interne, downloadable hardcopy, or printed manuals in book form. Of particular use as ancillary software in specific embodiments is Open Source for which source code is available which can be compiled on a variety of hardware and software architectures. In specific embodiments, an HP xw8200 dual-Xeon processor workstation running Linux with 2 GB RAM and the programs detailed in Table 1 below are employed by the skilled artisan. The version numbers detailed below were current at the time of the practice of this invention, but it is anticipated that the artisan will use the most current stable release of each software package or language.

TABLE 1

| | VERSION | |
|---|---|---|
| NAME | (MAJOR) | DESCRIPTION |
| Python | 2.4 | Computer language |
| Numeric | 24 | Array toolkit for Python |
| Biopython | 1.41 | Bioinformatics toolkit for Python |
| Clustal W | 1.83 | Multiple sequence alignment |
| Gnu C | 3.4 | Computer language |

Any suitable materials and/or methods known to those of skill may be utilized to carry out the present invention; however, preferred materials and/or methods are described. It is believed that one skilled in the art may, based on the description herein, utilize the present invention to its fullest extent. The entire contents of all of the references (including literature references, issued patents, published patent applications, and co-pending patent applications) cited throughout this application are hereby expressly incorporated by reference.

It is important to note that the invention, however, is not reliant on any specific program. There are many programs available to the skilled artisan, any one or more of which can carry out the above methods. In fact, it is contemplated that the most efficient program or combination of programs available at the time of practice of the invention will be employed. The computing requirements are modest and any of a variety of approaches is sufficient to practice the invention. The ideas behind the methods are what is critical and what affect the outcome, not the means employed to arrive there. Methods described herein are purely illustrative.

Nucleic acids of use in, and derivable through, the methods of the present invention encode immunogenic proteins recognized by cell-mediated immune responses, more specifically by CD8+ and/or CD4+ cells. Preferred immunogenic proteins are those proteins which are capable of eliciting a protective and/or beneficial immune response in an individual.

As such, the present invention provides, in specific embodiments, compositions, recombinant protein sequences, encoding nucleic acid sequences, vectors, host cells, and methods of employing the foregoing which comprise, encode a protein which comprises, or utilize an amino acid sequence which comprises at least 90% and preferably, in order of increasing preference 95%, 96%, 97%, 98%, 99% and 100% of every continuous stretch of 30 (or fewer, depending on the chosen N-mer size) amino acids present or found in an actual viral isolate, pathogen or cancer sample. In specific embodiments, the selected N-mer size is an 8-, 9-, 15-, 16- or 30-mer. In specific embodiments, the amino acid sequence is, furthermore, derived from at least three different natural antigen sequences and, in specific embodiments, at least six, and at least ten different natural antigen sequences, in order of increasing preference. As the skilled artisan will no doubt appreciate, a greater number of sequences factored in or included in the dataset enhances the effectiveness of the consensus sequences for eliciting a broadly reactive immune response. This is because the expressed proteins, through the presentation of epitopes representative of various different natural strains or sequences, are capable of eliciting a more broadly cross-reactive immune response.

The present invention, furthermore, provides for compositions, recombinant protein sequences, encoding nucleic acid sequences, vectors, host cells, and methods of employing the foregoing which comprise, encode a protein which comprises, or utilize fragments of the disclosed consensus sequences. “Fragments” as defined herein refer to fragments of a consensus sequence (nucleotide or protein) which are capable of eliciting a significant cell-mediated immune response (as determined by various cellular assays available and widely appreciated by the skilled artisan; for purposes of exemplification and not limitation, for HIV antigens, this may be determined in an ELISpot assay by a result of, for example, >55 spots/10⁶ cells and ≧4× Mock). The sequence of the fragment or sequence comprising the fragment should hybridize under stringent conditions to the complement of at least one natural antigen sequence from which it was derived (directly or indirectly). Methods for hybridizing nucleic acids are well-known in the art; see, e.g., Ausubel, Current Protocols in Molecular Biology, John Wiley & Sons, N.Y., 6.3.1-6.3.6, 1989. For purposes of exemplification and not limitation, moderately stringent hybridization conditions may, in specific embodiments, use a prewashing solution containing 5× sodium chloride/sodium citrate (SSC), 0.5% w/v SDS, 1.0 mM EDTA (pH 8.0), hybridization buffer of about 50% v/v formamide, 6×SSC, and a hybridization temperature of 55° C. (or other similar hybridization solutions, such as one containing about 50% v/v formamide, with a hybridization temperature of 42° C.), and washing conditions of 60° C., in 0.5×SSC, 0.1% w/v SDS. For purposes of exemplification and not limitation, stringent hybridization conditions may, in specific embodiments, use the following conditions: 6×SSC at 45° C., followed by one or more washes in 0.1×SSC, 0.2% SDS at 68° C. One of skill in the art may, furthermore, manipulate the hybridization and/or washing conditions to increase or decrease the stringency of hybridization such that nucleic acids comprising nucleotide sequences that are at least 80, 85, 90, 95, 98, or 99% identical to each other typically remain hybridized to each other. The basic parameters affecting the choice of hybridization conditions and guidance for devising suitable conditions are set forth by Sambrook et al., Molecular Cloning: A Laboratory Manual, Cold Spring Harbor Laboratory Press, Cold Spring Harbor, N.Y., chapters 9 and 11, 1989 and Ausubel et al. (eds), Current Protocols in Molecular Biology, John Wiley & Sons, Inc., sections 2.10 and 6.3-6.4, 1995. Such parameters can be readily determined by those having ordinary skill in the art based on, for example, the length and/or base composition of the DNA.

The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-488; (61) amino acids 481-491; said amino acid numbers from SEQ ID NO: 1, SEQ ID NO: 67, SEQ ID NO: 75 or SEQ ID NO: 76. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-488; (61) amino acids 481-498; said amino acid numbers from SEQ ID NO: 2 or SEQ ID NO: 72. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-486; said amino acid numbers from SEQ ID NO: 64. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-479; said amino acid numbers from SEQ ID NO: 65. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-488; (61) amino acids 481-495; said amino acid numbers from SEQ ID NO: 66. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-488; (61) amino acids 481-499; said amino acid numbers from SEQ ID NO: 68. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-492; said amino acid numbers from SEQ ID NO: 69. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-488; (61) amino acids 481-500; said amino acid numbers from SEQ ID NO: 70. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-488; (61) amino acids 481-496; said amino acid numbers from SEQ ID NO: 71 or SEQ ID NO: 74. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-488; (61) amino acids 481-493; said amino acid numbers from SEQ ID NO: 73. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-206; said amino acid numbers from SEQ ID NO: 3, SEQ ID NO: 4, SEQ ID NO: 78, SEQ ID NO: 83, SEQ ID NO: 85, SEQ ID NO: 86, SEQ ID NO: 87, SEQ ID NO: 89, SEQ ID NO: 90, SEQ ID NO: 92, SEQ ID NO: 93, SEQ ID NO: 98, SEQ ID NO: 103, SEQ ID NO: 105, SEQ ID NO: 106; SEQ ID NO: 107; SEQ ID NO: 109 or SEQ ID NO: 110. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-173; said amino acid numbers from SEQ ID NO: 77, SEQ ID NO: 81, SEQ ID NO: 97 or SEQ ID NO: 101. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-198; said amino acid numbers from SEQ ID NO: 79 or SEQ ID NO: 99. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; said amino acid numbers from SEQ ID NO: 80 or SEQ ID NO: 100. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-207; said amino acid numbers from SEQ ID NO: 82, SEQ ID NO: 84, SEQ ID NO: 88, SEQ ID NO: 102, SEQ ID NO: 104 or SEQ ID NO: 108. The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136; (17) amino acids 129-144; (18) amino acids 137-152; (19) amino acids 145-160; (20) amino acids 153-168; (21) amino acids 161-176; (22) amino acids 169-184; (23) amino acids 177-192; (24) amino acids 185-200; (25) amino acids 193-208; (26) amino acids 201-216; (27) amino acids 209-224; (28) amino acids 217-232; (29) amino acids 225-240; (30) amino acids 233-248; (31) amino acids 241-256; (32) amino acids 249-264; (33) amino acids 257-272; (34) amino acids 265-280; (35) amino acids 273-288; (36) amino acids 281-296; (37) amino acids 289-304; (38) amino acids 297-312; (39) amino acids 305-320; (40) amino acids 313-328; (41) amino acids 321-336; (42) amino acids 329-344; (43) amino acids 337-352; (44) amino acids 345-360; (45) amino acids 353-368; (46) amino acids 361-376; (47) amino acids 369-384; (48) amino acids 377-392; (49) amino acids 385-400; (50) amino acids 393-408; (51) amino acids 401-416; (52) amino acids 409-424; (53) amino acids 417-432; (54) amino acids 425-440; (55) amino acids 433-448; (56) amino acids 441-456; (57) amino acids 449-464; (58) amino acids 457-472; (59) amino acids 465-480; (60) amino acids 473-488; (61) amino acids 481-496; (62) amino acids 489-504; (63) amino acids 497-512; (64) amino acids 505-520; (65) amino acids 513-528; (66) amino acids 521-536; (67) amino acids 529-544; (68) amino acids 537-552; (69) amino acids 545-560; (70) amino acids 553-568; (71) amino acids 561-576; (72) amino acids 569-584; (73) amino acids 577-592; (74) amino acids 585-600; (75) amino acids 593-608; (76) amino acids 601-616; (77) amino acids 609-624; (78) amino acids 617-632; (79) amino acids 625-640; (80) amino acids 633-648; (81) amino acids 641-656; (82) amino acids 649-664; (83) amino acids 657-672; (84) amino acids 665-680; (85) amino acids 673-688; (86) amino acids 681-696; (87) amino acids 689-704; (88) amino acids 697-712; (89) amino acids 705-720; (90) amino acids 713-728; (91) amino acids 721-736; (92) amino acids 729-744; (93) amino acids 737-752; (94) amino acids 745-760; (95) amino acids 753-768; (96) amino acids 761-776; (97) amino acids 769-784; (98) amino acids 777-792; (99) amino acids 785-800; (100) amino acids 793-808; (101) amino acids 801-816; (102) amino acids 809-824; (103) amino acids 817-832; (104) amino acids 825-840; (105) amino acids 833-848; (106) amino acids 841-850; said amino acid numbers from SEQ ID NO: 112.

“Fusions” as encompassed herein are any sequences (nucleic acid or protein) which comprise at least one of the consensus sequences disclosed herein fused to at least one other antigen consensus sequence or consensus sequence disclosed herein.

The present invention, furthermore, provides in specific embodiments compositions, recombinant protein sequences, encoding nucleic acid sequences, vectors, host cells, and methods of employing the foregoing which comprise, encode a protein which comprises, or utilize an amino acid sequence which comprises two or more sequences, at least one sequence of which has at least 90% and preferably, in order of increasing preference, 95%, 96%, 97%, 98%, 99% and 100% of every continuous stretch of 30 (or fewer, depending on the chosen N-mer size) amino acids present or found in an actual viral isolate, pathogen or cancer sample. In specific embodiments, at least one amino acid sequence is, furthermore, derived from at least three different natural antigen sequences and, in specific embodiments, at least six, and at least ten different natural antigen sequences, in order of increasing preference. In preferred embodiments, the two or more sequences have, in order of increasing preference, less than 70%, 60, and 50% duplicative N-mers or N-mers in common amongst the two or more sequences. In specific embodiments, the resultant consensus sequences are, furthermore, not found in a natural antigen sequence. In specific embodiments the N-mer is a string of amino acids from about 7 to about 30 amino acids. In specific embodiments, the N-mer is selected from the group consisting of: (1) an 8-mer; (2) a 9-mer; (3) a 15-mer; (4) a 16-mer; and (5) a 30-mer.

The present invention also contemplates various compositions comprising at least two consensus antigen sequences. The at least two antigen sequences may, in specific embodiments, be fused. The two or more sequences may further comprise in specific embodiments a sequence between the consensus antigen sequences which comprises a linker or promoter or alternative inclusions

In specific embodiments, the consensus antigen sequence is a viral antigen sequence. The present invention in specific embodiments, provides compositions comprising at least two consensus antigen sequences selected from the group consisting of: gag, nef and pol. In specific embodiments, the compositions comprise amino acid or nucleic acid encoding for existing HIV-1 natural antigen sequences; said antigen sequences, for example, which include without limitation amino acid sequence encoding HIV-1 Gag, Nef and/or Pol, and SEQ ID NO: 46, SEQ ID NO: 80, SEQ ID NO: 100 and/or SEQ ID NO: 112. In specific embodiments, the at least two consensus antigen sequences are (1) HIV-1 gag, nef and pol; (2) HIV-1 gag and nef; (3) HIV-1 nef and pol; and for (4) HIV-1 gag and pol. The present invention also provides in specific embodiments such compositions wherein the at least two consensus antigen sequences are fused, optionally allowing for sequence comprising a linker, promoter or alternative inclusion.

Specific embodiments of the present invention relate to isolated nucleic acid which encodes an HIV antigen(s)/protein(s).

Specific embodiments of the present invention comprise isolated nucleic acid encoding at least one HIV antigen which comprises an amino acid sequence selected from the group consisting of: SEQ ID NO: 1, SEQ ID NO: 2, SEQ ID NO: 3, SEQ ID NO: 4, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 61, SEQ ID NO: 62, SEQ ID NO: 63, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, fusions comprising two or more of the foregoing sequences, and fragments of any of the foregoing sequences; wherein at least 90% (and, in specific embodiments, at least 95%, 96%, 97%, 98%, 99% and 100% in order of increasing preference) of every possible successive N-mer sequence (or sequence of “N” amino acids) of the selected sequence is present in a natural antigen sequence; wherein “N” is any number from about 7 to about 30; and wherein the amino acid sequence selected from the group is not found in a natural antigen sequence. Preferably, and the sequence comprises N-mer sequence from at least three different natural antigen sequences and at least six, and at least ten different natural antigen sequences in preferred embodiments, in order of increasing preference. In specific embodiments, said isolated nucleic acid comprises sequence selected from the group consisting of: SEQ ID NO: 39 (encoding SEQ ID NO; 1); SEQ ID NO: 40 (encoding SEQ ID NO: 2); SEQ ID NO: 41 (encoding SEQ ID NO: 92) and SEQ ID NO: 42 (encoding SEQ ID NO: 93). In specific embodiments, the isolated nucleic acid further comprises nucleic acid encoding HIV-1 Gag, Nef and/or Pol. In specific embodiments, the isolated nucleic acid further comprises nucleic acid encoding SEQ ID NO: 46, SEQ ID NO: 80, SEQ ID NO: 100 or SEQ ID NO: 112.

In specific embodiments, the isolated nucleic acid comprises nucleic acid encoding (a) at least one sequence selected from the group consisting of: SEQ ID NO: 1, SEQ ID NO: 2, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75 and SEQ ID NO: 76; and at least one sequence selected from the group consisting of: SEQ ID NO: 3, SEQ ID NO: 4, SEQ ID NO: 77, SEQ ID NO: 78, SEQ ID NO: 79, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109 and SEQ ID NO: 110. In specific embodiments, the isolated nucleic acid further comprises nucleic acid encoding HIV-1 Gag, Nef and/or Pol. In specific embodiments, the isolated nucleic acid further comprises nucleic acid encoding SEQ ID NO: 46, SEQ ID NO: 80, SEQ ID NO: 100 and/or SEQ ID NO: 112. In specific embodiments, the isolated nucleic acid further comprises SEQ ID NO: 47, SEQ ID NO: 113 and/or SEQ ID NO: 113. In specific embodiments, the isolated nucleic acid comprises two or more sequences from each category. In specific embodiments, the isolated nucleic acid comprises two or more Gag, Nef or Pol consensus antigen sequences. In specific embodiments of the present invention, the two or more sequences may be fused together, optionally comprising a sequence between the consensus antigen sequences which comprises a linker or promoter or alternative inclusions. Specific embodiments of the present invention comprise isolated nucleic acid selected from the group consisting of: SEQ ID NO: 43, SEQ ID NO: 44 and SEQ ID NO: 45.

In specific embodiments, the at least two sequences are selected from (or encode, where applicable) two or more sequences from a set of sequences selected from the group consisting of: (1) SEQ ID NO: 64, SEQ ID NO: 65 and SEQ ID NO: 66; (2) SEQ ID NO: 46, SEQ ID NO: 67 and SEQ ID NO: 68; (3) SEQ ID NO: 69, SEQ ID NO: 70 and SEQ ID NO: 71; (4) SEQ ID NO: 70, SEQ ID NO: 1 and SEQ ID NO: 2; (5) SEQ ID NO: 72, SEQ ID NO: 73 and SEQ ID NO: 74; (6) SEQ ID NO: 70; SEQ ID NO: 75 and SEQ ID NO: 76; (7) SEQ ID NO: 77, SEQ ID NO: 78 and SEQ ID NO: 79; (8) SEQ ID NO: 80, SEQ ID NO: 81 and SEQ ID NO: 82; (9) SEQ ID NO: 83, SEQ ID NO: 84 and SEQ ID NO: 85; (10) SEQ ID NO: 80, SEQ ID NO: 3 and SEQ ID NO: 4; (11) SEQ ID NO: 86, SEQ ID NO: 87 and SEQ ID NO: 88; (12) SEQ ID NO: 80, SEQ ID NO: 89 and SEQ ID NO: 90.

Human Immunodeficiency Virus (“HIV”) is the etiological agent of acquired human immune deficiency syndrome (AIDS) and related disorders. HIV is an RNA virus of the Retroviridae family and exhibits the 5′LTR-gag-pol-env-LTR 3′ organization of all retroviruses. The integrated form of HIV, known as the provirus, is approximately 9.8 Kb in length. Each end of the viral genome contains flanking sequences known as long terminal repeats (LTRs).

Nucleic acid encoding an HIV antigen/protein may be derived from any HIV strain, including but not limited to HIV-1 and HIV-2, strains A, B, C, D, E, F, G, H, I, O, IIIB, LAV, SF2, CM235, and US4; see, e.g., Myers et al., eds. “Human Retroviruses and AIDS: 1995 (Los Alamos National Laboratory, Los Alamos N. Mex. 97545). Another HIV strain suitable for use in the methods disclosed herein is HIV-1 strain CAM-1; Myers et al, eds. “Human Retroviruses and AIDS”: 1995, IIA3-IIA19. This gene closely resembles the consensus amino acid sequence for the clade B (North American/European) sequence. HIV gene sequence(s) may be based on various clades of HIV-1; specific examples of which are Clades A, B, and C. Sequences for genes of many HIV strains are publicly available from GenBank and primary, field isolates of HIV are available from the National Institute of Allergy and Infectious Diseases (NIAID) which has contracted with Quality Biological (Gaithersburg, Md.) to make these strains available. Strains are also available from the World Health Organization (WHO), Geneva Switzerland. Any and all of these genes can form input sequences from which to derive the representative vaccine sequences.

HIV genes are known to encode at least nine proteins which are divided into three classes; the major structural proteins (Gag, Pol, and Env), the regulatory proteins (Tat and Rev); and the accessory proteins (Vpu, Vpr, Vif and Nef). The gag gene encodes a 55-kilodalton (kDa) precursor protein (p55) which is expressed from the unspliced viral mRNA and is proteolytically processed by the HIV protease, a product of the pol gene. The mature p55 protein products are p17 (matrix), p24 (capsid), p9 (nucleocapsid) and p6. The pol gene encodes proteins necessary for virus replication—protease (Pro, P10), reverse transcriptase (RT, P50), integrase (IN, p31) and RNase H(RNase, p15) activities. These viral proteins are expressed as a Gag or Gag-Pol fusion protein which is generated by a ribosomal frame shift. The 55 kDa gag and 160 kDa gagpol precursor proteins are then proteolytically processed by the virally encoded protease into their mature products. The nef gene encodes an early accessory HIV protein (Nef) which has been shown to possess several activities such as down regulating CD4 expression, disturbing T-cell activation and stimulating HIV infectivity. The env gene encodes the viral envelope glycoprotein that is translated as a 160-kilodalton (kDa) precursor (gp160) and then cleaved by a cellular protease to yield the external 120-kDa envelope glycoprotein (gp120) and the transmembrane 41-kDa envelope glycoprotein (gp41). Gp120 and gp41 remain associated and are displayed on the viral particles and the surface of HIV-infected cells. The tat gene encodes a long form and a short form of the Tat protein, a RNA binding protein which is a transcriptional transactivator essential for HIV replication. The rev gene encodes the 13 kDa Rev protein, a RNA binding protein. The Rev protein binds to a region of the viral RNA termed the Rev response element (RRE). The Rev protein promotes transfer of unspliced viral RNA from the nucleus to the cytoplasm. The Rev protein is required for HIV late gene expression and in turn, HIV replication.

Nucleic acid encoding an HIV antigen sequence as well as any consensus antigen sequence described herein may be administered to an individual.

Upon generation of the disclosed antigen consensus sequences, the present invention contemplates, in specific embodiments, the use of codons optimized for expression in mammalian hosts. A “triplet” codon of four possible nucleotide bases can exist in 64 variant forms. That these forms provide the message for only 20 different amino acids (as well as transcription initiation and termination) means that some amino acids can be coded for by more than one codon. Indeed, some amino acids have as many as six “redundant”, alternative codons while some others have a single, required codon. For reasons not completely understood, alternative codons are not at all uniformly present in the endogenous DNA of differing types of cells and there appears to exist variable natural hierarchy or “preference” for certain codons in certain types of cells. As one example, the amino acid leucine is specified by any of six DNA codons, including CTA, CTC, CTG, CTT, TTA, and TTG (which correspond, respectively, to the mRNA codons, CUA, CUC, CUG, CUU, UUA, and UUG). Exhaustive analysis of genome codon frequencies for microorganisms has revealed endogenous DNA of E. coli most commonly contains the CTG leucine-specifying codon, while the DNA of yeasts and slime molds most commonly includes a TTA leucine-specifying codon. In view of this hierarchy, it is generally held that the likelihood of obtaining high levels of expression of a leucine-rich polypeptide by an E. coli host will depend to some extent on the frequency of codon use. For example, a gene rich in TTA codons will in all probability be poorly expressed in E. coli, whereas a CTG rich gene will probably highly express the polypeptide. Similarly, when yeast cells are the projected transformation host cells for expression of a leucine-rich polypeptide, a preferred codon for use in an inserted DNA would be TTA.

The implications of codon preference phenomena on recombinant DNA techniques are manifest, and the phenomenon may serve to explain many prior failures to achieve high expression levels of exogenous genes in successfully transformed host organisms—a less “preferred” codon may be repeatedly present in the inserted gene and the host cell machinery for expression may not operate as efficiently. The phenomenon suggests that synthetic genes which have been designed to include a projected host cell's preferred codons provide a preferred form of foreign genetic material for practice of recombinant DNA techniques; see, e.g., Lathe, 1985, J. Mol. Biol. 183:1-12. For an additional discussion relating to mammalian (human) codon optimization, see WO 97/31115 (PCT/US97/02294). Thus, one aspect of this invention contemplates the delivery and expression of specific HIV genes (including gag, nef and/or pol) which are codon optimized for expression in a human cellular environment.

It is intended that the skilled artisan may use alternative versions of codon optimization or may omit this step when generating antigen and vaccine constructs within the scope of the present invention. Therefore, the present invention also relates to vectors, methods and compositions comprising/utilizing non-codon optimized or partially codon optimized versions of nucleic acid molecules and associated recombinant vector or nucleic acid constructs which encode the antigen consensus sequences. However, codon optimization of these constructs constitutes a preferred embodiment of this invention.

The various codon-optimized forms of nucleic acid encoding the HIV antigen sequences as disclosed herein include codon-optimized HIV gag (including but by no means limited to p55 versions of codon-optimized full length (“FL”) Gag and tPA-Gag fusion proteins), HIV pol, HIV nef, HIV env, HIV tat, HIV rev, and immunologically relevant modifications or derivatives of any of the foregoing. “Immunologically relevant” or “antigenic” as used herein means (1) with regard to an antigen, that the protein is capable, upon administration, of eliciting a measurable immune response within an individual sufficient to retard the propagation and/or spread of the pathogen or cancer and/or to reduce or contain the pathogen or cancer within the individual; or (2) with regards to a nucleotide sequence, that the sequence is capable of encoding for a protein capable of the above.

Specific embodiments contemplated herein encode codon-optimized p55 Gag antigens; codon-optimized Nef antigens; and codon-optimized Pol antigens. Particular sequences may be derived from codon-optimized HIV-1 gag genes as disclosed in PCT

International Application PCT/US00/18332, published Jan. 11, 2001 (WO 01/02607); codon-optimized HIV-1 env genes as disclosed in PCT International Applications PCT/US97/02294 and PCT/US97/10517, published Aug. 28, 1997 (WO 97/31115) and Dec. 24, 1997 (WO 97/48370), respectively; codon-optimized HIV-1 pol genes as disclosed in U.S. application Ser. No. 09/745,221, filed Dec. 21, 2000 and PCT International Application PCT/US00/34724, also filed Dec. 21, 2000; and codon-optimized HIV-1 nef genes as disclosed in U.S. application Ser. No. 09/738,782, filed Dec. 15, 2000 and PCT International Application PCT/US00/34162, also filed Dec. 15, 2000.

The present invention contemplates as well various combinations of antigen sequences derived in accordance with the described methods and antigen sequences not derived by the described methods.

Accordingly, the various codon-optimized sequences referred to herein may be used as the origin sequences (or input sequences) for use in the disclosed methods or as additional sequences to include in the final vaccine or immunogenic constructs. Use in both capacities is disclosed throughout and forms specific embodiments of the present invention. Accordingly, the present invention encompasses specific embodiments which comprise sequences as disclosed herein in combination with available antigen sequences.

A codon-optimized gag gene that can be utilized in the methods and compositions of the present invention is that disclosed in PCT/US00/18332, published Jan. 11, 2001. The sequence is derived from HIV-1 strain CAM-1 and encodes full-length p55 gag. The gag gene of HIV-1 strain CAM-1 was selected as it closely resembles the consensus amino acid sequence for the clade B (North American/European) sequence (Los Alamos HIV database). The sequence was designed to incorporate human preferred (“humanized”) codons in order to maximize in vivo mammalian expression (Lathe, 1985, J. Mol. Biol. 183:1-12).

Codon-optimized pol genes that can be utilized in the methods and compositions of the present invention are disclosed in PCT/US00/34724. Such sequences comprise coding sequences for reverse transcriptase (or RT which consists of a polymerase and RNase H activity) and integrase (IN). Said protein sequences are based on that of Hxb2r, a clonal isolate of IIIB. This sequence has been shown to be closest to the consensus clade B sequence with only 16 nonidentical residues out of 848 (Korber, et al., 1998, Human retroviruses and AIDS, Los Alamos National Laboratory, Los Alamos, N. Mex.).

Particular codon-optimized pol genes that can be utilized in the methods and compositions of the present invention are codon optimized nucleotide sequences which encode wt-pol constructs (herein, “wt-pol” or “wt-pol (codon optimized))” wherein sequences encoding the protease (PR) activity are deleted, leaving codon optimized “wild type” sequences which encode RT (reverse transcriptase and RNase H activity) and IN integrase activity.

Alternative specific embodiments relate to methods and compositions utilizing codon optimized HIV-1 pol wherein, in addition to deletion of the portion of the wild type sequence encoding the protease activity, a combination of active site residue mutations are introduced which are deleterious to HIV-1 pol (RT-RH-IN) activity of the expressed protein. Accordingly, the present invention contemplates in specific embodiments the use of HIV-1 pol wherein the construct is devoid of sequences encoding any PR activity, as well as HIV-1 pol containing a mutation(s) which at least partially, and preferably substantially, abolishes RT, RNase and/or IN activity. One specific type of HIV-1 pol mutant contemplated herein is a mutated nucleic acid molecule comprising at least one nucleotide substitution which results in a point mutation which effectively alters an active site within the RT, RNase and/or IN regions of the expressed protein, resulting in at least substantially decreased enzymatic activity for the RT, RNase H and/or IN functions of HIV-1 Pol. In a specific embodiment of this portion of the invention, a HIV-1 DNA pol construct contains a mutation (or mutations) within the Pol coding region which effectively abolishes RT, RNase H and IN activity. A specific HIV-1 pol-containing construct contains at least one point mutation which alters the active site of the RT, RNase H and IN domains of Pol, such that each activity is at least substantially abolished. Such a HIV-1 Pol mutant will most likely comprise at least one point mutation in or around each catalytic domain responsible for RT, RNase H and IN activity, respectfully. To this end, specific embodiments relate to methods and compositions utilizing HIV-1 pol wherein the encoding nucleic acid comprises nine codon substitution mutations which result in an inactivated Pol protein (IA Pol; as described in PCT/US01/28861, filed Sep. 14, 2001) which has no PR, RT, RNase or IN activity, wherein three such point mutations reside within each of the RT, RNase and IN catalytic domains. Therefore, one exemplification contemplated employs an adenoviral vector construct which comprises, in an appropriate fashion, a nucleic acid molecule which encodes IA-Pol, which contains all nine mutations as shown below in Table 2. An additional amino acid residue for substitution is Asp551, localized within the RNase domain of Pol. Any combination of the mutations disclosed herein may be suitable and therefore may be utilized in the vectors, methods and compositions of the present invention. While addition and deletion mutations are contemplated and within the scope of the invention, the preferred mutation is a point mutation resulting in a substitution of the wild type amino acid with an alternative amino acid residue.

TABLE 2

| | | | enzyme |
|---|---|---|---|
| wt aa | aa residue | mutant aa | function |
| Asp | 112 | Ala | RT |
| Asp | 187 | Ala | RT |
| Asp | 188 | Ala | RT |
| Asp | 445 | Ala | RNase H |
| Glu | 480 | Ala | RNase H |
| Asp | 500 | Ala | RNase H |
| Asp | 626 | Ala | IN |
| Asp | 678 | Ala | IN |
| Glu | 714 | Ala | IN |

It is preferred that point mutations be incorporated into the IApol mutant adenoviral vector constructs so as to lessen the possibility of altering epitopes in and around the active site(s) of HIV-1 Pol. Production of IApol and other gag, nef and/or pol constructs discussed herein is set forth in detail in PCT/US01/28861, filed Sep. 14, 2001.

Particular codon optimized versions of HIV-1 nef and HIV-1 nef modifications of use in specific embodiments of the present invention can be found in U.S. application Ser. No. 09/738,782, filed Dec. 15, 2000 and PCT International Application PCT/US00/34162, also filed Dec. 15, 2000. Particular codon optimized nef and nef modifications relate to nucleic acid encoding HIV-1 Nef from the HIV-1 JRFL isolate wherein the codons are optimized for expression in a mammalian system such as a human. Various DNA molecules which encode this protein can be found in PCT/US01/28861, filed Sep. 14, 2001. One such modified nef optimized coding region codes for modifications at the amino terminal myristylation site (Gly-2 to Ala-2) and substitution of the Leu-174-Leu-175 dileucine motif to Ala-174-Ala-175, forming opt nef (G2A, LLAA). Yet another modified nef optimized coding region has modifications at the amino terminal myristylation site (Gly-2 to Ala-2), forming opt nef (G2A). Antigen sequences with these changes are found in specific embodiments comprising: SEQ ID NOs: 92-93 and 97-110. Specific embodiments of fusion proteins comprising these sequences comprise: SEQ ID NOs: 94-96.

HIV-1 Nef is a 216 amino acid cytosolic protein which associates with the inner surface of the host cell plasma membrane through myristylation of Gly-2 (Franchini et al., 1986, Virology 155: 593-599). While not all possible Nef functions have been elucidated, it has become clear that correct trafficking of Nef to the inner plasma membrane promotes viral replication by altering the host intracellular environment to facilitate the early phase of the HIV-1 life cycle and by increasing the infectivity of progeny viral particles. In one aspect of the invention, the methods, vectors and compositions of the present invention have therein codon-optimized nef sequence that is modified to contain a nucleotide sequence which encodes a heterologous leader peptide such that the amino terminal region of the expressed protein will contain the leader peptide.

The diversity of function that typifies eukaryotic cells depends upon the structural differentiation of their membrane boundaries. To generate and maintain these structures, proteins must be transported from their site of synthesis in the endoplasmic reticulum to predetermined destinations throughout the cell. This requires that the trafficking proteins display sorting signals that are recognized by the molecular machinery responsible for route selection located at the access points to the main trafficking pathways. Sorting decisions for most proteins need to be made only once as they traverse their biosynthetic pathways since their final destination, the cellular location at which they perform their function, becomes their permanent residence. Maintenance of intracellular integrity depends in part on the selective sorting and accurate transport of proteins to their correct destinations. Defined sequence motifs exist in proteins which can act as ‘address labels’. A number of sorting signals have been found associated with the cytoplasmic domains of membrane proteins. An effective induction of CTL responses often requires sustained, high level endogenous expression of an antigen. As membrane-association via myristylation is an essential requirement for most of Nef's function, mutants lacking myristylation, by glycine-to-alanine change, change of the dileucine motif and/or by substitution with a leader sequence, will be functionally defective, and therefore will have improved safety profile compared to wild-type Nef for use as an HIV-1 vaccine component.

Accordingly, specific embodiments of the present invention contemplate vaccine constructs comprising a eukaryotic trafficking signal peptide or a leader peptide such as that found in highly expressed mammalian proteins such as immunoglobulin leader peptides. It is well within the realm of one skilled in the art to test any functional leader peptide for efficacy and employ same in the vectors, compositions and methods of the present invention. Known recombinant DNA methodology may be used to incorporate desired sequences into the various constructs.

Nucleic acid as referred to herein may be DNA and/or RNA, and may be double or single stranded. The nucleic acid may be in the form of an expression cassette. In this respect, specific embodiments of the present invention relate to a gene expression cassette comprising (a) nucleic acid as described herein encoding a protein or antigen of interest; (b) a heterologous promoter operatively linked to the nucleic acid encoding the protein/antigen; and (c) a transcription termination signal.

In specific embodiments, the heterologous promoter is recognized by a eukaryotic RNA polymerase. One example of a promoter suitable for use in the present invention is the immediate early human cytomegalovirus promoter (Chapman et al., 1991 Nucl. Acids Res. 19:3979-3986). Further examples of promoters that can be used in the present invention are the immunoglobulin promoter, the EF1 alpha promoter, the murine CMV promoter, the Rous Sarcoma Virus promoter, the SV40 early/late promoters and the beta actin promoter, albeit those of skill in the art can appreciate that any promoter capable of effecting expression of the heterologous nucleic acid in the intended host can be used in accordance with the methods of the present invention. The promoter may comprise a regulatable sequence such as the Tet operator sequence. Sequences such as these that offer the potential for regulation of transcription and expression are useful in circumstances where repression/modulation of gene transcription is sought. The gene expression cassette may comprise a transcription termination sequence; specific embodiments of which are the bovine growth hormone termination/polyadenylation signal (bGHpA) or the short synthetic polyA signal (SPA) of 49 nucleotides in length defined as follows: AATAAAAGATCTTTATTTTCATTAGATCTGTGTGTTGGTTTTTTGTGTG (SEQ ID NO: 114). A leader or signal peptide may also be incorporated into the transgene. In specific embodiments, the leader is derived from the tissue-specific plasminogen activator protein, tPA.

Another aspect of the present invention relates to the various vectors and compositions comprising the disclosed vaccine antigen sequences.

Vectors of use in the methods and compositions of the present invention may comprise one or more sequences as described herein. The administration of at least one (preferably, at least two) vector(s) comprising two or more antigen sequences, their derivatives, or modifications are anticipated. Two or more antigen sequences may be expressed on at least one of the recombinant vector constructs and/or two or more antigen sequences may be expressed across two or more constructs. One of skill in the art can readily appreciate that the present invention, therefore, encompasses those situations where, while only one antigen may be in common amongst at least two vectors, the vectors may have additional antigen sequences that (1) differ, (2) are the same, (3) while not in common with that vector, are in common with another vector utilized in the disclosed methods or compositions, or (4) are derived from the same common antigen. Therefore, the present invention offers the possibility of using the methods and compositions of the present invention to effectuate a multi-valent antigen administration, specific examples, but not limitations of which, include the administration of adenoviral vectors comprising nucleic acid sequence encoding (1) Gag and Nef polypeptides, (2) Gag and Pol polypeptides, (3) Pol and Nef polypeptides, and (4) Gag, Pol and Nef polypeptides.

Multiple genes/encoding nucleic acid may be ligated into a plasmid or shuttle plasmid for generation of the ultimate construct. This is of interest with, for example, adenoviral vectors where multiple genes/encoding nucleic acid may be ligated into a shuttle plasmid for generation of a pre-adenoviral plasmid comprising multiple open reading frames.

Open reading frames for the multiple genes/encoding nucleic acid may be operatively linked to distinct promoters and transcription termination sequences. In other embodiments, the open reading frames may be operatively linked to a single promoter, with the open reading frames operatively linked by an internal ribosome entry sequence (IRES; as disclosed in WO 95/24485), or suitable alternative allowing for transcription of the multiple open reading frames to run off of a single promoter. In certain embodiments, the open reading frames may be fused together by stepwise PCR or suitable alternative methodology for fusing together two open reading frames. Various combined modality administration regimens suitable for use in the present invention are disclosed in PCT/US01/28861, published Mar. 21, 2002.

Selection of the administration vehicle or vector, be it viral, nucleic acid (e.g., as a plasmid), protein or other, is not deemed critical to the successful practice hereof. Any vehicle capable of delivering the antigen(s) (or effectuating expression of the antigen(s)) to sufficient levels such that a cellular and/or humoral-mediated response is elicited is sufficient and forms an important embodiment of the present invention.

Suitable viral vehicles include but are not limited to the various serotypes of adenovirus, including but not limited to adenovirus serotypes 5, 6, 24, 26, 34, 35 and various modification and derivatives thereof. Additional viral vehicles suitable for administration of the disclosed vaccine antigen sequences include adeno-associated virus (“AAV”; see, e.g., Samulski et al., 1987 J. Virol. 61:3096-3101; Samulski et al., 1989 J. Virol. 63:3822-3828); retrovirus (see, e.g., Miller, 1990 Human Gene Ther. 1:5-14; Ausubel et al., Current Protocols in Molecular Biology); pox virus (including but not limited to replication-impaired NYVAC, ALVAC, TROVAC and MVA vectors, see, e.g., Panicali & Paoletti, 1982 Proc. Natl. Acad. Sci. USA 79:4927-31; Nakano et al. 1982 Proc. Natl. Acad. Sci. USA 79: 1593-1596; Piccini et al., In Methods in Enzymology 153:545-63 (Wu & Grossman, eds., Academic Press, San Diego); Sutter et al., 1994 Vaccine 12:1032-40; Wyatt et al., 1996 Vaccine 15:1451-8; and U.S. Pat. Nos. 4,603,112; 4,769,330; 4,722,848; 4,603,112; 5,110,587; 5,174,993; and 5,185,146); and alpha virus (see, e.g., WO 92/10578; WO 94/21792; WO 95/07994; and U.S. Pat. Nos. 5,091,309 and 5,217,879).

Various polynucleotide administrations are contemplated herein, including but not limited to “naked DNA” or facilitated polynucleotide delivery); see, e.g., Wolff et al., 1990 Science 247:1465, and the following patent publications: U.S. Pat. Nos. 5,580,859; 5,589,466; 5,739,118; 5,736,524; 5,679,647; WO 90/11092 and WO 98/04720.

A specific embodiment of the present invention relates to the use of adenoviruses as the delivery vehicle. Adenoviruses are nonenveloped, icosahedral viruses that have been identified in several avian and mammalian hosts; Horne et al., 1959 J. Mol. Biol. 1:84-86; Horwitz, 1990 In Virology, eds. B. N. Fields and D. M. Knipe, pp. 1679-1721. The first human adenoviruses (Ads) were isolated over four decades ago. Since then, over 100 distinct adenoviral serotypes have been isolated which infect various mammalian species, 51 of which are of human origin; Straus, 1984, In The Adenoviruses, ed. H. Ginsberg, pps. 451-498, New York: Plenus Press; Hierholzer et al., 1988 J. Infect. Dis. 158:804-813; Schnurr and Dondero, 1993, Intervirology; 36:79-83; De Jong et al., 1999 J Clin Microbiol., 37:3940-5. The human serotypes have been categorized into six subgenera (A-F) based on a number of biological, chemical, immunological and structural criteria which include hemagglutination properties of rat and rhesus monkey erythrocytes, DNA homology, restriction enzyme cleavage patterns, percentage G+C content and oncogenicity; Straus, supra; Horwitz, supra. These various adenoviral serotypes may be utilized in the methods/compositions of the present invention. One of skill in the art can readily identify and develop adenoviruses of alternative and distinct serotype (including, but not limited to, the foregoing) for purposes consistent with the methods and compositions of the present invention. Those of skill in the art are, furthermore, readily familiar with the various adenoviral serotypes including, but not limited to, (1) the numerous serotypes of subgenera A-F discussed above, (2) unclassified adenovirus serotypes, (3) non-human serotypes (including but not limited to primate adenoviruses (see, e.g., Fitzgerald et al., 2003 J. Immunol. 170 (3) 1416-1422; Xiang et al., 2002 J. Virol. 76(6):2667-2675)), and equivalents, modifications, or derivatives of the foregoing. Adenoviruses can readily be obtained from the American Type Culture Collection (“ATCC”) or other publicly available/private source; and adenoviral sequences can be discerned from both the published literature and widely accessible public databases, where not obtained elsewhere.

The present invention also relates in specific embodiments to compositions comprising at least two adenoviral serotypes; said at least two adenoviral serotypes comprising heterologous nucleic acid encoding at least one common polypeptide; as described in International Publication No. WO 06/020480, published Feb. 23, 2006. Accordingly, the present invention contemplates in specific embodiments the contemporaneous administration of adenovirus serotypes 5 and 6, both encoding at least one common polypeptide of interest. Adenovirus serotypes 5 and 6 are well known in the art (American Type Culture Collection (“ATCC”) Deposit Nos. VR-5 and VR-6, respectively, and sequences therefore have been published; see Chroboczek et al., 1992 J. Virol. 186:280, and PCT/US02/32512, published Apr. 17, 2003, respectively).

In preferred embodiments, adenoviruses are rendered replication-defective through deletion or modification of the essential early-region 1 (“E1”) of the viral genomes. This results in viruses that are devoid (or essentially devoid) of E1 activity and, thus, incapable of replication in the intended host/vaccinee; see, e.g., Brody et al, 1994 Ann N Y Acad. Sci., 716:90-101. Preferably, the E1 region is completely deleted or inactivated. Deletion of adenoviral genes other than E1 (e.g., in E2, E3 and/or E4), furthermore, creates adenoviral vectors with greater capacity for heterologous gene inclusion. Specific embodiments of the present invention employ adenoviral vectors as described in PCT/US01/28861, published Mar. 21, 2002. Said vectors are at least partially deleted in E1 and comprise several adenoviral packaging repeats (i.e., the E1 deletion does not start until approximately base pairs 450-458, with base pair numbers assigned corresponding to a wildtype Ad5 sequence). The adenoviruses may contain additional deletions in E3, and other early regions, albeit in certain situations where E2 and/or E4 is deleted, E2 and/or E4 complementing cell lines may be required to generate recombinant, replication-defective adenoviral vectors. Vectors devoid of adenoviral protein-coding regions (“gutted vectors”) are also feasible for use herein. Such vectors typically require the presence of helper virus for the propagation and development thereof.

Construction of adenoviral vectors may be accomplished using techniques well understood and appreciated in the art, such as those reviewed in Graham & Prevec, 1991 In Methods in Molecular Biology: Gene Transfer and Expression Protocols, (Ed. Murray, E. J.), p. 109; and Hitt et al., 1997 “Human Adenovirus Vectors for Gene Transfer into Mammalian Cells” Advances in Pharmacology 40:137-206.

E1-complementing cell lines used for the propagation and rescue of recombinant adenovirus should provide elements essential for the viruses to replicate, whether the elements are encoded in the cell's genetic material or provided in trans. It is, furthermore, preferable that the E1-complementing cell line and the vector not contain overlapping elements which could enable homologous recombination between the nucleic acid of the vector and the nucleic acid of the cell line potentially leading to replication competent virus (or replication competent adenovirus “RCA”). Often, propagation cells are human cells derived from the retina or kidney, although any cell line capable of expressing the appropriate E1 and any other critical deleted region(s) can be utilized to generate adenovirus suitable for use in the methods of the present invention. Embryonal cells such as amniocytes have been shown to be particularly suited for the generation of E1 complementing cell lines. Several cell lines are available and include but are not limited to the known cell lines PER.C6® (Crucell, Leiden, The Netherlands, ECACC deposit number 96022940), 911, 293, and E1 A549. PER.C6® cell lines are described in WO 97/00326 (published Jan. 3, 1997) and issued U.S. Pat. No. 6,033,908. PER.C6® is a primary human retinoblast cell line transduced with an E1 gene segment that complements the production of replication deficient (FG) adenovirus, but is designed to prevent generation of replication competent adenovirus by homologous recombination. 293 cells are described in Graham et al., 1977 J. Gen. Virol. 36:59-72. For the propagation and rescue of non-group C adenoviral vectors, a cell line expressing an E1 region which is complementary to the E1 region deleted in the virus being propagated can be utilized. Alternatively, a cell line expressing regions of E1 and E4 derived from the same serotype can be employed; see, e.g., U.S. Pat. No. 6,270,996. Another alternative would be to propagate non-group C adenovirus in available E1-expressing cell lines (e.g., PER.C6®, A549 or 293). This latter method involves the incorporation of a critical E4 region into the adenovirus to be propagated. The critical E4 region is native to a virus of the same or highly similar serotype as that of the E1 gene product(s) (particularly the E1B 55K region) of the complementing cell line, and comprises typically, at a minimum, E4 open reading frame 6 (“ORF6”)); see, PCT/US2003/026145, published Mar. 4, 2004. One of skill in the art can readily appreciate and carry out numerous other methods suitable for the production of recombinant, replication-defective adenoviruses suitable for use in the methods of the present invention. Following viral production in whatever means employed, viruses may be purified, formulated and stored prior to host administration.

In addition to the delivery of nucleic acid in the various means described, the present invention contemplates as well, in specific embodiments, the administration of purified or recombinant protein. In this respect, recombinant (i.e., derived by man) polypeptides comprising the disclosed amino acid sequences and encoded by disclosed nucleotide sequences form specific embodiments of the present invention. In specific embodiments the recombinant polypeptides comprise at least one sequence selected from the group consisting of: SEQ ID NO: 1, SEQ ID NO: 2, SEQ ID NO: 3, SEQ ID NO: 4, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 61, SEQ ID NO: 62, SEQ ID NO: 63, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, fusions comprising two or more of the foregoing sequences, and fragments of any of the foregoing sequences; wherein at least 90% (and, in specific embodiments, at least 95%, 96%, 97%, 98%, 99% and 100% in order of increasing preference) of every possible successive sequence of “N” amino acids (“N-mer” sequence) is present in a natural antigen sequence; wherein “N” is any number from about 7 to about 30; and wherein the amino acid sequence selected from the group is not found in a natural antigen sequence. In specific embodiments, the recombinant polypeptide further comprises an amino acid sequence encoding a natural antigen sequence for Gag, Nef and/or Pol. In specific embodiments, the recombinant polypeptide further comprises SEQ ID NO: 46, SEQ ID NO: 80, SEQ ID NO: 100 and/or SEQ ID NO: 112. In specific embodiments, the at least one sequence comprises N-mer sequence from at least three different natural antigen sequences and, in additional specific embodiments, from at least six, and from at least ten different natural antigen sequences, in order of increasing preference. As the skilled artisan will no doubt appreciate, a greater number of sequences factored in or included in the dataset enhances the effectiveness of the consensus sequences for eliciting a broadly reactive immune response. This is because the expressed proteins, through the presentation of epitopes representative of various different natural strains or sequences, are capable of eliciting a more broadly cross-reactive immune response.

In specific embodiments, the recombinant polypeptide comprises (a) at least one sequence selected from the group consisting of: SEQ ID NO: 1, SEQ ID NO: 2, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75 and SEQ ID NO: 76; and at least one sequence selected from the group consisting of: SEQ ID NO: 3, SEQ ID NO: 4, SEQ ID NO: 77, SEQ ID NO: 78, SEQ ID NO: 79, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109 and SEQ ID NO: 110. In specific embodiments, the recombinant polypeptide further comprises an amino acid sequence for Gag, Nef and/or Pol. In specific embodiments, the recombinant polypeptide further comprises SEQ ID NO: 46, SEQ ID NO: 80, SEQ ID NO: 100 and/or SEQ ID NO: 112. In specific embodiments, the recombinant polypeptide comprises two or more amino acid sequences from each category. In specific embodiments, the recombinant polypeptide comprises two or more Gag, Nef or Pol consensus antigen sequences. In specific embodiments of the present invention, the two or more sequences may be fused together, optionally comprising a sequence between the consensus antigen sequences which comprises a linker or promoter or alternative inclusions.

Recombinant protein may be produced by any method available to the skilled artisan including, but not limited to, through direct synthesis or via various recombinant expression techniques available (for instance, in yeast, E. coli, or any other suitable expression system). In specific embodiments, the polypeptide of the invention may be prepared by culturing transformed host cells under culture conditions suitable to express the recombinant polypeptide. The resulting expressed polypeptide may then be purified from such culture (i.e., from culture medium or cell extracts) using known purification processes including, but not limited to, gel filtration and ion exchange chromatography. Purified, recombinant polypeptides form specific embodiments of the present invention. The polypeptide thus purified is substantially free of other mammalian polypeptides other than those polypeptides affirmatively adjoined or added after or during purification and is defined in accordance with the present invention as an “isolated polypeptide” or “recombinant polypeptide”; such isolated or recombinant polypeptides of the invention include polypeptides of the invention, fragments, and variants.

One specific embodiment of the present invention contemplates an immunization regime that employs simultaneous delivery of isolated nucleic acid and recombinant protein. In alternative embodiments, the nucleic acid delivery and protein administration form part of a prime-boost administration; where the nucleic acid delivery either precedes or follows recombinant protein delivery. Recombinant protein could be produced by any method available to the skilled artisan including, but not limited to, through direct synthesis or via various recombinant expression techniques available (for instance, in yeast, E. coli, or any other suitable expression system).

The present invention further encompasses cells, populations of cells, and non-human transgenic animals comprising the nucleic acid, vectors and/or antigens described herein.

Additional embodiments of the present invention are compositions comprising nucleic acid, viral or other vehicles comprising said nucleic acid, or recombinant polypeptides encoded by said nucleic acid. In particular embodiments, the compositions comprise purified replication-defective adenovirus particles comprising nucleic acid encoding an antigen sequence wherein every successive N-mer sequence is present in a natural antigen sequence. Particular embodiments are compositions comprising purified replication-defective adenovirus particles comprising nucleic acid encoding a viral antigen sequence wherein every possible 16-mer extract of the sequence can be traced to an actual natural antigen sequence. Additional embodiments of the present invention relate to compositions comprising recombinant or purified polypeptide expressed by nucleic acid as disclosed herein.

Compositions comprising the recombinant antigen vehicles or vectors may contain physiologically acceptable components, such as buffer, normal saline or phosphate buffered saline, sucrose, other salts and polysorbate. The pharmaceutically acceptable carrier may also be selected from any excipient, diluent, stabilizer, buffer, or alternative designed to facilitate administration of the antagonist in the desired amount to the treated individual. The pharmaceutical carrier, further, may be a sterile liquid, such as water and oil. Some examples of suitable pharmaceutical carriers are described in “Remington's Pharmaceutical Sciences” by E. W. Martin.

In specific embodiments the viral particles are formulated in A195 formulation buffer. See U.S. Patent Application Publication No. 2005/0186225 A1. In certain embodiments, the formulation has: 2.5-10 mM TRIS buffer, preferably about 5 mM TRIS buffer; 25-100 mM NaCl, preferably about 75 mM NaCl; 2.5-10% sucrose, preferably about 5% sucrose; 0.01-2 mM MgCl₂; and 0.001%-0.01% polysorbate 80 (plant derived). The pH should range from about 7.0-9.0, preferably about 8.0. One skilled in the art will appreciate that other conventional vaccine excipients may also be used in the formulation. In specific embodiments, the formulation contains 5 mM TRIS, 75 mM NaCl, 5% sucrose, 1 mM MgCl₂, 0.005% polysorbate 80 at pH 8.0. This has a pH and divalent cation composition which is near the optimum for virus stability and minimizes the potential for adsorption of virus to glass surface. It does not cause tissue irritation upon intramuscular injection. It is preferably frozen until use.

The amount of delivery vehicle to be used in the vaccine composition(s) ultimately introduced into a vaccine recipient will depend on the strength of the transcriptional and translational promoters used and on the immunogenicity of the expressed gene product(s). For purposes of illustration, an immunologically or prophylactically effective dose of 1×10⁷ to 1×10¹² adenoviral particles and preferably about 1×10¹⁰ to 1×10¹¹ adenoviral particles is administered directly into muscle tissue.

Administration of additional agents able to potentiate or broaden the immune response (e.g., the various cytokines, interleukins), concurrently with or subsequent to parenteral introduction of the viral vectors of this invention is appreciated herein as well and can be advantageous.

All methods and compositions described herein are well suited to effectuate an immune response that will recognize the particular virus, bacteria, cancer antigen or alternative antigen of interest, because any particular epitope expressed upon introduction of the vaccine constructs into an individual will be derivable from a natural antigen sequence. Accordingly, specific embodiments of the present invention comprise the delivery and expression of heterologous nucleic acid encoding a polypeptide(s) of interest, particularly heterologous nucleic acid encoding an antigen sequence wherein every successive N-mer sequence is present in a natural antigen sequence. Particular embodiments relate to the delivery and expression of heterologous nucleic acid encoding a polypeptide(s) of interest, particularly heterologous nucleic acid encoding a viral antigen sequence wherein every possible 16-mer extract of the sequence can be traced to an actual natural antigen sequence. Additional embodiments of the present invention relate to the administration of recombinant or purified polypeptides expressed by nucleic acid as disclosed herein.

The disclosed antigen sequences, corresponding antigens, constructs, compositions and methods as described herein should, thus, more broadly and effectively impact the transmission rate to or occurrence rate in previously uninfected or unimpacted individuals (i.e., prophylactic applications) and/or the levels of virus/bacteria/foreign agent/cancer within an infected or impacted individual (i.e., therapeutic applications).

Accordingly, methods of using the various nucleic acid and polypeptide compositions for eliciting cellular-mediated immune or immunological responses specific for the antigens form additional, important embodiments of the present invention.

Regardless of the antigen/method chosen, contemporaneous administration of delivery vehicles is contemplated for specific embodiments of the present invention. Prime-boost regimens can employ different viruses (including but not limited to different viral serotypes and viruses of different origin), viral vector/protein combinations, and combinations of viral and polynucleotide administrations. In one type of scenario, for instance, an individual may first be administered a priming dose of a protein/antigen/derivative/modification utilizing a certain vehicle (be that a viral vehicle, purified and/or recombinant protein, or encoding nucleic acid). Multiple primings, typically 1-4, are usually employed, although more may be used. The priming dose(s) effectively primes the immune response so that, upon subsequent identification of the protein/antigen(s) in the circulating immune system, the immune response is capable of immediately recognizing and responding to the protein/antigen(s) within the host. Following some period of time, the individual is administered a boosting dose of at least one of the previously delivered protein(s)/antigen(s), derivatives or modifications thereof (administered by viral vehicle/protein/nucleic acid). The length of time between priming and boost may typically vary from about four months to a year, albeit other time frames may be used as one of ordinary skill in the art will appreciate. The follow-up or boosting administration may also be repeated at selected time intervals. In certain embodiments, contemporaneous administration in accordance herewith can be employed for both the prime and boost administrations. A mixed modality prime and boost inoculation scheme should result in an enhanced immune response, specifically where there is pre-existing anti-vector immunity.

Various administration regimes are contemplated. Subcutaneous injection, intradermal introduction, impression through the skin, and other modes of administration such as intraperitoneal, intravenous, or inhalation delivery are also contemplated. One of ordinary skill in the art can also appreciate that the different modes of administration can be tailored to the particular delivery vehicle employed. Additionally, one of ordinary skill in the art will appreciate that combinations of vehicles may use distinct administration modes and specifics.

Potential hosts/vaccinees/individuals that can benefit from the described administrations include but are not limited to primates and especially humans and non-human primates, and include any non-human mammal of commercial or domestic veterinary importance.

Compositions as described herein may also be administered as part of a broader treatment regimen. The present invention, thus, encompasses those situations where the disclosed antigen constructs are administered in conjunction with other therapies; including but not limited to other antimicrobial (e.g., antiviral, antibacterial) agent treatment therapies or anti-cancer therapies. The particular antimicrobial agent(s) or anti-cancer therapy selected is not critical to the successful practice of the methods disclosed herein. The antimicrobial agent or anti-cancer therapy can, for example, be based on/derived from an antibody, a polynucleotide, a polypeptide, a peptide, or a small molecule. Any antimicrobial agent or anti-cancer therapy that effectively reduces microbial replication/spread/load or controls the spread or impacts the integrity of a cancer within an individual is sufficient for the uses described herein.

Antiviral agents antagonize the functioning/life cycle of a virus, and target a protein/function essential to the proper life cycle of the virus; an effect that can be readily determined by an in vivo or in vitro assay. Some representative antiviral agents which target specific viral proteins are protease inhibitors, reverse transcriptase inhibitors (including nucleoside analogs; non-nucleoside reverse transcriptase inhibitors; and nucleotide analogs), and integrase inhibitors. Protease inhibitors include, for example, indinavir/CRIXIVAN® (Merck & Co., Inc, Whitehouse, N.J.); ritonavir/NORVIR® (Abbott Laboratories, Abbott Park, Ill.); saquinavir/FORTOVASE® (Hoffmann-LaRoche Inc., Nutley, N.J.); nelfinavir/VIRACEPT® (Agouron Pharmaceuticals, LaJolla, Calif.); amprenavir/AGENERASE® (Glaxo Group Ltd. Corp., Middlesex, U.K.); lopinavir and ritonavir/KALETRA® (Abbott). Reverse transcriptase inhibitors include, for example, (1) nucleoside analogs, e.g., zidovudine/RETROVIR® (GSK) (AZT); didanosine/VIDEX® (Bristol-Myers Squibb, Princeton, N.J.) (ddI); stavudine/ZERIT® (BMS) (d4T); lamivudine/EPIVIR® (GSK) (3TC); abacavir/ZIAGEN® (GSK) (ABC); (2) non-nucleoside reverse transcriptase inhibitors, e.g., nevirapine/VIRAMUNE® (Boehringer Ingelheim Corp., Ridgefield, Conn.) (NVP); delavirdine/RESCRIPTOR® (Pfizer, New York, N.Y.) (DLV); efavirenz/SUSTIVA® (BMS) (EFV); and (3) nucleotide analogs, e.g., tenofovir DF/VIREAD® (Gilead Sciences, Foster City, Calif.) (TDF). Integrase inhibitors include, for example, the molecules disclosed in U.S. Application Publication No. US2003/0055071, published Mar. 20, 2003; and International Application WO 03/035077. The antiviral agents, as indicated, can target as well a function of the virus/viral proteins, such as, for instance the interaction of regulatory proteins tat or rev with the trans-activation response region (“TAR”) or the rev-responsive element (“RRE”), respectively. An antiviral agent is, preferably, selected from the class of compounds consisting of: a protease inhibitor, an inhibitor of reverse transcriptase, and an integrase inhibitor. Preferably, the antiviral agent administered to an individual is some combination of effective antiviral therapeutics such as that present in highly active anti-retroviral therapy (“HAART”), a term generally used in the art to refer to a cocktail of inhibitors of viral protease and reverse transcriptase.

One of skill in the art can, furthermore, appreciate that the present invention can be employed in conjunction with any pharmaceutical composition useful for the treatment of microbial infections or cancer. Antimicrobial agents and cancer therapies are typically administered in their conventional dosage ranges and regimens as reported in the art, including the dosages described in the Physicians' Desk Reference, 54^{th} edition, Medical Economics Company, 2000.

The following non-limiting examples are presented to better illustrate the workings of the invention.

### Example 1

### Input Data

Sequences were downloaded from the Los Alamos National Laboratory (LANL) HIV Sequence Database, a curated set of sequences that are also available in GenBank. Amino acid translations in all three reading frames were imported into a FileMaker (FileMaker, Inc., Santa Clara, Calif.) database. Sequences that failed to span at least 90% of the defined length of the HXB2 standard sequence were eliminated. Each remaining amino acid sequence was aligned and manually validated by inspection and the sequence derived from the correct reading frame was identified by comparison with the sequence of HXB2. Sequences with internal frameshifts were identified by multiple alignment and omitted from the working data set. Sequences with many ambiguous bases or those tagged as problematic by the LANL HIV database were eliminated. Only sequences having patient identification codes were retained. Sequences determined in-house from HIV-1-infected patient samples were added to those obtained from the LANL HIV database. For these, at least five independent clones were sequenced from each patient sample. For each individual, their sequences were assigned to a single HIV clade according to similarity of those sequences to HIV clade-specific archetype sequences, using the genotyping tool available from the National Center for Biotechnology Information (NCBI; Bethesda, Md.) and accessible on their website.

The final sequences were analyzed according to the algorithm as disclosed herein.

The following vaccine sequences were, thus, designed to maximize the number of potential epitopes in HIV infections.

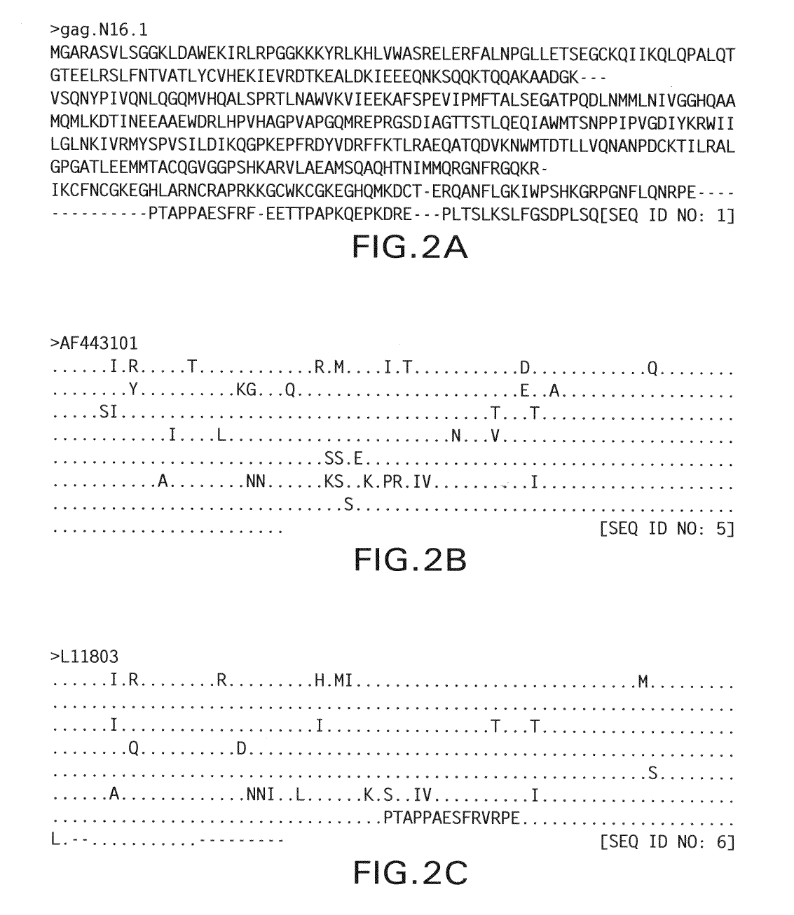

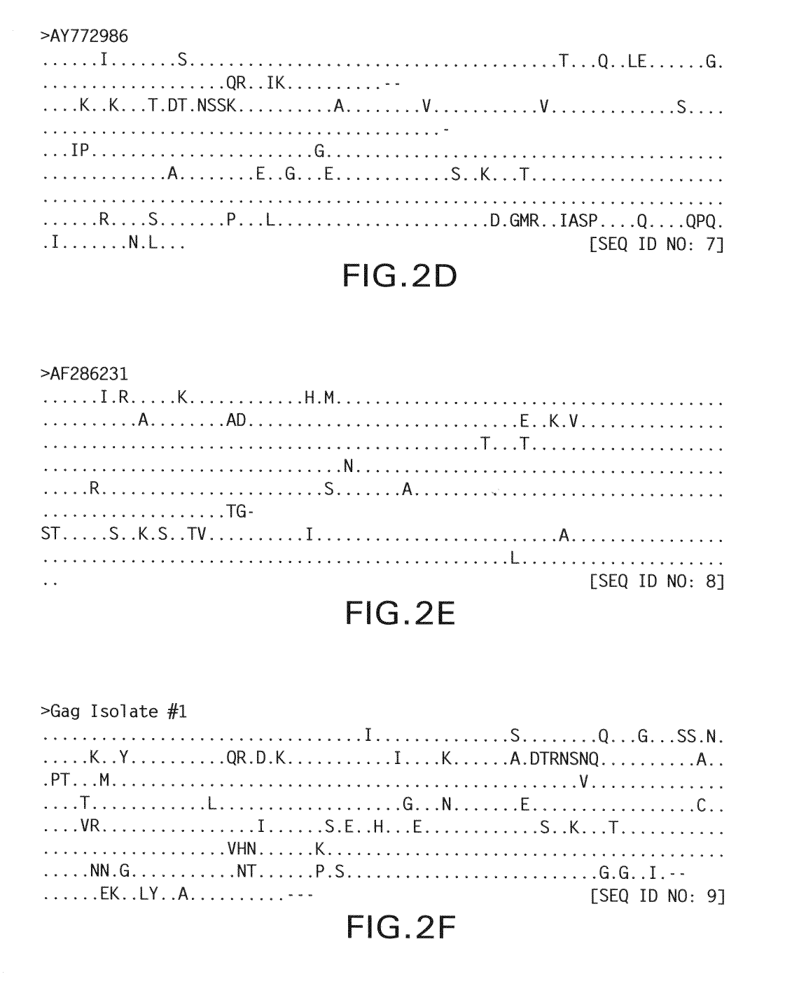

Sequence gag.N16.1 (SEQ ID NO: 1, FIG. 2A; an encoding nucleic acid provided as SEQ ID NO: 39) is designed to optimize 16mer coverage. Said sequence can be used in conjunction with clade B gag (CAM1) described in PCT International Application No. PCT/US01/28861, filed Sep. 14, 2001 and, in specific embodiments, be included in a single vaccine.

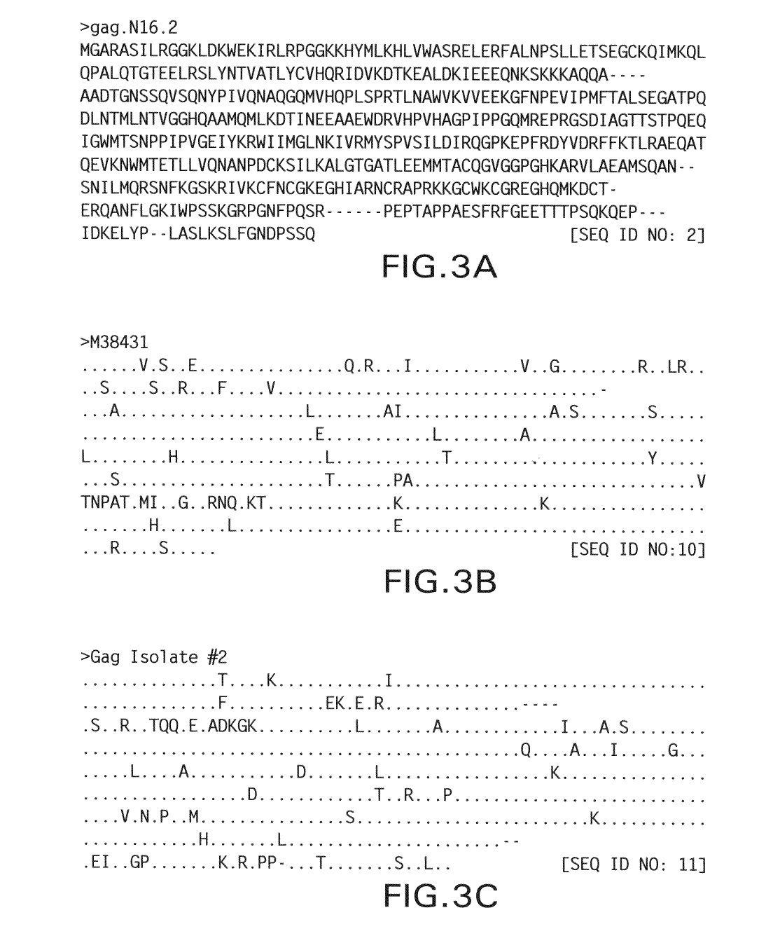

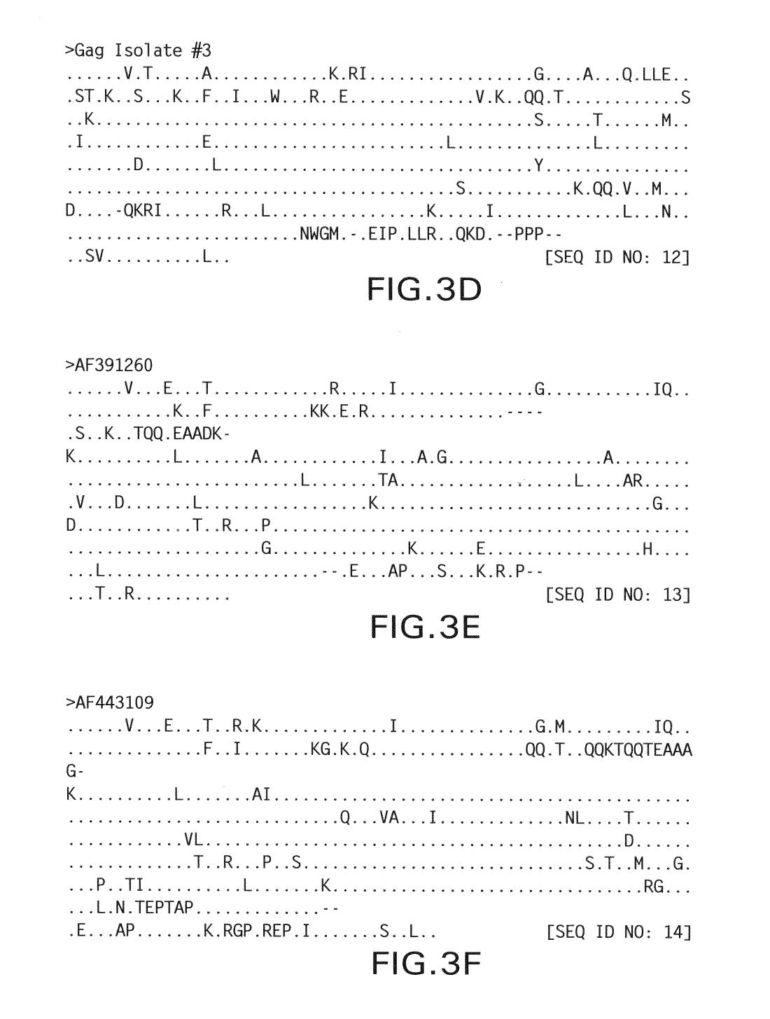

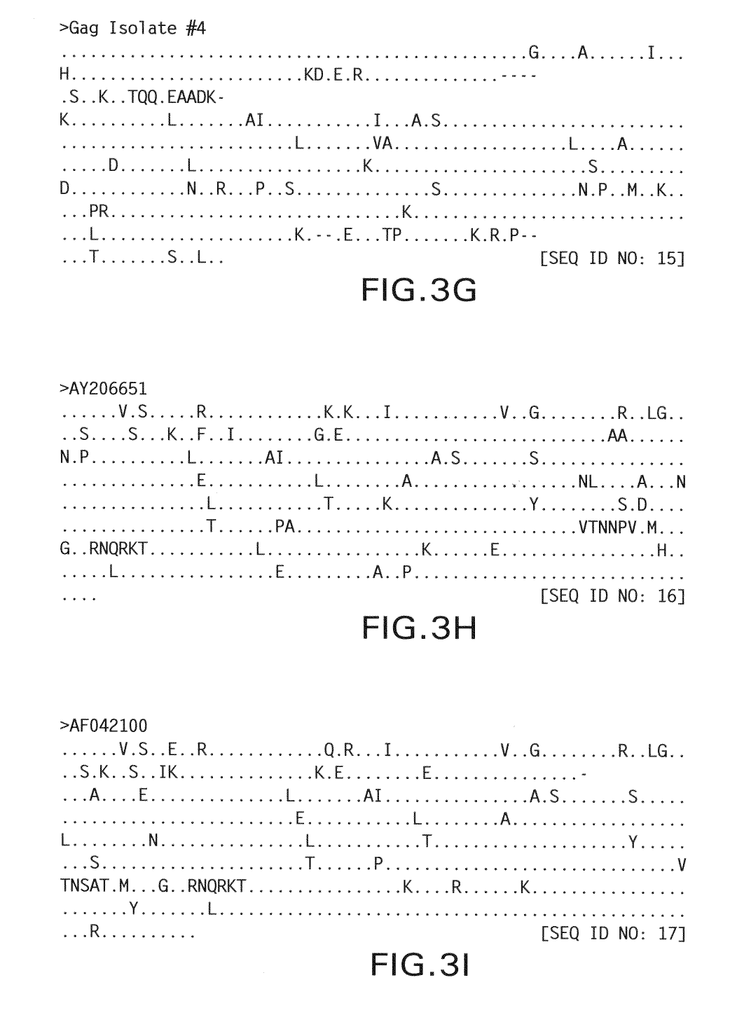

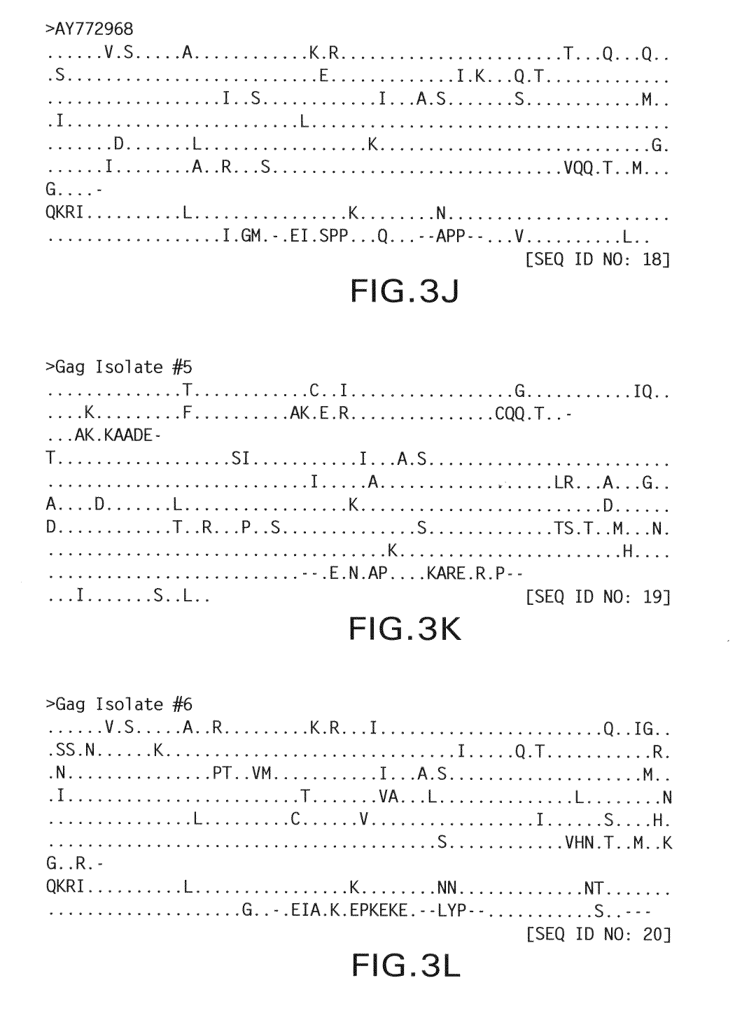

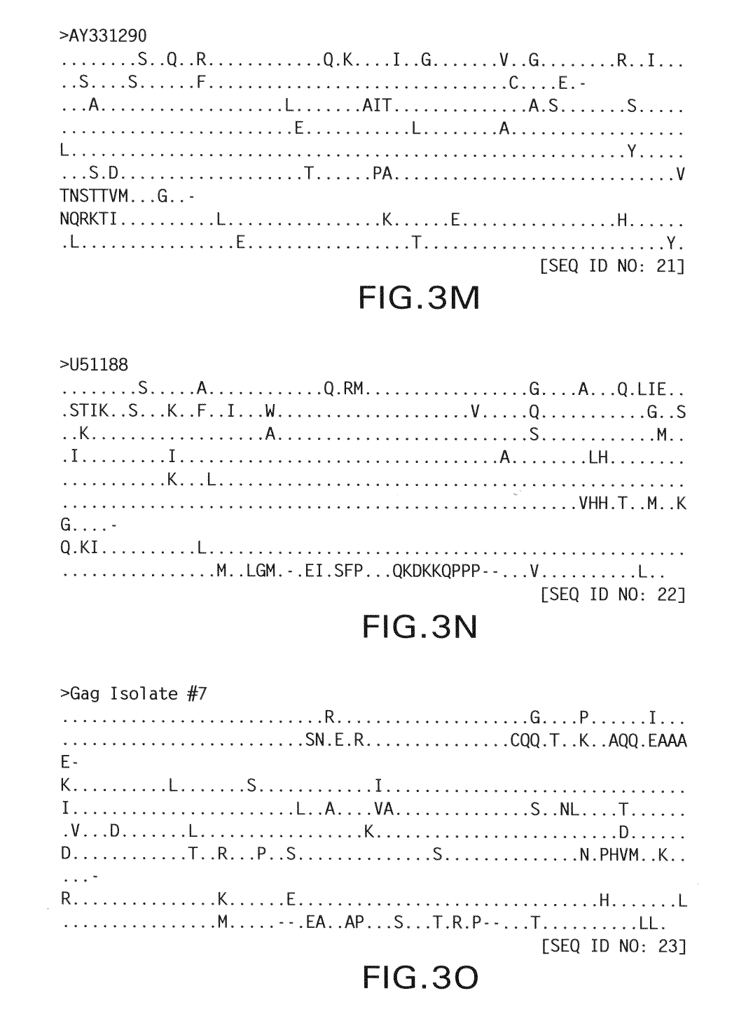

Sequence gag.N16.2 (SEQ ID NO: 2, FIG. 3A; an encoding nucleic acid provided as SEQ ID NO: 40) is designed to optimize 16mer coverage. Said sequence can be used in conjunction with clade B gag (CAM1) described in PCT International Application No. PCT/US01/28861, filed Sep. 14, 2001; and Sequence gag.N16.1 and, in specific embodiments, be included in a single vaccine with either one or both.

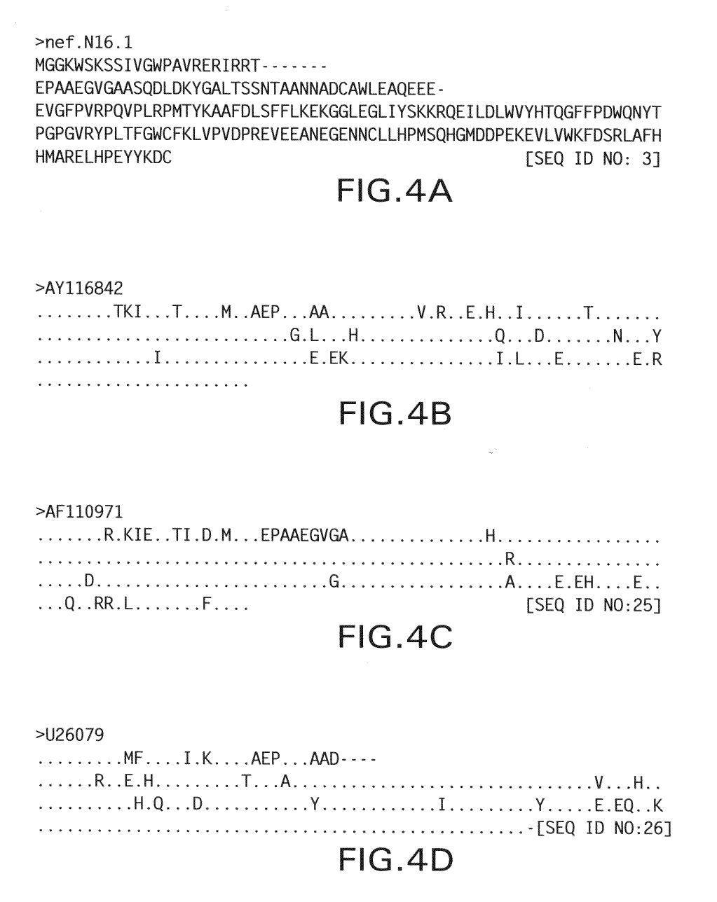

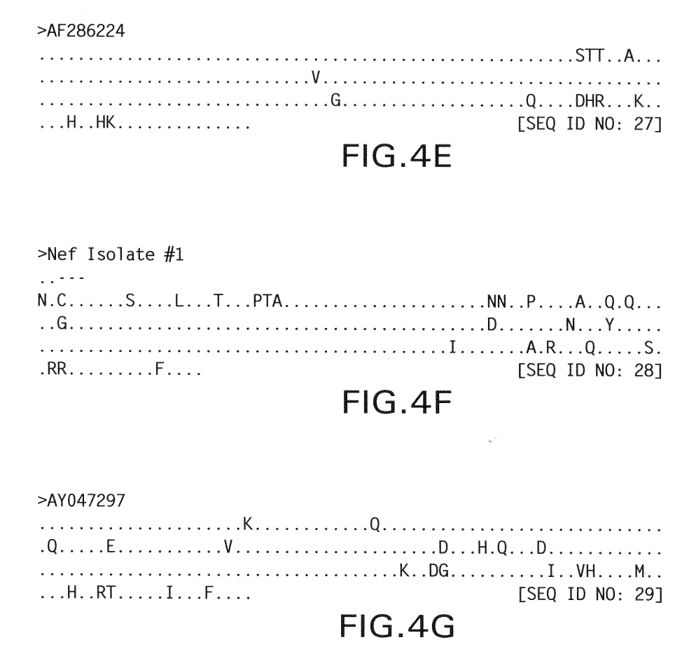

Sequence nef.N16.1 (SEQ ID NO: 92; FIG. 4A) is designed to optimize 16mer coverage. Said sequence can be used in conjunction with clade B nef (JRFL) described in PCT International Application No. PCT/US01/28861, filed Sep. 14, 2001 and, in specific embodiments, be included in a single vaccine.

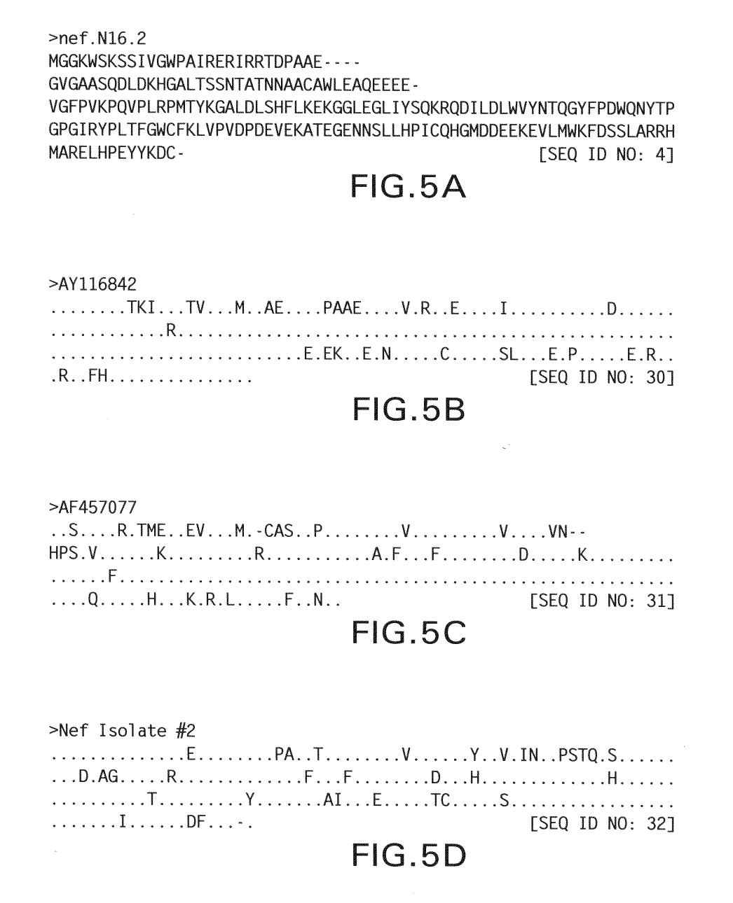

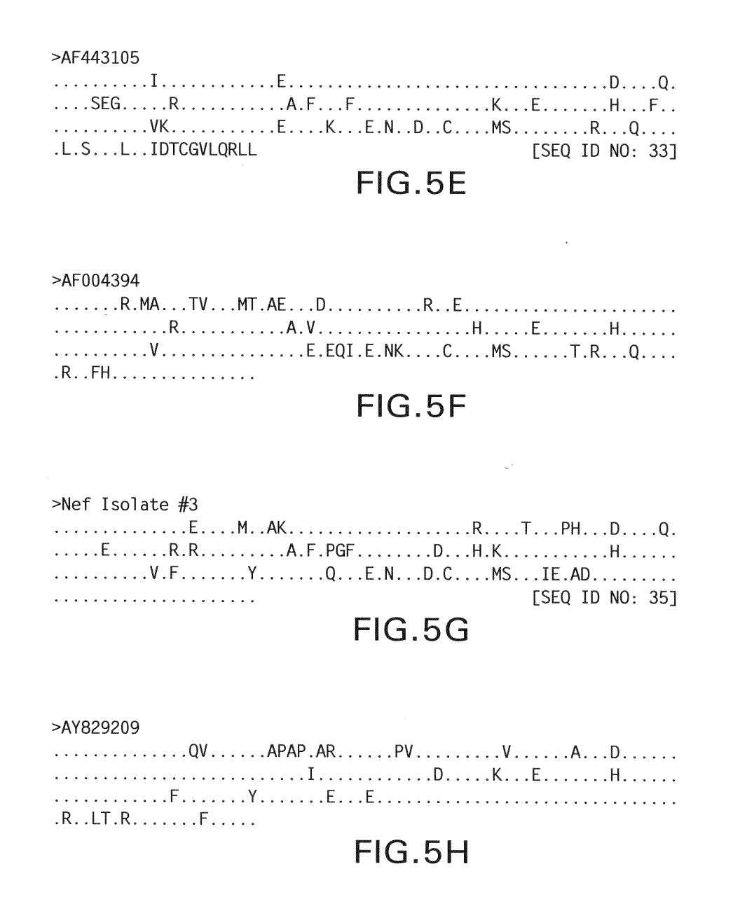

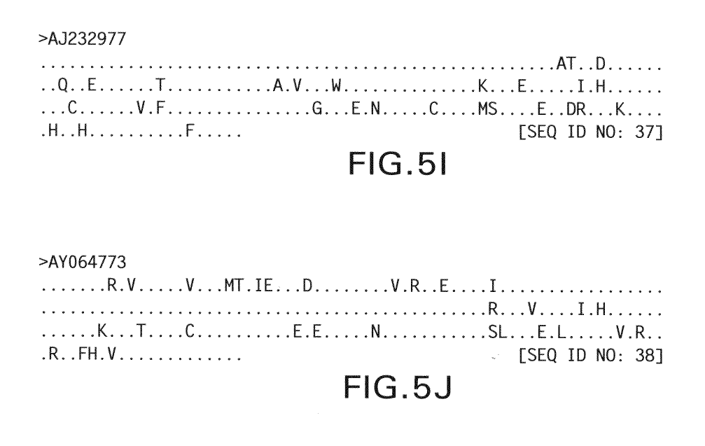

Sequence nef.N16.2 (SEQ ID NO: 93; FIG. 5A) is designed to optimize 16mer coverage. Said sequence can be used in conjunction with clade B nef (JRFL) described in PCT International Application No. PCT/US01/28861, filed Sep. 14, 2001; and Sequence nef.N16.1 and, in specific embodiments, be included in a single vaccine with either one or both.

Human CD8 epitopes may range from 7 to 14 amino acids, with typical ranges being from 9 to 10 amino acids. The number of amino acids for CD4+ (helper) epitopes has been reported to range from 9 amino acids in length to as long as 20 amino acids in length, with typical ranges from 15-16 amino acids. The above sequences are composed of 16-mer amino acid fragments from present-day HIV-1 viral isolates found in infected humans. The fragments were combined into a single continuous sequence such that any 16-mer extract of the sequences can be traced to at least one actual viral isolate (and, in practice, many isolates). In the process, no artificial epitopes are created nor are real epitopes abrogated by these sequences. In particular, the 16-mers that comprise the sequence are chosen to maximize the total overlap with the global set of HIV-1 viral sequences. These sequences are, additionally, weighted such that all patients contribute equally, and clades are represented according to their estimated global prevalence, irrespective of their arbitrary frequency in the database itself.

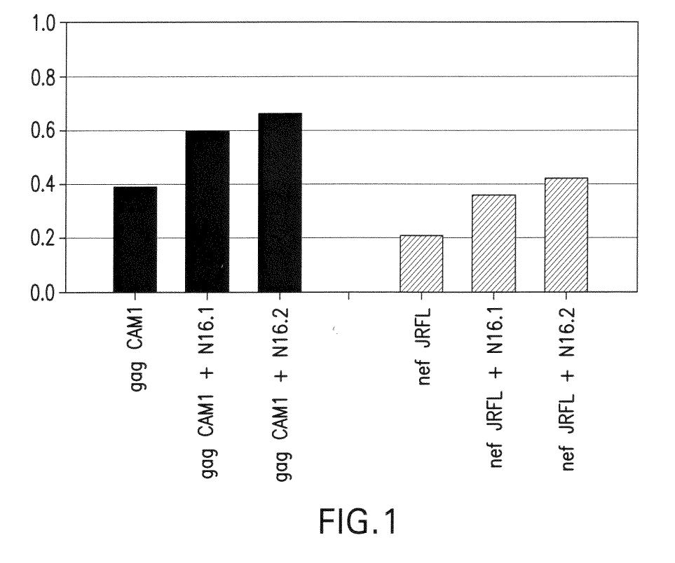

As illustrated in FIG. 1, the overall number of breadth of global coverage increases significantly over the unsupplemented Gag CAM1 or Nef JRFL alone.

### Example 2

### Construction of an Ad5 Vector Containing an HIV-1 Gag-Gag-Nef-Nef Fusion Transgene

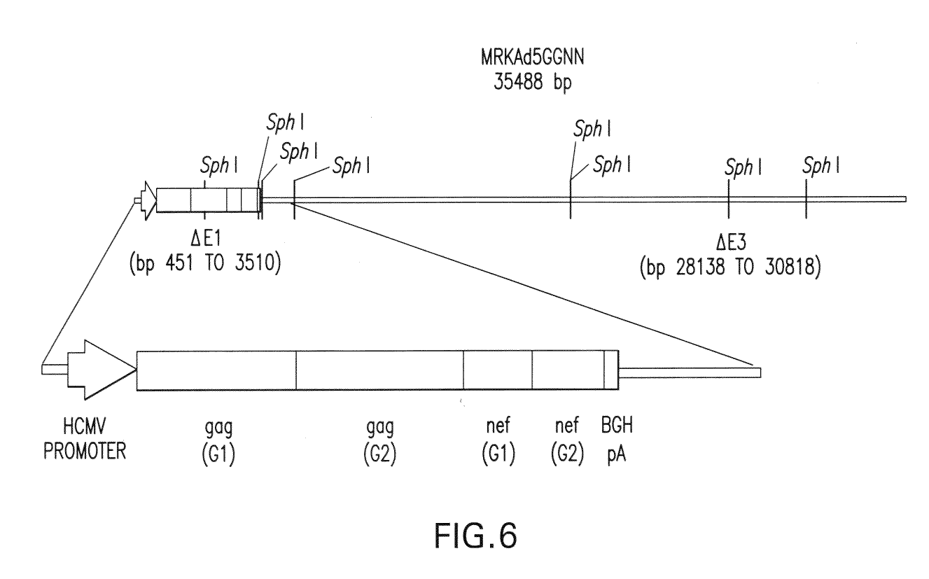

MRKAd5GGNN is depicted in FIG. 6. The vector is a modification of a prototype Group C Ad5 whose genetic sequence has been reported previously; Chroboczek et al., 1992 J. Virol. 186:280-285. The E1 region of the wild-type Ad5 (nt 451-3510) is deleted and replaced with the transgene. The transgene contains the gag-gag-nef-nef expression cassette consisting of 1) the immediate early gene promoter from the human cytomegalovirus; Chapman et al., 1991 Nucl. Acids Res. 19:3979-3986, 2) the coding sequence of the human immunodeficiency virus type 1 (HIV-1) gag global 1 gene fused to gag global 2, fused to nef global 1, fused to nef global 2 (amino acid sequence provided as SEQ ID NO: 94; an encoding nucleic acid sequence provided as SEQ ID NO: 43), and 3) the bovine growth hormone polyadenylation signal sequence; Goodwin & Rottman, 1992 J. Biol. Chem. 267:16330-16334. The amino acid sequence of the gaggagnefnef protein was generated from Example 1. Codons were selected to optimize expression in human cells (R. Lathe, 1985 J. Mol. Biol. 183:1-12) and to reduce regions of homology within the coding sequences. No more than 12 consecutive base pairs (bp) are homologous between the two gag or two nef coding sequences. The gag open reading frames encode the matrix, capsid, and nucleocapsid proteins. The nef open reading frames were altered by mutating the myristoylation site located at Gly-2 to an alanine. This mutation prevents attachment of nef to the cytoplasmic membrane and retrotrafficking into endosomes, thereby functionally inactivating nef; W. Pandori et al., 1996 J. Virol. 70:4283-4290. In addition to the deletion of the E1 region, the vector has an E3 deletion (nt 28138 to 30818) in order to accommodate the transgene.

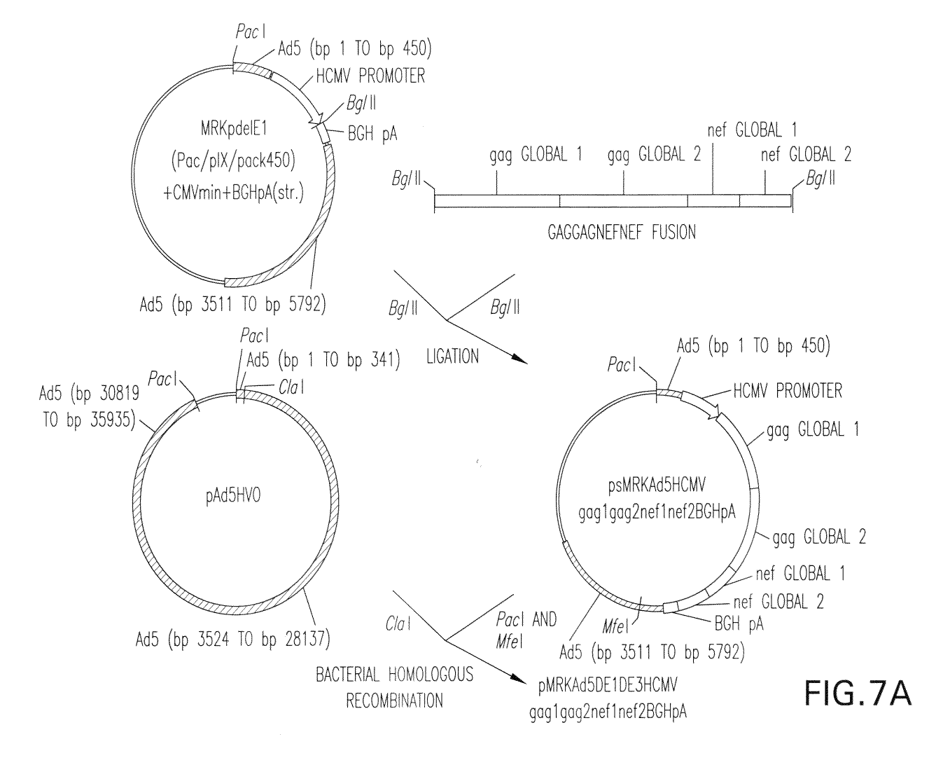

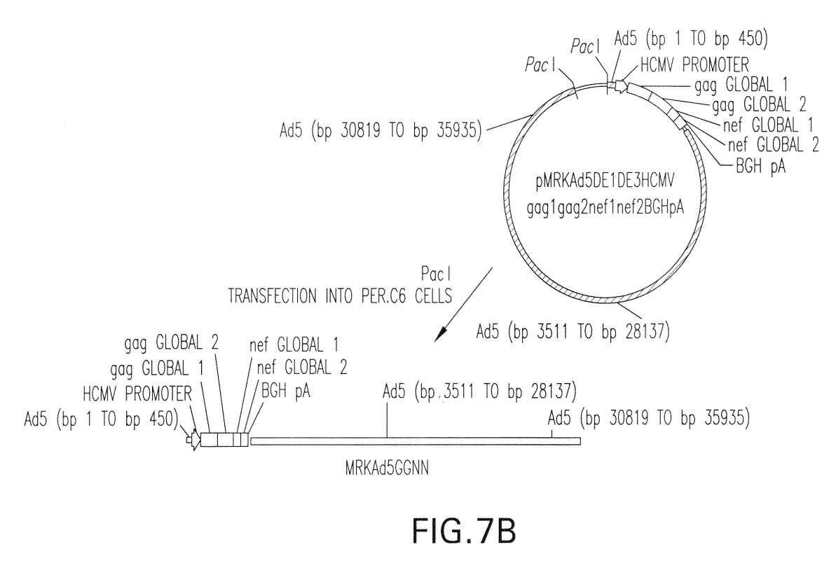

Key steps involved in the construction of MRKAd5GGNN are depicted in FIGS. 7A-B and described in the text that follows.

### (1) Construction of Adenoviral Shuttle Vector:

The shuttle plasmid psMRKAd5HCMVgag1gag2nef1nef2BGHpA was constructed by inserting a synthetic full-length codon-optimized HIV-1 gaggagnefnef fusion gene into pMRKdelE1 (Pac/pIX/pack450)+CMVmin+BGHpA (str.). The synthetic full-length codon-optimized HIV-1 gaggagnefnef gene was synthesized at DNA2.0, Inc. (Menlo Park, Calif.). The synthesized gene was ligated into the BglII restriction endonuclease site in MRKpdelE1 (Pac/pIX/pack450)+CMVmin+BGHpA (str.), generating plasmid psMRKAd5HCMVgag1gag2nef1nef2BGHpA. The genetic structure of psMRKAd5HCMVgag1gag2nef1nef2BGHpA was verified by restriction enzyme and DNA sequence analyses.

### (2) Construction of Pre-Adenovirus Plasmid:

To construct pre-adenovirus pMRKAd5DE1DE3HCMVgag1gag2nef1nef2BGHpA, the transgene containing fragment was liberated from shuttle plasmid psMRKAd5HCMVgag1gag2nef1nef2BGHpA by digestion with restriction enzymes PacI and MfeI and gel purified. The purified transgene fragment was then co-transformed into E. coli strain BJ5183 with linearized (ClaI-digested) adenoviral backbone plasmid, pAd5HVO (also referred to as pAd5E1-E3-). Plasmid DNA isolated from BJ5183 transformants was then transformed into competent E. coli XL-1 Blue for screening by restriction analysis. The desired plasmid pMRKAd5DE1DE3HCMVgag1gag2nef1nef2BGHpA was verified by restriction enzyme digestion and DNA sequence analysis.

### (3) Generation of Recombinant MRKAd5GGNN:

To prepare virus the pre-adenovirus plasmid pMRKAd5DE1DE3HCMVgag1gag2nef1nef2BGHpA was rescued as infectious virions in PER.C6® adherent monolayer cell culture. To rescue infectious virus, 10 μg of pMRKAd5DE1DE3HCMVgag1gag2nef1nef2BGHpA was digested with restriction enzyme PacI (New England Biolabs) and then transfected into one T25 flask of PER.C6® cells using the calcium phosphate co-precipitation technique. PacI digestion releases the viral genome from plasmid sequences, allowing viral replication to occur after entry into PER.C6® cells. Infected cells and media were harvested 10 days post-transfection, after complete viral cytopathic effect (CPE) was observed. The virus stock was amplified by 2 passages in PER.C6® cells. At passage 2, virus was purified on CsCl density gradients. To verify that the rescued virus had the correct genetic structure, viral DNA was isolated and analyzed by restriction enzyme (SphI and BglII) analysis. The rescued virus was referred to as MRKAd5GGNN (also called MRKAd5DE1DE3HCMVgag1gag2nef1nef2BGHpA).

### Example 3

### Construction of an Ad5 Vector Containing an HIV-1 Gag-Nef-Gag-Nef Fusion Transgene

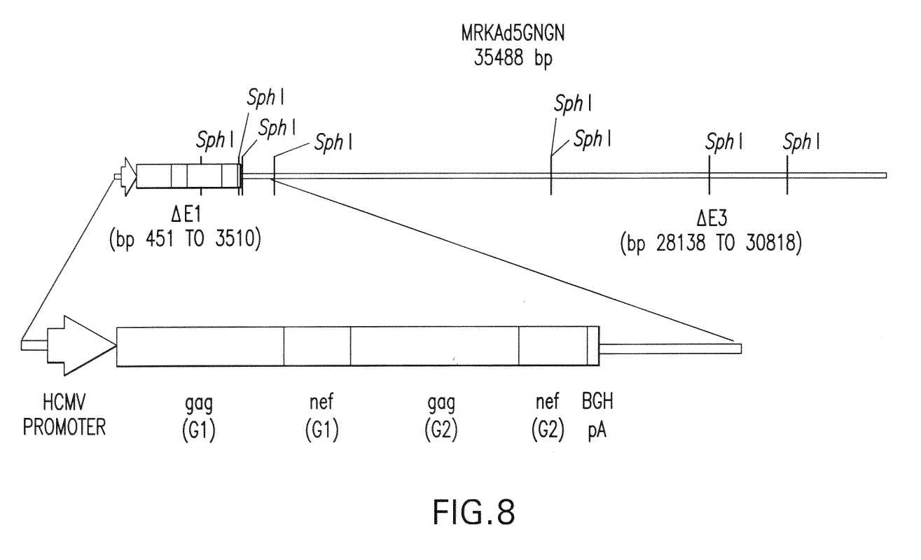

MRKAd5GNGN is depicted in FIG. 8. The vector is a modification of a prototype Group C Ad5 whose genetic sequence has been reported previously; Chroboczek et al., 19921 Virol. 186:280-285. The E1 region of the wild-type Ad5 (nt 451-3510) is deleted and replaced with the transgene. The transgene contains the gag-nef-gag-nef expression cassette consisting of: 1) the immediate early gene promoter from the human cytomegalovirus; Chapman et al., 1991 Nucl. Acids Res. 19:3979-3986, 2) the coding sequence of the human immunodeficiency virus type 1 (HIV-1) gag global 1 gene fused to nef global 1, fused to gag global 2, fused to nef global 2 (amino acid sequence provided as SEQ ID NO: 96; an encoding nucleic acid sequence provided as SEQ ID NO: 44), and 3) the bovine growth hormone polyadenylation signal sequence; Goodwin & Rottman, 1992 J. Biol. Chem. 267:16330-16334. The amino acid sequence of the gagnefgagnef protein was generated from Example 1. Codons were selected to optimize expression in human cells (R. Lathe, 1985 J. Mol. Biol. 183:1-12) and to reduce regions of homology within the coding sequences. No more than 12 consecutive bp's are homologous between the two gag or two nef coding sequences. The gag open reading frames encode the matrix, capsid, and nucleocapsid proteins. The nef open reading frames were altered by mutating the myristoylation site located at Gly-2 to an alanine. This mutation prevents attachment of nef to the cytoplasmic membrane and retrotrafficking into endosomes, thereby functionally inactivating nef; W. Pandori et al., 1996 J. Virol. 70:4283-4290. In addition to the deletion of the E1 region, the vector has an E3 deletion (nt 28138 to 30818) in order to accommodate the transgene.

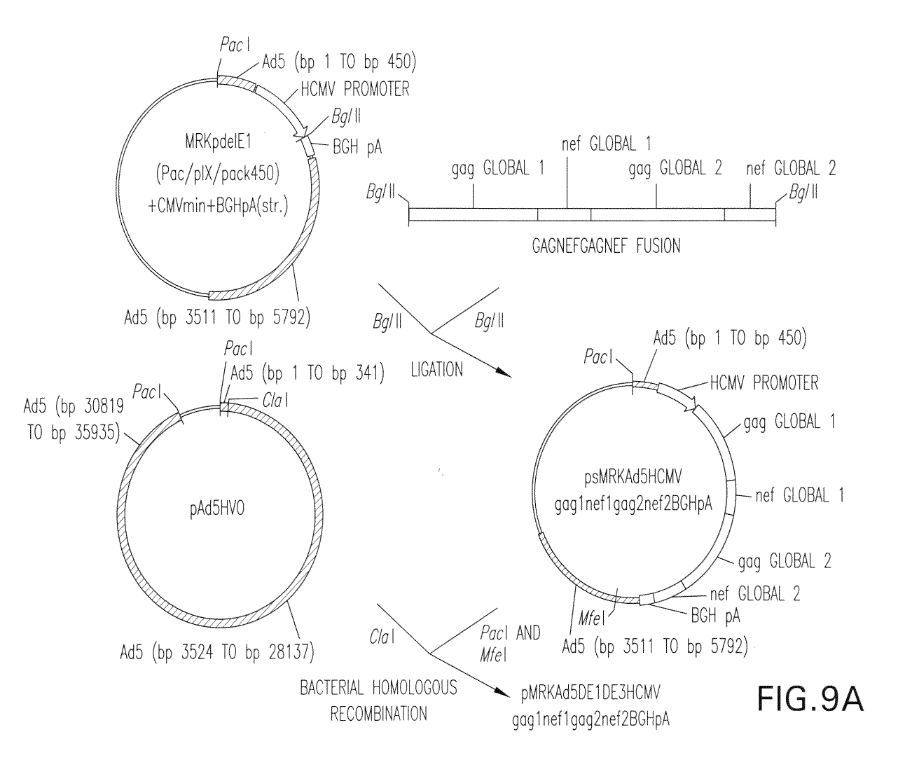

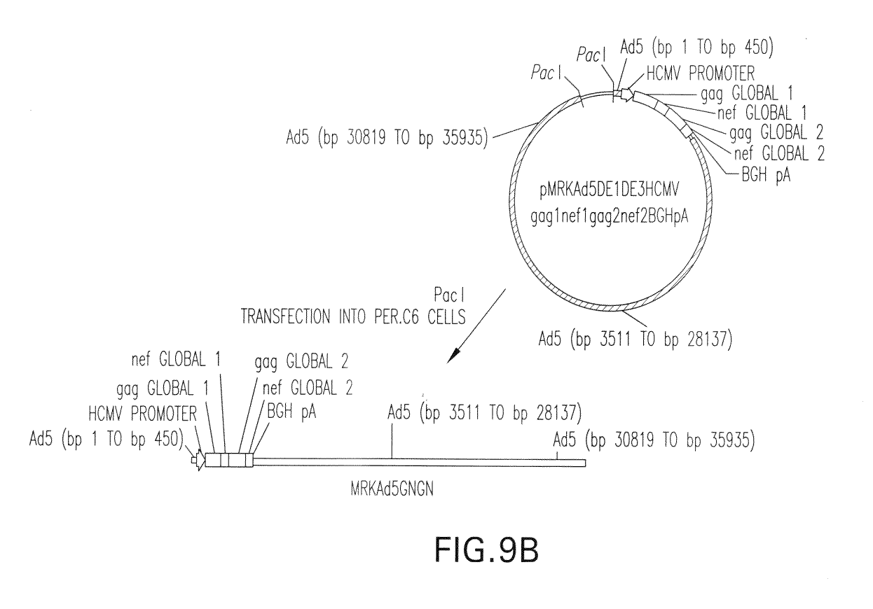

Key steps involved in the construction of MRKAd5GNGN are depicted in FIGS. 9A-B and described in the text that follows.

### (1) Construction of Adenoviral Shuttle Vector:

The shuttle plasmid psMRKAd5HCMVgag1nef1gag2nef2BGHpA was constructed by inserting a synthetic full-length codon-optimized HIV-1 gagnefgagnef fusion gene into MRKpdelE1 (Pac/pIX/pack450)+CMVmin+BGHpA (str.). The synthetic full-length codon-optimized HIV-1 gagnefgagnef gene was synthesized at DNA2.0. The synthesized gene was ligated into the BglII restriction endonuclease site in MRKpdelE1 (Pac/pIX/pack450)+CMVmin+BGHpA (str.), generating plasmid psMRKAd5HCMVgag1nef1gag2nef2BGHpA. The genetic structure of psMRKAd5HCMVgag1nef1gag2nef2BGHpA was verified by restriction enzyme and DNA sequence analyses.

### (2) Construction of Pre-Adenovirus Plasmid:

To construct pre-adenovirus pMRKAd5DE1DE3HCMVgag1nef1gag2nef2BGHpA, the transgene containing fragment was liberated from shuttle plasmid psMRKAd5HCMVgag1nef1gag2nef2BGHpA by digestion with restriction enzymes PacI and MfeI and gel purified. The purified transgene fragment was then co-transformed into E. coli strain BJ5183 with linearized (ClaI-digested) adenoviral backbone plasmid, pAd5HVO (also referred to as pAd5E1-E3-). Plasmid DNA isolated from BJ5183 transformants was then transformed into competent E. coli XL-1 Blue for screening by restriction analysis. The desired plasmid pMRKAd5DE1DE3HCMVgag1nef1gag2nef2BGHpA was verified by restriction enzyme digestion and DNA sequence analysis.

### (3) Generation of Recombinant MRKAd5GNGN:

To prepare virus the pre-adenovirus plasmid pMRKAd5DE1DE3HCMVgag1nef1gag2nef2BGHpA was rescued as infectious virions in PER.C6® adherent monolayer cell culture. To rescue infectious virus, 10 μg of pMRKAd5DE1DE3HCMVgag1nef1gag2nef2BGHpA was digested with restriction enzyme PacI (New England Biolabs) and then transfected into one T25 flask of PER.C6® cells using the calcium phosphate co-precipitation technique. PacI digestion releases the viral genome from plasmid sequences, allowing viral replication to occur after entry into PER.C6® cells. Infected cells and media were harvested 10 days post-transfection, after complete viral cytopathic effect (CPE) was observed. The virus stock was amplified by 2 passages in PER.C6® cells. At passage 2, virus was purified on CsCl density gradients. To verify that the rescued virus had the correct genetic structure, viral DNA was isolated and analyzed by restriction enzyme (SphI and BglII) analysis. The rescued virus was referred to as MRKAd5GNGN (also called MRKAd5DE1DE3HCMVgag1nef1gag2nef2BGHpA).

### Example 4

### Construction of an Ad6 Vector Containing an HIV-1 Gag-Gag-Nef-Nef Fusion Transgene

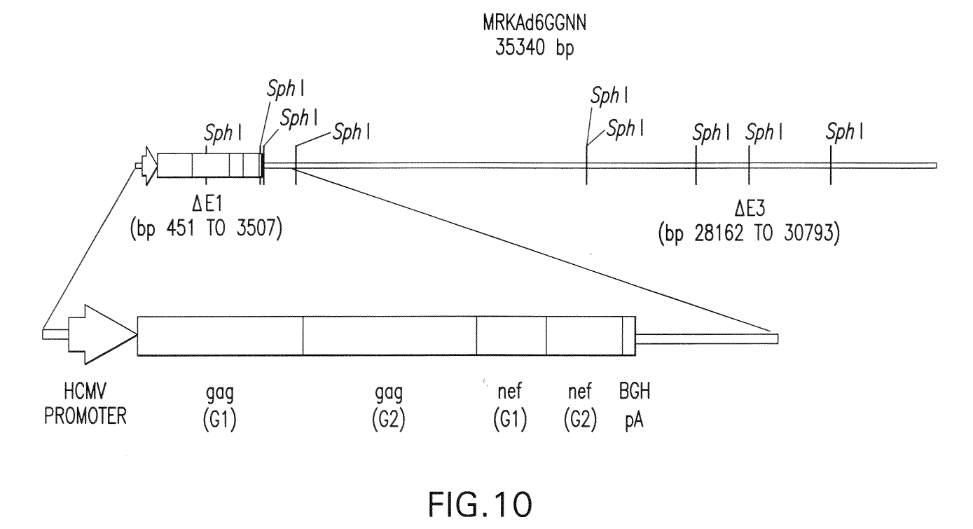

MRKAd6GGNN is depicted in FIG. 10. The vector is a modification of a prototype Group C Ad6 whose genetic sequence was determined at Merck. The E1 region of the wild-type Ad6 (nt 451-3507) is deleted and replaced with the transgene. The transgene contains the gag-gag-nef-nef expression cassette consisting of: 1) the immediate early gene promoter from the human cytomegalovirus, 2) the coding sequence of the human immunodeficiency virus type 1 (HIV-1) gag global 1 gene fused to gag global 2, fused to nef global 1, fused to nef global 2 (amino acid sequence provided as SEQ ID NO: 94; an encoding nucleic acid sequence provided as SEQ ID NO: 43), and 3) the bovine growth hormone polyadenylation signal sequence. The amino acid sequence of the gaggagnefnef protein was generated from Example 1. Codons were selected to optimize expression in human cells and to reduce regions of homology within the coding sequences. No more than 12 consecutive by are homologous between the two gag or two nef coding sequences. The gag open reading frames encode the matrix, capsid, and nucleocapsid proteins. The nef open reading frames were altered by mutating the myristoylation site located at Gly-2 to an alanine. This mutation prevents attachment of nef to the cytoplasmic membrane and retrotrafficking into endosomes, thereby functionally inactivating nef. In addition to the deletion of the E1 region, the vector has an E3 deletion (nt 28162 to 30793) in order to accommodate the transgene.

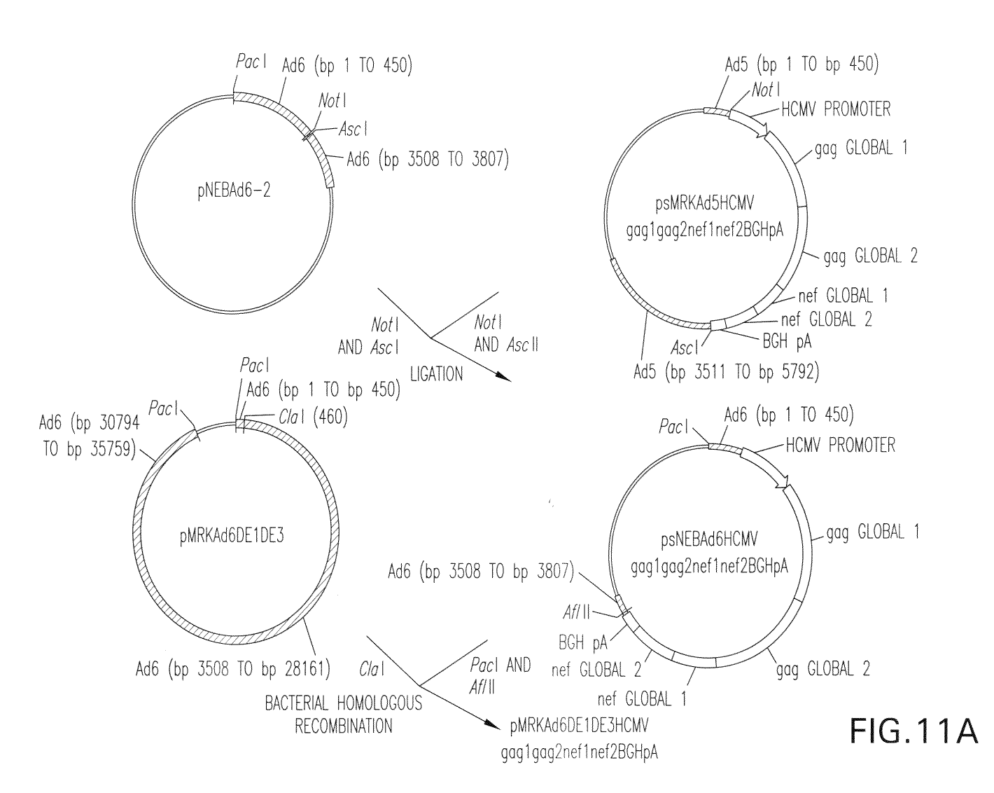

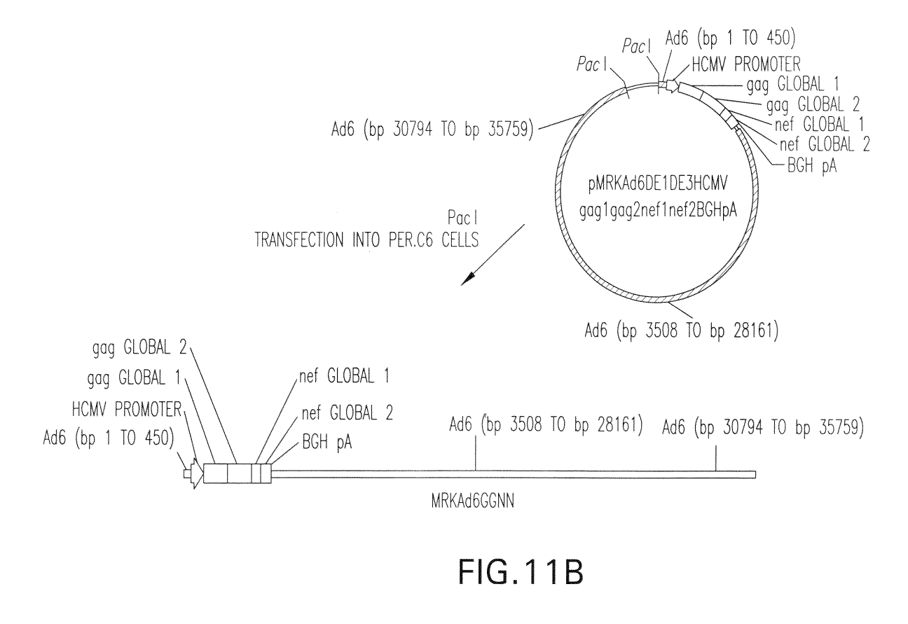

Key steps involved in the construction of MRKAd6GGNN are depicted in FIGS. 11A-B and described in the text that follows.

### (1) Construction of Adenoviral Shuttle Vector:

The shuttle plasmid psNEBAd6HCMVgag1gag2nef1nef2BGHpA was constructed by transferring the gaggagnefnef transgene from Ad5 shuttle plasmid psMRKAd5DE1gag1gag2nef1nef2BGHpA (described in Example 4) into the AscI and NotI sites in pNEBAd6-2. To obtain the gaggagnefnef transgene fragment, psMRKAd5DE1gag1gag2nef1nef2BGHpA was digested with NotI and AscI and the desired fragment gel purified. Once purified the NotI/AscI transgene fragment was ligated with pNEBAd6-2 also digested with Not I and AscI, generating psNEBAd6HCMVgag1gag2nef1nef2BGHpA. The genetic structure of psNEBAd6HCMVgag1gag2nef1nef2BGHpA was verified by restriction enzyme analysis and sequencing.

### (2) Construction of Pre-Adenovirus Plasmid:

To construct pre-adenovirus pMRKAd6DE1DE3HCMVgag1gag2nef1nef2BGHpA, the transgene containing fragment was liberated from shuttle plasmid psNEBAd6HCMVgag1gag2nef1nef2BGHpA by digestion with restriction enzymes PacI and AflII and gel purified. The purified transgene fragment was then co-transformed into E. coli strain BJ5183 with linearized (ClaI-digested) adenoviral backbone plasmid, pMRKAd6DE1DE3. Plasmid DNA isolated from BJ5183 transformants was then transformed into competent E. coli XL-1 Blue for screening by restriction analysis. The desired plasmid pMRKAd6DE1DE3HCMVgag1gag2nef1nef2BGHpA was verified by restriction enzyme digestion and DNA sequence analysis.

### (3) Generation of Recombinant MRKAd6GGNN:

To prepare virus the pre-adenovirus plasmid pMRKAd6DE1DE3HCMVgag1gag2nef1nef2BGHpA was rescued as infectious virions in PER.C6® adherent monolayer cell culture. To rescue infectious virus, 10 μg of pMRKAd6DE1DE3gag1gag2nef1nef2BGHpA was digested with restriction enzyme PacI (New England Biolabs) and then transfected into one T25 flask of PER.C6® cells using the calcium phosphate co-precipitation technique. PacI digestion releases the viral genome from plasmid sequences, allowing viral replication to occur after entry into PER.C6® cells. Infected cells and media were harvested 10 days post-transfection, after complete viral cytopathic effect (CPE) was observed. The virus stock was amplified by 2 passages in PER.C6® cells. At passage 2, virus was purified on CsCl density gradients. To verify that the rescued virus had the correct genetic structure, viral DNA was isolated and analyzed by restriction enzyme (SphI and BglII) analysis. The rescued virus was referred to as MRKAd6GGNN (also called MRKAd6DE1DE3HCMVgag1gag2nef1nef2BGHpA).

### Example 5

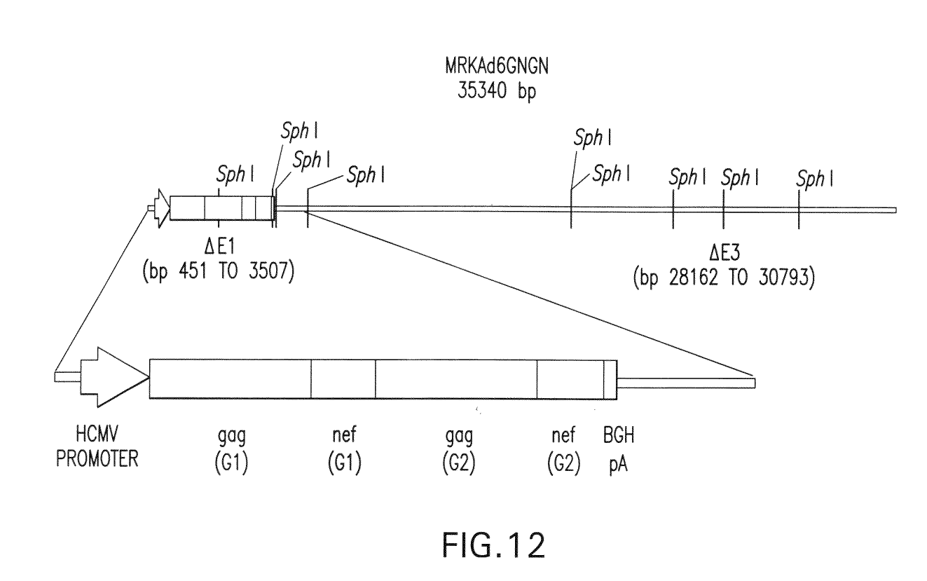

Construction of an Ad6 Vector Containing an HIV-1 gag-nef-gag-nef Fusion Transgene MRKAd6GNGN is depicted in FIG. 12. The vector is a modification of a prototype Group C Ad6 whose genetic sequence was determined at Merck. The E1 region of the wild-type Ad6 (nt 451-3507) is deleted and replaced with the transgene. The transgene contains the gag-nef-gag-nef expression cassette consisting of: 1) the immediate early gene promoter from the human cytomegalovirus, 2) the coding sequence of the human immunodeficiency virus type 1 (HIV-1) gag global 1 gene fused to nef global 1, fused to gag global 2, fused to nef global 2 (amino acid sequence provided as SEQ ID NO: 96; an encoding nucleic acid sequence provided as SEQ ID NO: 44), and 3) the bovine growth hormone polyadenylation signal sequence. The amino acid sequence of the gagnefgagnef protein was generated from Example 1. Codons were selected to optimize expression in human cells and to reduce regions of homology within the coding sequences. No more than 12 consecutive by are homologous between the two gag or two nef coding sequences. The gag open reading frames encode the matrix, capsid, and nucleocapsid proteins. The nef open reading frames were altered by mutating the myristoylation site located at Gly-2 to an alanine. This mutation prevents attachment of nef to the cytoplasmic membrane and retrotrafficking into endosomes, thereby functionally inactivating nef. In addition to the deletion of the E1 region, the vector has an E3 deletion (nt 28162 to 30793) in order to accommodate the transgene.

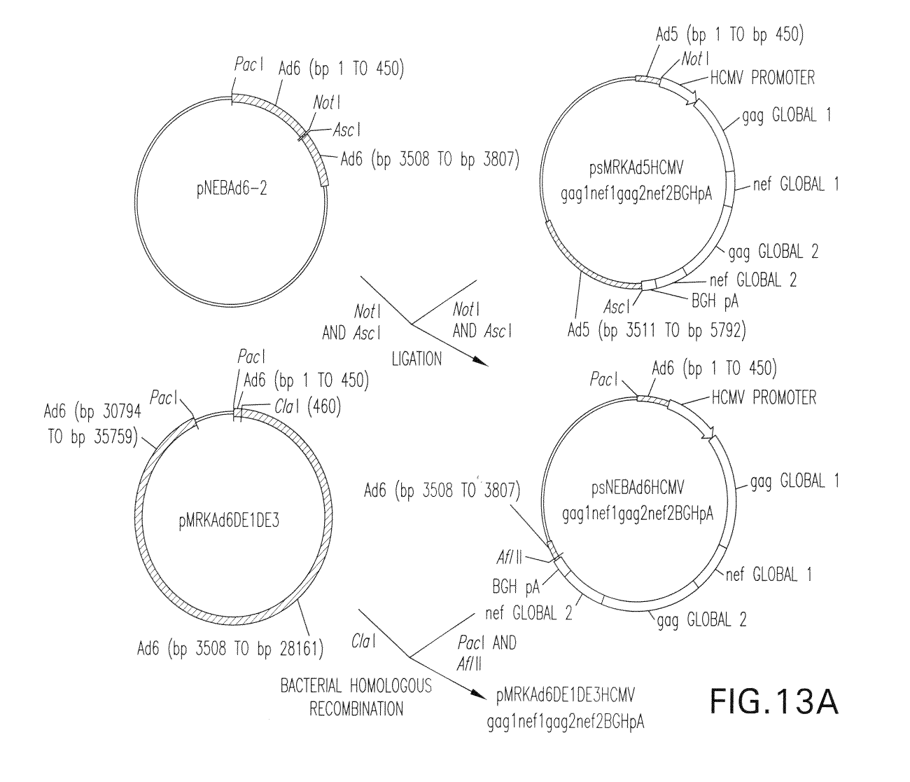

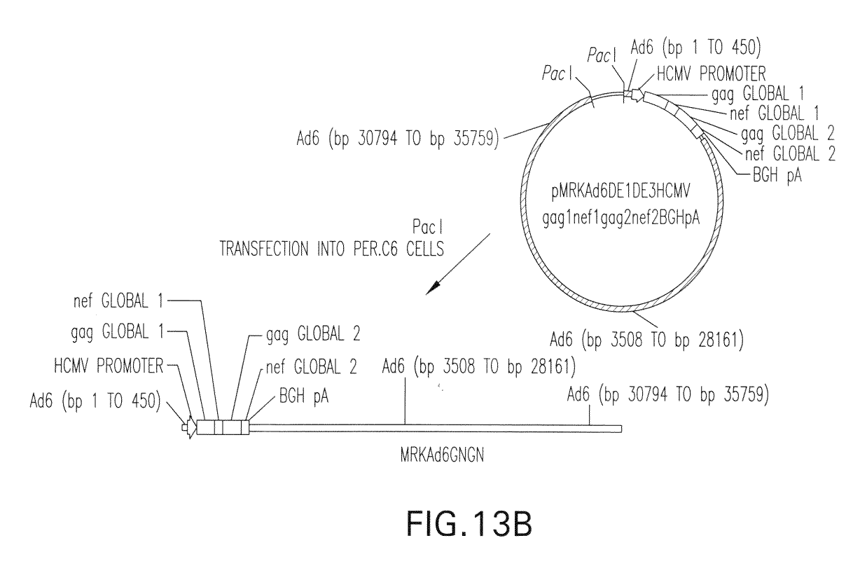

Key steps involved in the construction of MRKAd6GNGN are depicted in FIGS. 13 A-B and described in the text that follows.

### (1) Construction of Adenoviral Shuttle Vector:

The shuttle plasmid psNEBAd6HCMVgag1nef1gag2nef2BGHpA was constructed by transferring the gagnefgagnef transgene from Ad5 shuttle plasmid psMRKAd5HCMVgag1nef1gag2nef2BGHpA (described in Example 5) into the AscI and NotI sites in pNEBAd6-2. To obtain the gagnefgagnef transgene fragment, psMRKAd5HCMVgag1nef1gag2nef2BGHpA was digested with NotI and AscI and the desired fragment gel purified. Once purified the NotI/AscI transgene fragment was ligated with pNEBAd6-2 also digested with Not I and AscI, generating psNEBAd6HCMVgag1nef1gag2nef2BGHpA. The genetic structure of psNEBAd6HCMVgag1nef1gag2nef2BGHpA was verified by restriction enzyme analysis and sequencing.

(2) Construction of Pre-Adenovirus Plasmid:

To construct pre-adenovirus pMRKAd6DE1DE3HCMVgag1nef1gag2nef2BGHpA, the transgene containing fragment was liberated from shuttle plasmid psNEBAd6HCMVgag1nef1gag2nef2BGHpA by digestion with restriction enzymes PacI and AflI and gel purified. The purified transgene fragment was then co-transformed into E. coli strain BJ5183 with linearized (ClaI-digested) adenoviral backbone plasmid, pMRKAd6DE1DE3. Plasmid DNA isolated from BJ5183 transformants was then transformed into competent E. coli XL-1 Blue for screening by restriction analysis. The desired plasmid pMRKAd6DE1DE3HCMVgag1nef1gag2nef2BGHpA was verified by restriction enzyme digestion and DNA sequence analysis.

### (3) Generation of Recombinant MRKAd6GNGN:

To prepare virus the pre-adenovirus plasmid pMRKAd6DE1DE3HCMVgag1nef1gag2nef2BGHpA was rescued as infectious virions in PER.C6® adherent monolayer cell culture. To rescue infectious virus, 10 μg of pMRKAd6DE1DE3HCMVgag1nef1gag2nef2BGHpA was digested with restriction enzyme PacI (New England Biolabs) and then transfected into one T25 flask of PER.C6® cells using the calcium phosphate co-precipitation technique. PacI digestion releases the viral genome from plasmid sequences, allowing viral replication to occur after entry into PER.C6® cells. Infected cells and media were harvested 10 days post-transfection, after complete viral cytopathic effect (CPE) was observed. The virus stock was amplified by 2 passages in PER.C6® cells. At passage 2, virus was purified on CsCl density gradients. To verify that the rescued virus had the correct genetic structure, viral DNA was isolated and analyzed by restriction enzyme (SphI and BglII) analysis. The rescued virus was referred to as MRKAd6GNGN (also called MRKAd6DE 1 DE3 HCMVgag1 nef1gag2nef2BGHpA).

### Example 6

### Construction of an Ad5 Vector Containing an HIV-1 Gag-Nef-Nef-Nef Fusion Transgene

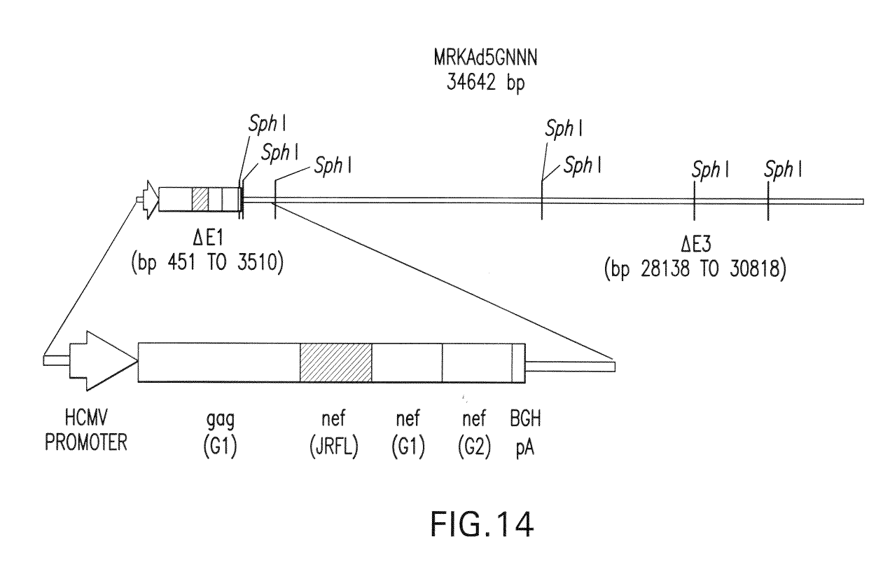

MRKAd5GNNN is depicted in FIG. 14. The vector is a modification of a prototype Group C Ad5 whose genetic sequence has been reported previously. The E1 region of the wild-type Ad5 (nt 451-3510) is deleted and replaced with the transgene. The transgene contains the gag-nef-nef-nef expression cassette consisting of: 1) the immediate early gene promoter from the human cytomegalovirus, 2) the coding sequence of the human immunodeficiency virus type 1 (HIV-1) gag global 1 gene fused to the coding sequence of the human immunodeficiency virus type 1 (HIV-1) nef (strain JRFL) gene, fused to nef global 1, fused to nef global 2 (amino acid sequence provided as SEQ ID NO: 95; an encoding nucleic acid sequence provided as SEQ ID NO: 45), and 3) the bovine growth hormone polyadenylation signal sequence. The amino acid sequence of the gag global 1 and nef global 1 and 2 proteins was generated from Example 1. The amino acid sequence of strain JRFL nef closely resembles the Clade B consensus amino acid sequence. Codons were selected to optimize expression in human cells and to reduce regions of homology within the coding sequences. No more than 12 consecutive by are homologous between the three nef coding sequences. The gag open reading frame encodes the matrix, capsid, and nucleocapsid proteins. The nef open reading frames were altered by mutating the myristoylation site located at Gly-2 to an alanine. This mutation prevents attachment of nef to the cytoplasmic membrane and retrotrafficking into endosomes, thereby functionally inactivating nef. In addition to the deletion of the E1 region, the vector has an E3 deletion (nt 28138 to 30818) in order to accommodate the transgene.

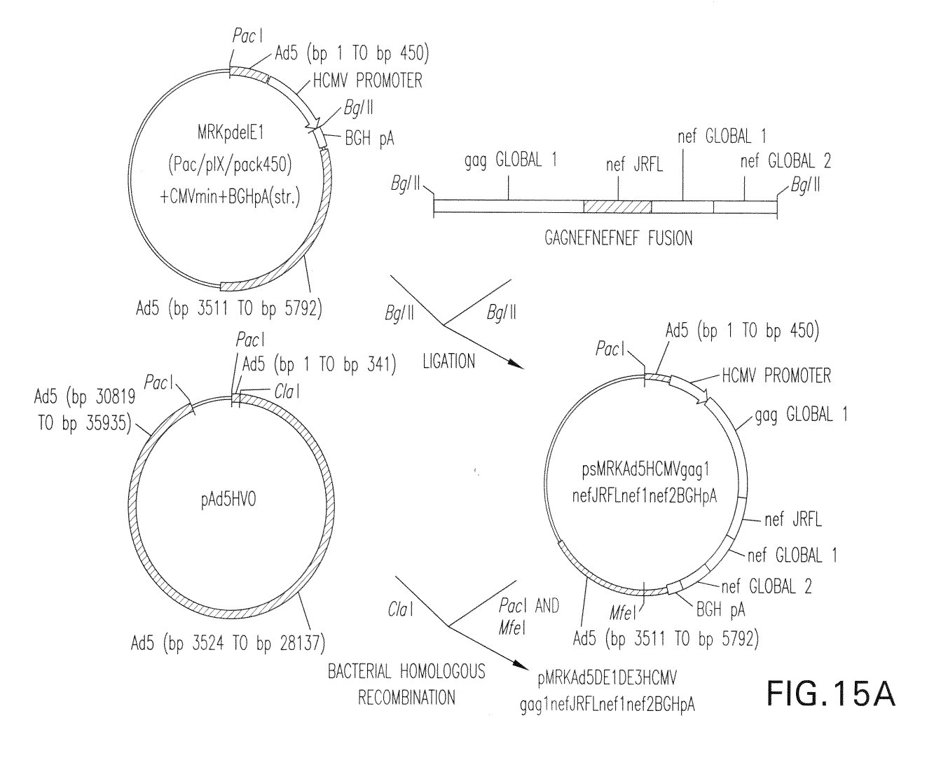

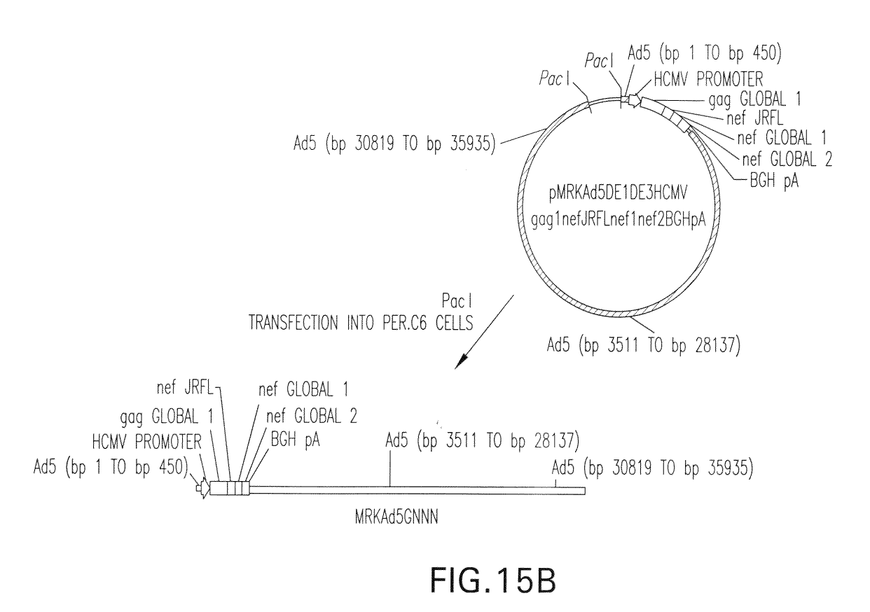

Key steps involved in the construction of MRKAd5GNNN are depicted in FIGS. 15 A-B and described in the text that follows.

(1) Construction of Adenoviral Shuttle Vector:

The shuttle plasmid psMRKAd5HCMVgag1nefJRFLnef1nef2BGHpA was constructed by inserting a synthetic full-length codon-optimized HIV-1 gagnefnefnef fusion gene into MRKpdelE1 (Pac/pIX/pack450)+CMVmin+BGHpA (str.). The synthetic full-length codon-optimized HIV-1 gagnefnefnef gene was synthesized at DNA2.0. The synthesized gene was ligated into the BglII restriction endonuclease site in MRKpdelE1 (Pac/pIX/pack450)+CMVmin+BGHpA (str.), generating plasmid psMRKAd5HCMVgag1nefJRFLnef1nef2BGHpA. The genetic structure of psMRKAd5HCMVgag1nefJRFLnef1nef2BGHpA was verified by restriction enzyme and DNA sequence analyses.

### (2) Construction of Pre-Adenovirus Plasmid:

To construct pre-adenovirus pMRKAd5DE1DE3HCMVgag1nefJRFLnef1nef2BGHpA, the transgene containing fragment was liberated from shuttle plasmid psMRKAd5HCMVgag1nefJRFLnef1nef2BGHpA by digestion with restriction enzymes PacI and MfeI and gel purified. The purified transgene fragment was then co-transformed into E. coli strain BJ5183 with linearized (ClaI-digested) adenoviral backbone plasmid, pAd5HVO (also referred to as pAd5 E1-E3-). Plasmid DNA isolated from BJ5183 transformants was then transformed into competent E. coli XL-1 Blue for screening by restriction analysis. The desired plasmid pMRKAd5DE1DE3HCMVgag1nefJRFLnef1nef2BGHpA was verified by restriction enzyme digestion and DNA sequence analysis.

### (3) Generation of Recombinant MRKAd5GNNN:

To prepare virus the pre-adenovirus plasmid pMRKAd5DE1DE3HCMVgag1nefJRFLnef1nef2BGHpA was rescued as infectious virions in PER.C6® adherent monolayer cell culture. To rescue infectious virus, 10 μg of pMRKAd5DE1DE3HCMVgag1nefJRFLnef1nef2BGHpA was digested with restriction enzyme PacI (New England Biolabs) and then transfected into one T25 flask of PER.C6® cells using the calcium phosphate co-precipitation technique. PacI digestion releases the viral genome from plasmid sequences, allowing viral replication to occur after entry into PER.C6® cells. Infected cells and media were harvested 10 days post-transfection, after complete viral cytopathic effect (CPE) was observed. The virus stock was amplified by 2 passages in PER.C6® cells. At passage 2, virus was purified on CsCl density gradients. To verify that the rescued virus had the correct genetic structure, viral DNA was isolated and analyzed by restriction enzyme (SphI and BglII) analysis. The rescued virus was referred to as MRKAd5GNNN (also called MRKAd5DE1DE3HCMVgag1nefJRFLnef1nef2BGHpA).

### Example 7

### Construction of an Ad6 Vector Containing an HIV-1 Gag-Nef-Nef-Nef Fusion Transgene

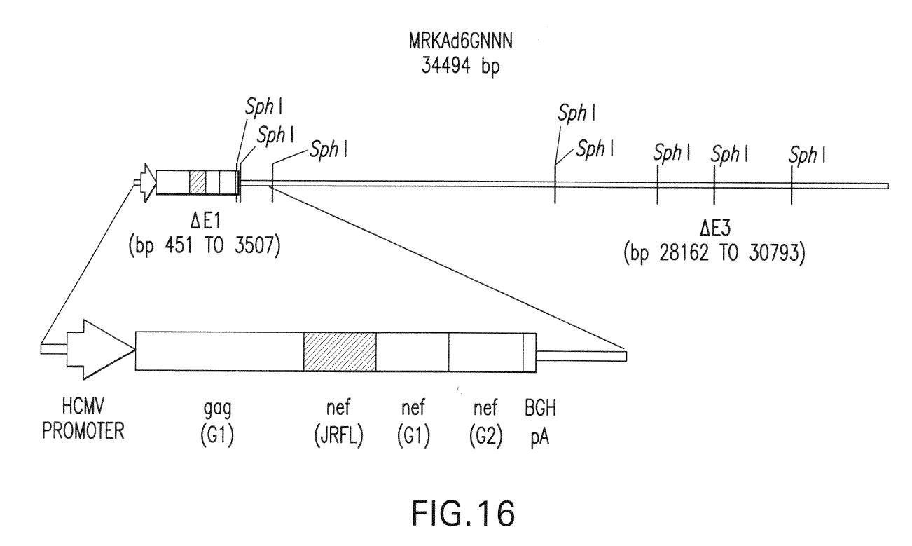

MRKAd6GNNN is depicted in FIG. 16. The vector is a modification of a prototype Group C Ad6 whose genetic sequence was determined at Merck. The E1 region of the wild-type Ad6 (nt 451-3507) is deleted and replaced with the transgene. The transgene contains the gag-nef-nef-nef expression cassette consisting of: 1) the immediate early gene promoter from the human cytomegalovirus, 2) the coding sequence of the human immunodeficiency virus type 1 (HIV-1) gag global 1 gene fused to the coding sequence of the human immunodeficiency virus type 1 (HIV-1) nef (strain JRFL) gene, fused to nef global 1, fused to nef global 2 (amino acid sequence provided as SEQ ID NO: 95; an encoding nucleic acid sequence provided as SEQ ID NO: 45), and 3) the bovine growth hormone polyadenylation signal sequence. The amino acid sequence of the gag global 1 and nef global 1 and 2 proteins was generated from Example 1. The amino acid sequence of strain JRFL nef closely resembles the Clade B consensus amino acid sequence. Codons were selected to optimize expression in human cells and to reduce regions of homology within the coding sequences. No more than 12 consecutive by are homologous between the three nef coding sequences. The gag open reading frame encodes the matrix, capsid, and nucleocapsid proteins. The nef open reading frames were altered by mutating the myristoylation site located at Gly-2 to an alanine. This mutation prevents attachment of nef to the cytoplasmic membrane and retrotrafficking into endosomes, thereby functionally inactivating nef. In addition to the deletion of the E1 region, the vector has an E3 deletion (nt 28162 to 30793) in order to accommodate the transgene.

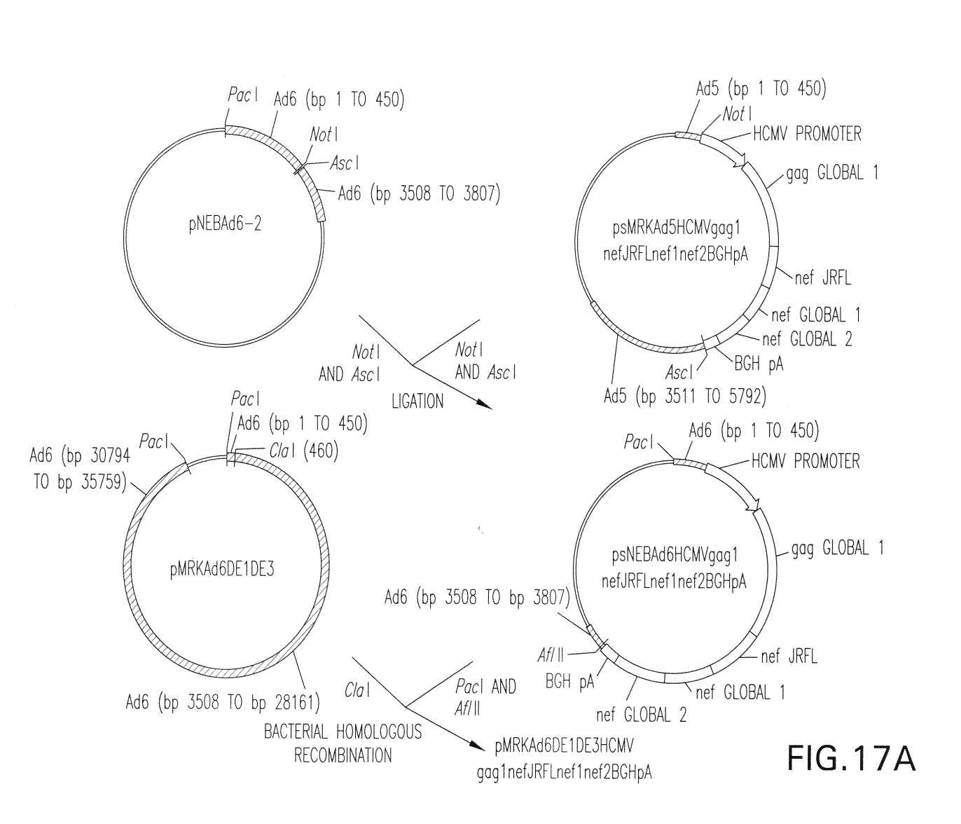

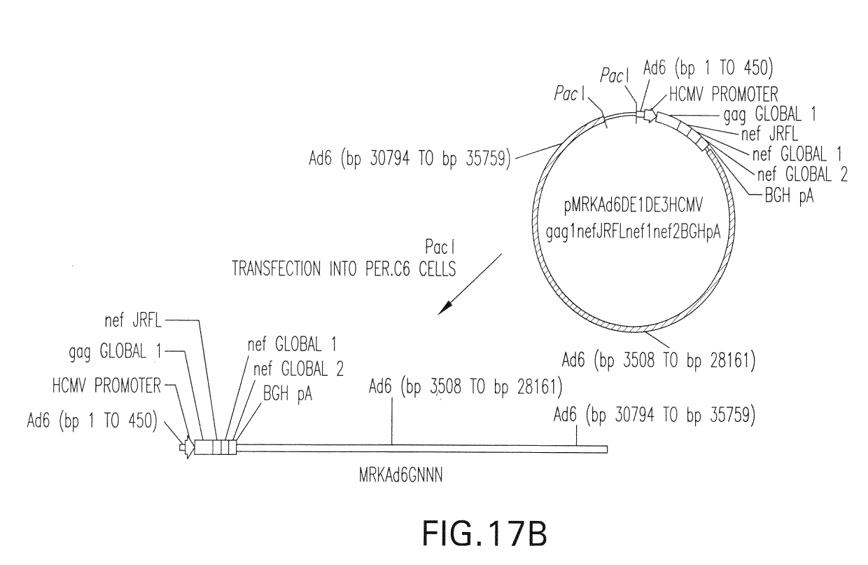

Key steps involved in the construction of MRKAd6GNNN are depicted in FIGS. 17 A-B and described in the text that follows.

(1) Construction of Adenoviral Shuttle Vector:

The shuttle plasmid psNEBAd6HCMVgag1nefJRFLnef1nef2BGHpA was constructed by transferring the gagnefnefnef transgene from Ad5 shuttle plasmid psMRKAd5DE1HCMVgag1nefJRFLnef1nef2BGHpA (described in Example 8) into the AscI and NotI sites in pNEBAd6-2. To obtain the gagnefnefnef transgene fragment, psMRKAd5DE1HCMVgag1nefJRFLnef1nef2BGHpA was digested with NotI and AscI and the desired fragment gel purified. Once purified the NotI/AscI transgene fragment was ligated with pNEBAd6-2 also digested with Not I and AscI, generating psNEBAd6HCMVgag1nefJRFLnef1nef2BGHpA. The genetic structure of psNEBAd6HCMVgag1nefJRFLnef1nef2BGHpA was verified by restriction enzyme analysis and sequencing.

### (2) Construction of Pre-Adenovirus Plasmid:

To construct pre-adenovirus pMRKAd6DE1DE3HCMVgag1nefJRFLnef1nef2BGHpA, the transgene containing fragment was liberated from shuttle plasmid psNEBAd6HCMVgag1nefJRFLnef1nef2BGHpA by digestion with restriction enzymes PacI and AflI and gel purified. The purified transgene fragment was then co-transformed into E. coli strain BJ5183 with linearized (ClaI-digested) adenoviral backbone plasmid, pMRKAd6DE1DE3. Plasmid DNA isolated from BJ5183 transformants was then transformed into competent E. coli XL-1 Blue for screening by restriction analysis. The desired plasmid pMRKAd6DE1DE3HCMVgag1nefJRFLnef1nef2BGHpA was verified by restriction enzyme digestion and DNA sequence analysis.

### (3) Generation of Recombinant MRKAd6GNGN:

To prepare virus the pre-adenovirus plasmid pMRKAd6DE1DE3HCMVgag1nefJRFLnef1nef2BGHpA was rescued as infectious virions in PER.C6® adherent monolayer cell culture. To rescue infectious virus, 10 μg of pMRKAd6DE1DE3HCMVgag1nefJRFLnef1nef2BGHpA was digested with restriction enzyme PacI (New England Biolabs) and then transfected into one T25 flask of PER.C6® cells using the calcium phosphate co-precipitation technique. PacI digestion releases the viral genome from plasmid sequences, allowing viral replication to occur after entry into PER.C6® cells. Infected cells and media were harvested 10 days post-transfection, after complete viral cytopathic effect (CPE) was observed. The virus stock was amplified by 2 passages in PER.C6® cells. At passage 2, virus was purified on CsCl density gradients. To verify that the rescued virus had the correct genetic structure, viral DNA was isolated and analyzed by restriction enzyme (SphI and BglII) analysis. The rescued virus was referred to as MRKAd6GNNN (also called MRKAd6DE1DE3HCMVgag1nefJRFLnef1nef2BGHpA).

### Example 8

### In Vitro Gene Expression

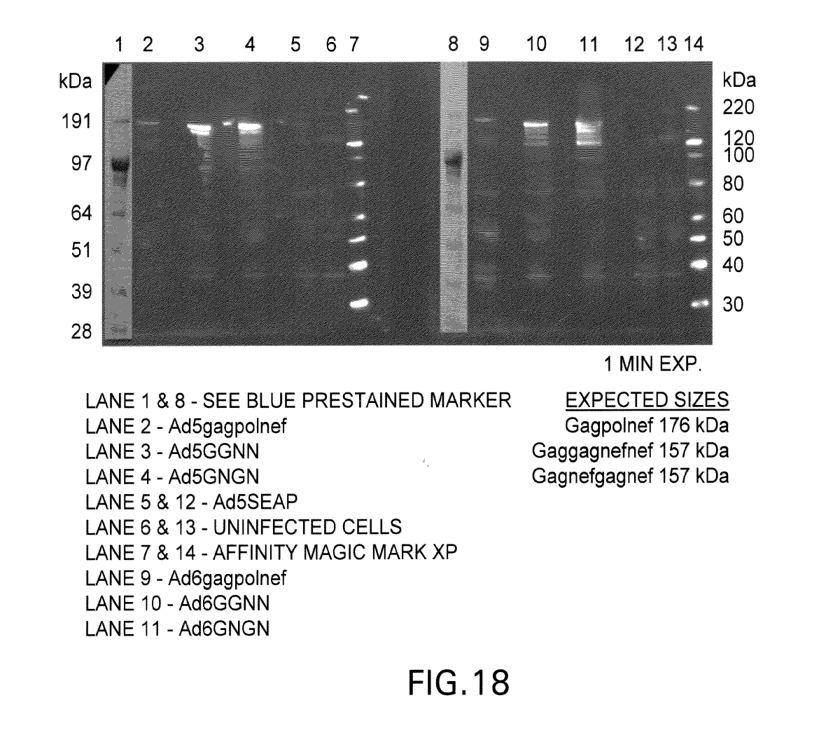

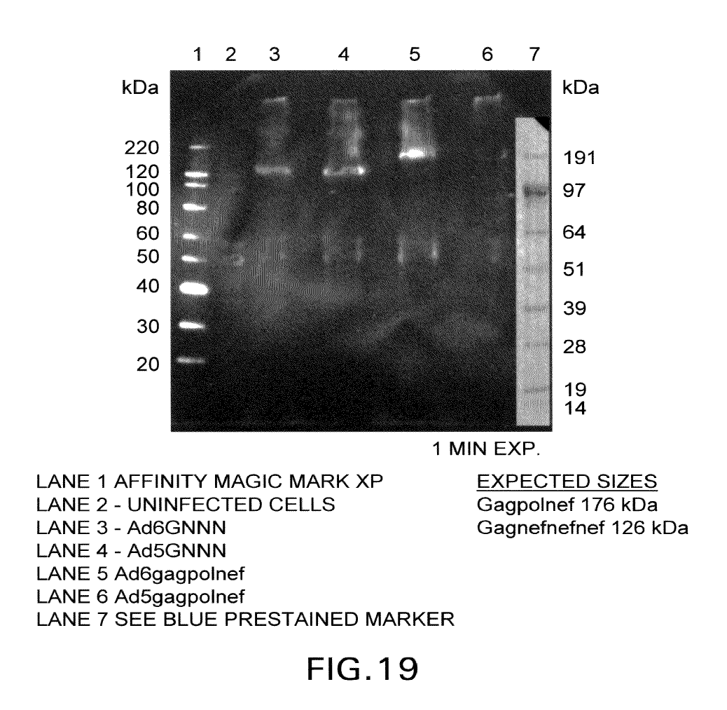

Western blots (FIG. 18 and FIG. 19) were performed to demonstrate that infection of cells with the six recombinant Ad vectors (MRKAd5GGNN, MRKAd5GNGN, MRKAd5GNNN, MRKAd6GGNN, MRKAd6GNGN and MRKAd6GNNN) resulted in the expression of the desired fusion proteins. As positive controls, similar Ad5 and Ad6 constructs expressing a clade B gagpolnef fusion were used and as a negative control an Ad5 vector expressing secretory alkaline phosphatase was used. For the assays, monolayers of PER.C6® cells in T-25 flasks were infected with the vectors independently at a multiplicity of infection of 100 viral particles per cell and incubated for approximately 72 hours. Infected cells and media were collected and the cells pelleted by centrifugation. Cell pellets were then resuspended in 0.5 ml of media and mixed with 0.5 ml of 1.66× lysis buffer (249 mM NaCl+83 mMTris-HCL+0.83% NP-40+0.83% DOC+Roche Protease Inhibitors (cat #1697498)). Samples of the cell lysates (20 μl) were then separated by SDS-polyacrylamide gel electrophoresis (PAGE) on 4-12% acrylamide gels and blotted to PVDF membranes. The fusion proteins were detected using a mouse monoclonal Ab to HIV-1 gag p24 (Advanced Biotechnologies cat# 13-102-100, at a 1:1000 dilution) as the primary antibody and an HRP conjugated F(ab′)₂ goat anti mouse IgG Fcγ as the secondary antibody (Jackson ImmunoResearch cat# 115-036-008, at 1:5000 dilution). Fusion proteins of the predicted molecular weight were seen for each vector (157 kDa for gaggagnefnef and gagnefgagnef; 126 kDa for gagnefnefnef; 176 kDa for gagpolnef).

### Example 9

### Immunizations

Rhesus macaques were between 3.4-12.0 kg in mass. In all cases, the total dose of each vaccine was suspended in 1 mL of buffer at a concentration of 1.0×10¹⁰ viral particles/mL. The macaques were anesthetized (ketamine) and the vaccines delivered intramuscularly in 0.5 mL aliquots into both deltoid muscles using tuberculin syringes (Becton-Dickinson, Franklin Lakes, N.J.). Immunizations occurred on weeks 0, 4, and 24. Peripheral blood mononuclear cells (PBMCs) were prepared from blood samples collected at several time points during the immunization regimen. All animal care and treatment was in accordance with the standards approved by the Institutional Animal Care and Use Committee according to the principles set forth in the Guide for Care and Use of Laboratory Animals, Institute of Laboratory Animal Resources, National Research Council.

### Example 10

Antibody Titers Against HIV-1 gag and HIV-1 nef Elicted by Vaccine Constructs

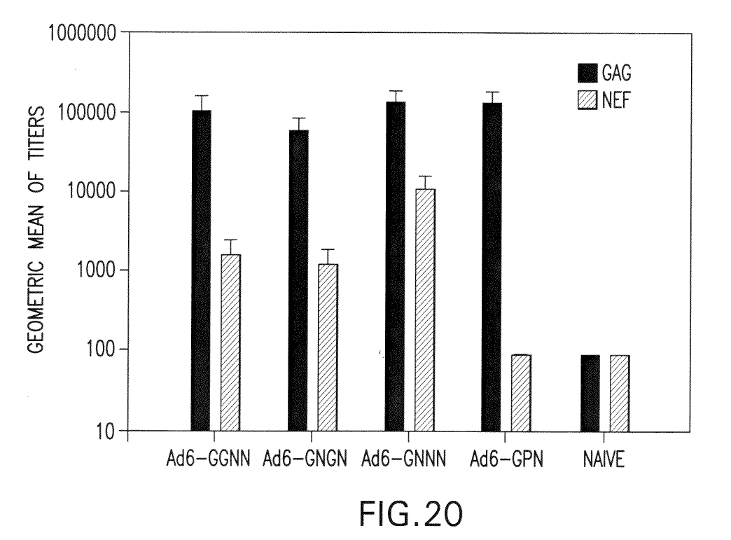

Groups of five (5) mice were immunized with the adenovector vaccine constructs: Ad6 Vector containing an HIV-1 gag-gag-nef-nef (Example 4; Ad6-GGNN), Ad6 Vector containing an HIV-1 gag-nef-gag-nef fusion transgene (Example 5; Ad6-GNGN), Ad6 Vector containing an HIV-1 gag-nef-nef-nef fusion transgene (Example 7; Ad6-GNNN), Ad6 Vector containing an HIV-1 gag-pol-nef fusion transgene (Ad6-GPN; see, International Publication Number WO 2006/020480, published Feb. 23, 2006), and a naïve control group. Sera were collected from each mouse and endpoint titers vs. HIV-1 Gag and HIV-1 Nef proteins were determined by ELISA. The geometric mean of each group is shown in FIG. 20. Error bars show the standard error of the geometric mean. Gag responses to all vaccines are high; vaccines encoding multiple versions of nef as described in this Invention have higher titers than the single-version Ad6-GPN.

### Example 11

### Elispot Responses in Rhesus Macaques Elicted by Vaccine Constructs

Groups of five (5) Rhesus Macaques were immunized with adenovector vaccine constructs: Ad6 Vector containing an HIV-1 gag-gag-nef-nef (Example 4; Ad6-GGNN); Ad6 Vector containing an HIV-1 gag-nef-gag-nef fusion transgene (Example 5; Ad6-GNGN); Ad6 Vector containing an HIV-1 gag-nef-nef-nef fusion transgene (Example 7; Ad6-GNNN); Ad6 Vector containing an HIV-1 gag-pol-nef fusion transgene, see International Publication No. WO 2006/020480, published Feb. 23, 2006; Ad6 Vector containing an HIV-1 gag-pol fusion transgene, see International Publication No. WO 2006/020480, published Feb. 23, 2006; and trivalent combination of an Ad6 Vector containing an HIV-1 gag transgene, an Ad6 Vector containing an HIV-1 pol transgene, and an Ad6 Vector containing an HIV-1 nef transgene; see International Publication No. WO 2006/020480, published Feb. 23, 2006.

Ninety-six-well flat-bottomed plates (Millipore, Immobilon-P membrane) were coated with 1 μg/well of anti-gamma interferon (IFN-γ) mAb MD-1 (U-Cytech-BV) in sterile PBS (phosphate buffered saline) overnight at 4° C. The plates were washed three times with PBS and blocked with complete R10 medium (RPMI 1640 plus 10% fetal bovine serum) for 2 hours at 37° C. The medium was decanted from the plates and freshly isolated peripheral blood mononuclear cells (PBMC) were added at 2-4×10⁵ cells/well in R10. Pools of synthetic peptides (15 amino acids in length overlapping by 11 amino acids; Synpep, CA) were diluted in R10 and added to the wells in duplicate at a final concentration of 2-3 μg/ml. Peptide sequences were based on isolates or consensuses of HIV-1 clades A, B and C. The assigned labels in the following Table 3 are either common names or GenBank accession numbers and will be readily appreciated by the skilled artisans:

TABLE 3

| PROTEIN | CLADE B | CLADE A | CLADE C |
|---|---|---|---|
| Gag | CAM-1 | 90CF4071 | SEQ ID NO: 91 |
| Nef | JRFL | SE8891 | IN21068 |
| Pol | HXB2 | | |

Pol peptides were divided into two pools that approximately bisect the Pol protein, due to the large number of peptides that span the Pol protein. These were labeled Pol-1B and Pol-2B. “Mock” control wells (no peptide added) and positive control wells (Staphylococcus enterotoxin B, SEB; Sigma) were included for each sample. Assay plates were incubated for 20-24 hours at 37° C. in 5% CO₂. Plates were washed six times with PBST (PBS, 0.05% Tween 20™) and 100 μl/well of a 1:400 dilution of biotinylated anti-IFN-γ polyclonal antibody (U-Cytech-BV) was added. The plates were incubated overnight at 4° C. and then washed 4 times with PBST. Streptavidin-alkaline phosphatase (SA-AP, BD Pharmingen) was diluted 1:2500 and added to each well at 100 μl/well. Plates were incubated 2 hours at room temperature and then washed 4 times with PBST. Spots were developed by incubating with 100 μl/well of NBT/BCIP (Pierce) for 7 minutes at room temperature and then washing 4 times with water. Plates were allowed to dry overnight on the benchtop and wells were imaged using an ELISpot imager system (AID, Germany). Spots, which represent IFN-γ secreting cells, were counted by the AID imager, averaged across duplicate wells, and normalized to number of spots per 1×10⁶ PBMC for each antigen. For an ELISpot response to be considered as positive, the number of spot forming cells must be greater than or equal to 55 spots/10⁶ PBMCs and greater than or equal to 4-fold the media-only negative control wells. These stringent criteria exclude greater than 99% of false positives.

The disclosed antigen sequences increase non-clade B responses to GagA and GagC relative to Ad6gagpolnef, Ad6gagpol, and Ad6gag+Ad6pol+Ad6nef; see Table 4 and Table 5. In conjunction with existing clade B antigen sequences, these antigen sequences can be expected to increase breadth of response to non-clade B HIV-1 isolates.

In another experiment, adenovector vaccine constructs were synthesized as follows: Ad6 vector containing the gag N16.1 transgene (SEQ ID NO: 1), Ad6 vector containing the nef N16.1 transgene (SEQ ID NO: 3), Ad6 vector the containing the nef N16.2 transgene (SEQ ID NO: 4). A group of four (4) rhesus macaques (“Group 2”) was immunized with these constructs plus Ad6gagpol and Ad6nef; another group of four (4) rhesus macaques (“Group 1”) was immunized with Ad6gag+Ad6pol+Ad6nef. Immunizations followed the description in Example 9. At week 28, four (4) weeks after the boosting injection, responses were mapped by ELISpot to regions of the proteins listed in Table 3 spanning 30 amino acids. Results were as follows. For GagA, Group 1 had 3/4 responders (mean number of regions per individual, 1.0) vs. Group 2 (4/4, mean 2.75). For GagC, both groups had 4/4 responders, but Group 1 had a mean of 2.0 vs. Group 2 with a mean of 2.5. For NefA, Group 1 had 2/4 responders (mean 0.75), and Group 2 had 3/4 responders (mean 1.0). For NefC, Group 1 had 1/4 responders (mean 0.5), and Group 2 had 2/4 responders (mean 1.25). In each case, the breadth of response to clade A and clade C antigens was increased in Group 2 over Group 1.

TABLE 4
ELISPOT RESPONSES TO VACCINE CONSTRUCTS
IN RHESUS MACAQUES IN SPOT FORMING CELLS
PER MILLION (10)6 PERIPHERAL BLOOD MONOCYTES,
4 WEEKS AFTER PRIMING INJECTION
WEEK 4 ELISPOT GEOMEAN (% Responders Based
On ÷55 Spots/10⁶ Cells And ÷4x Mock)

| Vaccine | GagB | GagC | GagA | NefB | NefC | NefA | Pol-1B | Pol-2B |
|---|---|---|---|---|---|---|---|---|
| Ad6gagpolnef* | 260 | 145 | 156 | 58 | 12 | 13 | 159 | 609 |
| | (100%) (60%) | (80%) | (40%) | (20%) | (20%) | (80%) | (100%) | |
| Ad6gaggagnefnef | 844 | 794 | 725 | 45 | 41 | 78 | 3 | 5 |
| Ad6-SEQ ID NO: 94 | (100%) | (100%) | (100%) | (60%) | (40%) | (60%) | (0%) | (0%) |
| Ad6gagnefgagnef | 796 | 893 | 649 | 36 | 36 | 51 | 12 | 19 |
| Ad6-SEQ ID NO: 96 | (100%) | (100%) | (100%) | (20%) | (40%) | (40%) | (0%) | (0%) |
| Ad6gagpol* | 283 | 212 | 156 | 4 | 3 | 2 | 356 | 291 |
| | (80%) | (80%) | (80%) | (0%) | (0%) | (0%) | (100%) | (80%) |
| Ad6gagnefnefnef | 281 | 397 | 322 | 148 | 52 | 59 | 6 | 6 |
| Ad6-SEQ ID NO: 95 | (100%) | (100%) | (100%) | (80%) | (40%) (40%) | (0%) | (0%) | |
| Ad6gag + Ad6pol + | 335 | 162 | 176 | 262 | 43 | 46 | 70 | 86 |
| Ad6nef* | (100%) | (100%) | (100%) | (100%) | (40%) | (40%) | (60%) | (80%) |

*see International Publication No. WO 06/020480,? published FEB. 23, 2006

TABLE 5
ELISPOT RESPONSES TO VACCINE CONSTRUCTS
IN RHESUS MACAQUES IN SPOT FORMING CELLS
PER MILLION (10)6 PERIPHERAL BLOOD MONOCYTES,
4 WEEKS AFTER BOOSTING INJECTION (WEEK 28).
WEEK 28 ELISPOT GEOMEAN (% Responders Based
On ÷55 Spots/10⁶ Cells And ÷4x Mock)

| Vaccine | GagB | GagC | GagA | NefB | NefC | NefA | Pol-1B | Pol-2B |
|---|---|---|---|---|---|---|---|---|
| MRKAd6gagpolnef* | 136 | 85 | 108 | 25 | 14 | 10 | 84 | 259 |
| | (80%) | (40%) | (60%) | (40%) | (0%) | (0%) | (60%) | (80%) |
| Ad6gaggagnefnef | 520 | 591 | 377 | 30 | 25 | 45 | 5 | 4 |
| Ad6-SEQ ID NO: 94 | (100%) | (100%) | (100%) | (40%) | (0%) | (40%) | (0%) | (0%) |
| Ad6gagnefgagnef | 546 | 631 | 346 | 13 | 16 | 24 | 6 | 10 |
| Ad6-SEQ ID NO: 96 | (100%) | (100%) | (100%) | (0%) | (0%) | (0%) | (0%) | (0%) |
| Ad6gagpol* | 223 | 194 | 176 | 7 | 5 | 3 | 214 | 198 |
| | (80%) | (80%) | (80%) | (0%) | (0%) | (0%) | (80%) | (80%) |
| Ad6gagnefhefnef | 276 | 365 | 307 | 122 | 47 | 45 | 5 | 9 |
| Ad6-SEQ ID NO: 95 | (100%) | (100%) | (100%) | (60%) | (40%) | (40%) | (0%) | (0%) |
| Ad6gag + Ad6pol + | 454 | 276 | 319 | 306 | 49 | 72 | 72 | 89 |
| Ad6nef* | (100%) | (100%) | (100%) | (100%) | (40%) | (60%) | (60%) | (80%) |

*see International Publication No. WO 06/020480, published FEB. 23, 2006

### Example 12

### Rhesus Multi-Color Intracellular Cytokine Staining

PBMCs from the protocol described in Example 11 and collected at week 28 corresponding to Table 5, previously frozen in 90% FBS and 10% DMSO freezing media and stored in liquid nitrogen were slowly thawed in complete RPMI medium (RPMI 1640 medium, 2 mM L-glutamine, 5×10⁻⁵ M (β-mercaptoethanol, 5 mM HEPES, plus 25 μg of pyruvic acid, 100 U of penicillin, and 100 μg of streptomycin per mL (all cell culture reagents were from Invitrogen, Grand Island, N.Y.) supplemented with 10% FBS (HyClone, Logan Utah). Cells were washed and counted using trypan blue exclusion dye (Sigma) by hemacytometer. 1×10⁶ PBMCs were placed per well of a 96 U bottom plate in 200 μL of complete RPMI medium and rested at 37° C. humidified 5% CO₂ incubator for 4-6 hours. Cells were then stimulated with 1 μg/mL of each costimulatory antibody (anti-CD28 and anti-CD49d; BD, San Jose Calif.), 10 μg/mL of Brefeldin A (Sigma) and various 15 mer peptide pools. Peptides used in ELISpot assays were also used for intracellular cytokine staining. The final concentration of each peptide in the pool was 0.4 mg/mL, and the pool was added to a final concentration of 2 μg/mL to each sample. Cells were incubated overnight (15-16 hours) at 37° C. in a humidified 5% CO₂ incubator. 20 μL per well of 20 mM EDTA (mass/volume in 1×PBS) was added to each well for 15 minutes. Cells were mixed and centrifuged at 500 G for 5 minutes. Cells were washed with FACS buffer (PBS+1% FBS+0.01% NaA₃), and stained with surface staining antibodies, CD 8 APC-Cy7 (Sk1, BD), CD3 PerCPCy 5.5 (SP23-2, BD) for 25-30 minutes. Cells were washed twice with FACS buffer, supernatant was removed, and cells were permeabilized with BD Cytofix/Cytoperm™ solution for 20 minutes at room temperature. Cells were washed twice with BD Perm/Wash™ buffer and stained with intracellular antibodies II-2 APC (MQ1-17H12, BD), TNF PE-Cy7 (MAb11, BD), MIP1β-PE (D21-1351, BD Biosciences) and IFN-γ FITC (MD-1, Biosource) for 55-60 minutes. Cells were washed four times with BD Perm/Wash™ buffer and fixed with 1% formaldehyde. Samples were acquired the same day on an LSRII instrument with an HTS loader (BD, San Jose, Calif.). Approximately 300,000 total events were acquired and the data was analyzed using FlowJo Analysis Software (Tree Star, Inc.). An electronic gate was drawn around the lymphocyte population, followed by a gate around the viable cells as determined by the Invitrogen dye. Of these a CD3 versus CD8 plot was drawn to determine CD3+CD8+ (hereafter named CD8 cells in this Example) and CD3+CD8− (hereafter named CD4 cells in this Example). CD4 cells are identified in this manner because mature T cells (as determined by the CD3+ staining will either be of the subtype CD4 or CD8. Therefore, the cells that are CD3+CD8− are an accurate quantitation of the CD4 helper T cell population. For each T cell subset, CD4 and CD8, the cells were plotted as side scatter vs. each cytokine. A gate was drawn to exclude the cytokine-negative cells. The Boolean gate feature (FlowJo) was used to create all the combinations of cytokine populations. Each of these populations was normalized as events per 10⁶ lymphocytes for the reported final results.

Monkeys vaccinated with MRKAd6gagpolnef, Ad6gaggagnefnef, Ad6gagnefgagnef, and Ad6gagnefnefnef in the protocol detailed in Example 11 were analyzed. Responses to non-clade B peptide pools (GagA, GagC, NefA, NefC) were as follows. In the MRKAd6gagpolnef vaccine group, only one monkey had a positive response to any non-clade B peptide pool, and the response was monofunctional (positive for only one out of four potential cytokines IFN-γ, IL-2, MIP1β, and TNFα). In the Ad6gaggagnefnef vaccine group and the Ad6gagnefgagnef vaccine group, every monkey had trifunctional positive responses to one or more non-clade B peptide pools. In the Ad6gagnefnefnef vaccine group, four monkeys had trifunctional positive responses to one or more non-clade B peptide pools, and the remaining monkey had a monofunctional response to GagA and GagC peptide pools. Polyfunctional responses are an accepted measure for the quality of an immune response; the great the frequency of polyfunctional responses, the more likely that an immunological challenge (such as a viral infection) will be successfully resolved. The increase in frequency and polyfunctionality of the N-mer consensus vaccines Ad6gaggagnefnef, Ad6gagnefgagnef, and Ad6gagnefnefnef in comparison with the MRKAd6gagpolnef vaccine against non-clade B antigens indicates a potentially more effective immune response.

### Example 13

Anti-HIV Antibodies in Rhesus Macaques Elicted by Vaccine Constructs

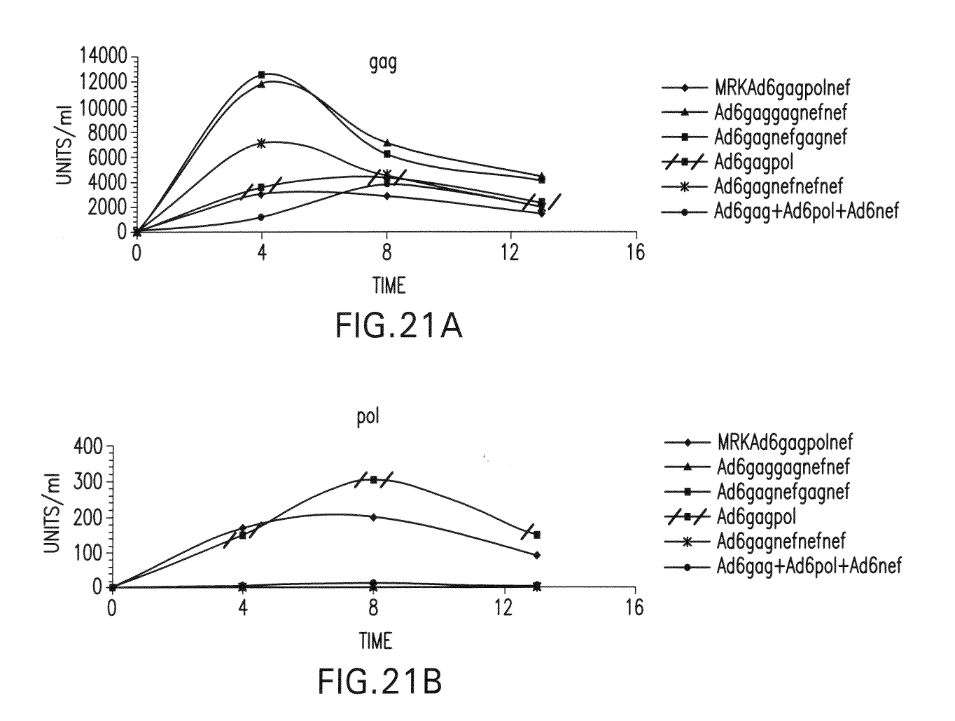

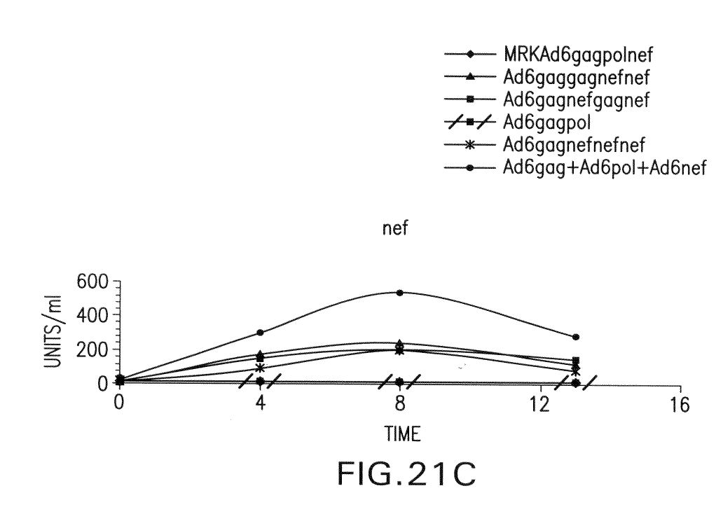

Sera from the vaccination protocol described in Example 11 were collected from rhesus macaques on the day of first immunization and at 4, 8, and 13 weeks post-immunization. Gag (HIV-1 p24, Protein Sciences, Meriden, Conn.), Pol (HIV-1 p66, Protein Sciences), and Nef (ImmunoDiagnostics, Woburn, Mass.) proteins were separately coupled to spectrally distinct carboxylated polymer LUMINEX™ microspheres (Luminex Corp., Austin, Tex.) via a mixture of EDC (1-ethyl-3-(3-dimethylaminopropyl)-carbodiimide) and NHS(N-hydrosulfosuccinimide) (Pierce Biotechnology, Rockford, Ill.). Coupling concentrations were 60 μg/ml Gag, 60 μg/ml Pol, and 15 μg/ml Nef. Rhesus sera were heat-inactivated (56° C. for 90 minutes) and diluted to 1:20 and 1:200 concentrations in phosphate buffered saline containing 10% normal goat serum; 50 μl/well was added to duplicate wells in a 96-well filter plate. Microspheres were diluted in phosphate buffered saline containing 10% normal goat serum to a concentration of 100 beads/μl for each antigen and 50 μl was added to each well. The plate was incubated on a shaker in the dark for 1 hour at room temperature and then was washed three times with wash buffer. The beads were resuspended in 100 μl of 5 μg/ml phycoerythrin-conjugated anti-human IgG monoclonal antibody and incubated on a plate shaker in the dark for 1 hour at room temperature. The plate was washed three times with wash buffer, and the beads were re-suspended in 100 μl of wash buffer, mixed on a plate shaker for five minutes and then read on a BIOPLEX™ instrument (Bio-Rad, Hercules, Calif.) according to manufacturer instructions. Median fluorescence intensities from a minimum of 100 beads were collected. A 12-point standard curve was run on the same plate composed of a dilution series of a mixture of high-titer rhesus monkeys. Samples titers were determined from a back-calculation of the standard curve fit to a 4-point logistic (sigmoidal) function. Results were expressed as units/ml and are detailed in FIGS. 21A-C.

FIGS. 21A-C illustrate the antibody levels in units/ml for Gag (a), Pol (b), and Nef (c) antigens, respectively, as a function of time of sampling in weeks post-injection. The geometric mean of each group in the vaccination protocol detailed in Example 11 is plotted. Units of Gag, Pol, and Nef are referenced to the standard curve, and so meaningful quantitative comparisons cannot be made between Gag, Pol, and Nef Units of antibody concentration.

In all cases, significant levels of antibodies to the relevant antigens are elicited, peaking in either week 4 or week 8 post-injection. In panel (a), all groups have robust anti-Gag antibody levels as expected because all groups received gag antigen vaccines. In panel (b), the groups receiving pol fusions demonstrate robust anti-Pol antibody levels; the trivalent Ad6gag+Ad6nef+Ad6pol (circle symbols) group demonstrates lower levels perhaps due to immunodominance towards Gag and Nef. The peak level is distinguishable from the groups that did not receive pol-containing vaccines. In panel (c), all groups have robust anti-Nef antibody levels except for the Ad6gagpol group (light gray square symbols with dash marks) that did not receive a nef-containing vaccine, and the MRKAd6gagpolnef fusion (diamond symbols). It is possible that the high sequence of diversity of the Nef antigen causes a lower signal in this assay due to the heterologous sequences used in the vaccine and the assay antigen. The multiple nef sequences used to vaccinate the other groups may mitigate this effect by increasing the epitope overlap with the assay antigen.

1. A method for generating consensus sequences of use in vaccination, which comprises:
(a) compiling a population of two or more sequences from a particular natural antigen sequence;
(b) deriving substantially all possible overlapping successive sequence fragments (“N-mers”) for the sequences in the population; said N-mers characterized as being of a length (“N”) which comprises at least one epitope of interest; wherein “N” is any number from about 7 to about 30; and
(c) adding successive amino acids, first to an initial N-mer (a stretch of N amino acids that begin a sequence in (a)) by identifying a fragment(s) overlapping the preceding N-mer by N−1 amino acids and adding the last amino acid of the fragment(s), repeating this procedure until ending with the final amino acid of a terminal N-mer (a stretch of N amino acids that end a sequence in (a));
wherein resultant consensus sequences have at least 90% of every successive N-mer sequence present in a natural antigen sequence.

2. A method for generating and comparing consensus sequences of use in vaccination, which comprises:
(a) compiling a population of two or more sequences from a particular natural antigen sequence;
(b) deriving substantially all possible overlapping successive sequence fragments (“N-mers”) for the sequences in the population; said N-mers characterized as being of a length (“N”) which comprises at least one epitope of interest; wherein “N” is any number from about 7 to about 30;
(c) individually assigning each fragment a weight proportional to the number of natural antigen sequences provided per patient or subject (“input sequences”);
(d) optionally, adjusting the weights of (c) according to the prevalence of each sequence within a particular clade, subtype or geographic region or according to the pathogenicity or oncogenicity of each sequence;
(e) providing a score to each fragment based on the number of times said fragment appears in the input sequences and the weight of (c) and/or (d);
(f) adding successive amino acids, first to an initial N-mer (a stretch of N amino acids that begin a sequence in (a)) by identifying a fragment(s) overlapping the preceding N-mer by N−1 amino acids and adding the last amino acid of the fragment(s), repeating this procedure until ending with the final amino acid of a terminal N-mer (a stretch of N amino acids that end a sequence in (a));
(g) calculating the cumulative total score of the successive sequence fragments of the sequences produced in step (f); and
(h) comparing the consensus sequences based on total score;
wherein resultant consensus sequences have at least 90% of every successive N-mer sequence present in a natural antigen sequence.

3. The method of claim 1 wherein the resultant sequences have at least 95% of every successive N-mer sequence present in a natural antigen sequence.

4-6. (canceled)

7. The method of claim 1 wherein the consensus sequences are viral consensus sequences.

8. The method of claim 7 wherein the viral consensus sequences are derived from an Human Immunodeficiency Virus (“HIV”) antigen.

9-10. (canceled)

11. The method of claim 1 wherein the N-mer is selected from the group consisting of: (1) an 8-mer, (2) a 9-mer, (3) a 15-mer and (4) a 16-mer.

12. (canceled)

13. The method of claim 1 wherein the N-mer is a 16-mer.

14. (canceled)

15. A consensus antigen sequence wherein at least 90% of every possible successive sequence of “N” amino acids (“N-mer”) therein is present in a natural antigen sequence; wherein “N” is any number from about 7 to about 30; wherein the consensus antigen sequence comprises N-mer sequence from at least three different natural antigen sequences; and wherein the consensus antigen sequence is not found in a natural antigen sequence.

16. The consensus antigen sequence of claim 15 wherein at least 95% of every successive N-mer sequence therein is present in a natural antigen sequence.

17. (canceled)

18. The consensus antigen sequence of claim 15 wherein the N-mer is selected from the group consisting of: (1) an 8-mer, (2) a 9-mer, (3) a 15-mer, (4) a 16-mer, and (5) a 30-mer.

19. The consensus antigen sequence of claim 15 wherein the antigen sequence is a viral antigen sequence.

20-24. (canceled)

25. Isolated nucleic acid encoding the consensus antigen sequence of claim 15.

26. (canceled)

27. A vector comprising the isolated nucleic acid of claim 25.

28. (canceled)

29. A cell or population of cells comprising the isolated nucleic acid of claim 25.

30. (canceled)

31. A method for inducing a cell-mediated immune response against an antigen which comprises delivery and expression of isolated nucleic acid encoding the consensus antigen sequence of claim 15.

32. (canceled)

33. A recombinant polypeptide comprising the consensus antigen sequence of claim 15.

34. (canceled)

35. A method for inducing a cell-mediated immune response against an antigen which comprises delivery and expression of the recombinant polypeptide of claim 33.

36. (canceled)

37. The method of claim 31 wherein the antigen is HIV-1 Gag.

38-39. (canceled)

40. The method of claim 31 wherein delivery and expression is of two or more sequences; said two or more sequences encoding two or more antigens from a set of sequences selected from the group consisting of: (1) SEQ ID NO: 64, SEQ ID NO: 65 and SEQ ID NO: 66; (2) SEQ ID NO: 46, SEQ ID NO: 67 and SEQ ID NO: 68; (3) SEQ ID NO: 69, SEQ ID NO: 70 and SEQ ID NO: 71; (4) SEQ ID NO: 70, SEQ ID NO: 1 and SEQ ID NO: 2; (5) SEQ ID NO: 72, SEQ ID NO: 73 and SEQ ID NO: 74; (6) SEQ ID NO: 70; SEQ ID NO: 75 and SEQ ID NO: 76; (7) SEQ ID NO: 77, SEQ ID NO: 78 and SEQ ID NO: 79; (8) SEQ ID NO: 80, SEQ ID NO: 81 and SEQ ID NO: 82; (9) SEQ ID NO: 83, SEQ ID NO: 84 and SEQ ID NO: 85; (10) SEQ ID NO: 80, SEQ ID NO: 3 and SEQ ID NO: 4; (11) SEQ ID NO: 86, SEQ ID NO: 87 and SEQ ID NO: 88; (12) SEQ ID NO: 80, SEQ ID NO: 89 and SEQ ID NO: 90.

41-42. (canceled)

43. Isolated nucleic acid encoding at least one Human Immunodeficiency Virus (“HIV”) antigen; said antigen comprising an amino acid sequence selected from the group consisting of: SEQ ID NO: 1, SEQ ID NO: 2, SEQ ID NO: 3, SEQ ID NO: 4, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 61, SEQ ED NO: 62, SEQ ID NO: 63, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110 and fusions comprising two or more of the foregoing sequences.

44. The isolated nucleic acid of claim 43 which comprises a string of nucleotides encoding a sequence selected from the group consisting of: SEQ ID NO: 1 and SEQ ID NO: 2.

45. The isolated nucleic acid of claim 43 which comprises a sequence selected from the group consisting of: SEQ ID NO: 39, SEQ ID NO: 40, SEQ ID NO: 41, SEQ ID NO: 42, SEQ ID NO: 43, SEQ ID NO: 44 and SEQ ID NO: 45.

46-50. (canceled)

51. The isolated nucleic acid of claim 43 which further comprises at least one nucleic acid encoding an amino acid sequence selected from the group consisting of: SEQ ID NO: 46, SEQ ID NO: 80, SEQ ID NO: 100 and SEQ ID NO: 112.

52. The isolated nucleic acid of claim 43 which further comprises at least one nucleic acid selected from the group consisting of: SEQ ID NO: 47, SEQ ID NO: 113 and SEQ ID NO: 111.

53. (canceled)

54. A vector which comprises the isolated nucleic acid of claim 43.

55-61. (canceled)

62. A method for inducing a cell-mediated immune response against an HIV antigen which comprises delivery and expression of the isolated nucleic acid of claim 43.

63. The method of claim 62 which comprises the delivery and expression of a vector comprising the isolated nucleic acid of claim 43.

64-68. (canceled)

69. A cell or population of cells transfected with the isolated nucleic acid of claim 43.

70-71. (canceled)

72. A recombinant polypeptide which comprises at least one amino acid sequence selected from the group consisting of: SEQ ID NO: 1, SEQ ID NO: 2, SEQ ID NO: 3, SEQ ID NO: 4. SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 61, SEQ ID NO: 62, SEQ ID NO: 63, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110 and fusions of two or more of the foregoing sequences.

73. (canceled)

74. The recombinant polypeptide of claim 72 which further comprises at least one amino acid sequence selected from the group consisting of: SEQ ID NO: 46, SEQ ID NO: 80, SEQ ID NO: 100 and SEQ ID NO: 112.

75. (canceled)

76. A method for inducing a cell-mediated immune response against an HIV antigen which comprises administration of the recombinant polypeptide of claim 72.

77. Recombinant, replication-defective adenovirus comprising two or more isolated nucleic acid sequences; said two or more sequences encoding two or more antigens from a set of sequences selected from the group consisting of: (1) SEQ ID NO: 64, SEQ ID NO: 65 and SEQ ID NO: 66; (2) SEQ ID NO: 46, SEQ ID NO: 67 and SEQ ID NO: 68; (3) SEQ ID NO: 69, SEQ ID NO: 70 and SEQ ID NO: 71; (4) SEQ ID NO: 70, SEQ ID NO: 1 and SEQ ID NO: 2; (5) SEQ ID NO: 72, SEQ ID NO: 73 and SEQ ID NO: 74; (6) SEQ ID NO: 70; SEQ ID NO: 75 and SEQ ID NO: 76; (7) SEQ ID NO: 77, SEQ ID NO: 78 and SEQ ID NO: 79; (8) SEQ ID NO: 80, SEQ ID NO: 81 and SEQ ID NO: 82; (9) SEQ ID NO: 83, SEQ ID NO: 84 and SEQ ID NO: 85; (10) SEQ ID NO: 80, SEQ ID NO: 3 and SEQ ID NO: 4; (11) SEQ ID NO: 86, SEQ ID NO: 87 and SEQ ID NO: 88; (12) SEQ ID NO: 80, SEQ ID NO: 89 and SEQ ID NO: 90.
